(19) United States
(12) Patent Application Publication
(10) Pub. No.: US 2014/0228293 A1
(43) Pub. Date: Aug. 14, 2014

(54) PARATHYROID HORMONE ANALOGS, COMPOSITIONS AND USES THEREOF

(71) Applicants: Samuel J. Danishefsky, Englewood, NJ (US); Shiying Shang, Superior, CO (US); Zhongping Tan, Superior, CO (US); Suwei Dong, New York, NY (US); Jianfeng Li, New York, NY (US); Thomas Gardella, Needham, MA (US)

(72) Inventors: Samuel J. Danishefsky, Englewood, NJ (US); Shiying Shang, Superior, CO (US); Zhongping Tan, Superior, CO (US); Suwei Dong, New York, NY (US); Jianfeng Li, New York, NY (US); Thomas Gardella, Needham, MA (US)

(73) Assignees: THE GENERAL HOSPITAL CORPORATION, Boston, MA (US); SLOAN-KETTERING INSTITUTE FOR CANCER RESEARCH, New York, NY (US)

(21) Appl. No.: 14/002,601

(22) PCT Filed: Mar. 1, 2012
(86) PCT No.: PCT/US12/27339

§ 371 Date: Sep. 27, 2013

Related U.S. Application Data
(60) Provisional application No. 61/448,064, filed on Mar. 1, 2011.

(51) Int. Cl. C07K 14/635; C07K 7/08; C07K 14/00

(52) U.S. Cl. CPC C07K 14/635; C07K 14/001; C07K 7/08
USPC 514/11.8; 530/324; 530/326; 530/322; 530/327

(57) ABSTRACT

The present invention provides parathyroid hormone and/or parathyroid hormone-related protein analogs, compositions thereof and methods thereto.

### CROSS REFERENCE TO RELATED APPLICATIONS

The present application claims priority to U.S. Provisional Application No. 61/448,064, filed Mar. 1, 2011, which is hereby incorporated by reference in its entirety.

### GOVERNMENT SUPPORT STATEMENT

The present invention was supported in part by Grant No. Ca28824-33 from the National Institutes of Health and NIDDK-11794 The United States Government has certain rights in this invention.

### SEQUENCE LISTING

In accordance with PCT Rule 5.2, a Sequence Listing in the form of a text file (entitled “Sequence_Listing_ST25.txt,” created on Feb. 28, 2012, and 17 kilobytes in size) is submitted herewith and incorporated herein by reference in its entirety.

### BACKGROUND OF THE INVENTION

Human Parathyroid Hormone (hPTH) is a biological messenger that is secreted by the parathyroid gland as a peptide containing 84-amino acids. hPTH is the most important endocrine regulator of calcium and phosphorous concentration in extracellular fluid. If calcium ion concentrations (Ca²) in extracellular fluid fall below normal, hPTH can restore the levels to within normal range by stimulating bone resorption, enhancing reabsorption of calcium in the kidneys and intestines and/or suppressing calcium loss in urine. In conjunction with increasing calcium concentration, the concentration of phosphate ion in the blood is reduced. Low levels of hPTH are secreted even when blood calcium levels are high.

Decreased function of the parathyroid gland leads to hypoparathyroidism and decreased levels of parathyroid hormone. The resulting hypocalcemia produces such symptoms as tingling of fingers and toes, muscle cramps and spasms, convulsions, pain and dry skin. Although hypoparathyroidism results in increased bone density, it is also associated with a higher frailty status believed to result from faulty bone remodeling in the absence of parathyroid hormone activity. Further, while chronic secretion or continuous infusion of parathyroid hormone leads to bone decalcification, and to loss of bone mass, in certain situations, treatment with recombinant parathyroid hormone can actually stimulate an increase in bone mass and bone strength. This seemingly paradoxical effect occurs when the hormone is administered in pulses (e.g. by once daily injection), and such treatment appears to be an effective therapy for diseases such as osteoporosis.

### SUMMARY OF THE INVENTION

The present invention provides new hPTH peptides and/or analogs with desirable characteristics. In some embodiments, provided hPTH peptides and/or analogs include one or more non-natural amino acid residues. In certain embodiments, provided hPTH peptides and/or analogs include one or more norleucine and/or methoxinine residues. In some embodiments, provided hPTH peptides and/or analogs include one or more norleucine and/or methoxinine residues in a substantially full-length hPTH. In some embodiments, provided hPTH peptides and/or analogs include one or more norleucine and/or methoxinine residues at positions corresponding to residue 8 and/or residue 18 of SEQ ID NO: 2.

In some embodiments, provided hPTH peptides and/or analogs have at least 80% overall sequence identity with SEQ ID NO: 1 or SEQ ID NO: 2.

In some embodiments, provided hPTH peptides and/or analogs are glycosylated. In some embodiments, provided hPTH peptides and/or analogs are O-glycosylated. In some embodiments, provided hPTH peptides and/or analogs are N-glycosylated. In some embodiments, provided hPTH peptides and/or analogs are glycosylated at positions corresponding to residue 1 and/or residue 33 of SEQ ID NO: 1 or SEQ ID NO: 2. In some embodiments, provided hPTH peptides and/or analogs are glycosylated with one or more glycans selected from the group consisting of carbohydrates that are commonly used in the chemical synthesis of glycoproteins.

Among other things, the present invention encompasses the recognition that increasing the stability and half-life of hPTH therapies facilitates more tolerable administration and greater patient compliance. In some embodiments, the present invention provides more stable hPTH therapeutics. In some embodiments, provided hPTH analogs have greater stability than hPTH of SEQ ID NO: 1 (e.g., when measured in an in vitro peptide stability assay in human serum).

In some embodiments, the present invention also provides pharmaceutical compositions comprising one or more provided hPTH peptides and/or analogs and at least one pharmaceutically acceptable excipient.

In certain embodiments, provided hPTH peptides and/or analogs and/or compositions containing them are useful in medicine, for example in methods of treating a disease, disorder, or condition associated with insufficient levels of parathyroid hormone. Among other things, the present invention provides methods of treatment comprising administering a provided composition or hPTH peptides and/or analogs to a subject in need thereof.

The present invention also encompasses native chemical ligation technologies that do not rely on cysteine and/or methionine residues. In some embodiments, the present invention provides native chemical ligation technologies for the production of peptides or peptide analogs that do not include useful cysteine and/or methionine residues. In some embodiments, the present invention provides native chemical ligation technologies for the production of one or more hormones that not do include useful cysteine and/or methionine residues. In some embodiments the present invention provides native chemical ligation technologies for the production of hPTH peptides and/or analogs.

Native chemical ligation technologies provided as described herein include, for example, methods of preparing agents by chemical ligation, reagents involved in chemical ligation reactions, and/or intermediates developed and/or utilized in chemical ligation syntheses.

### BRIEF DESCRIPTION OF THE DRAWINGS

FIG. 1 depicts a retrosynthetic analysis of hPTH (1-84).

FIG. 2 depicts a chemical synthesis of human parathyroid hormone: (a) H-Trp-SPh, EDCI, HOOBt, DIEA, DMSO, 3 h; (b) TFA:TIS:H₂O (95:2.5:2.5), 45 min; (c) Boc-Leu(SSMe)-OH, HATU, DIEA, DMSO, 1 h; (d) TFE:AcOH:CH₂Cl₂ (8:1:1), 2 h; (e) H-Gly-SCH₂CH₂CO₂Et, EDCI, HOOBt, DIEA, DMSO, 1 h; (f) H-Leu-SPh, EDCI, HOOBt, DIEA, DMSO, 2 h; (g) Boc-Val(SSMe)-OH, HATU, DIEA, DMSO, 1 h; (h) 6 M Gn.HCl, 100 mM NaH₂PO₄, and 50 mM TCEP, pH 7.5, 9 h; (i) MeONH₂.HCl, pH 4, 2.5 h; (j) 6 M Gn.HCl, 300 mM NaH₂PO₄, 200 mM MPAA, and 20 mM TCEP, pH 7.9; (k) VA-044, tBu-SH, TCEP, H₂O, MeCN, 37° C., 2 h.

FIG. 3 depicts a chemical synthesis of [Nle^{8,18}]hPTH (1-34)

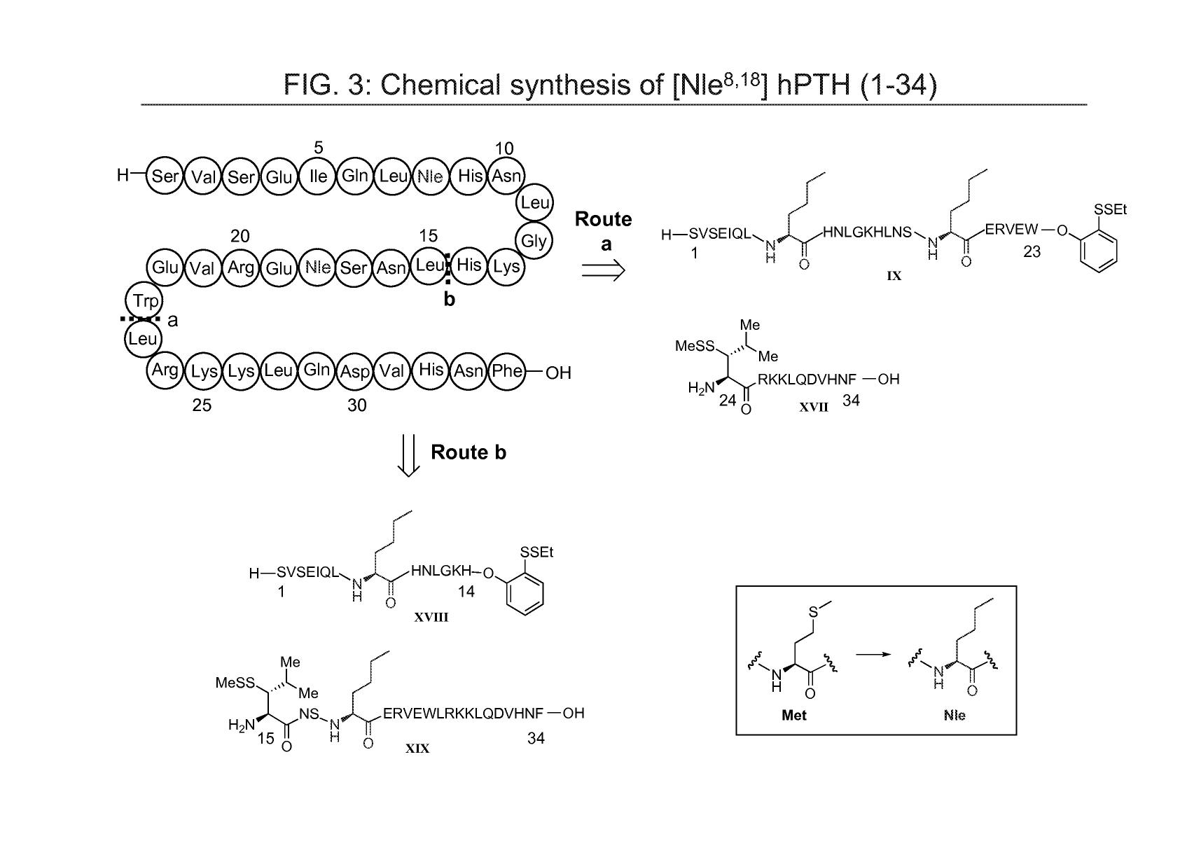

FIG. 4 depicts a chemical synthesis of O-glycosylated [Nle^{8,18}]hPTH (1-34).

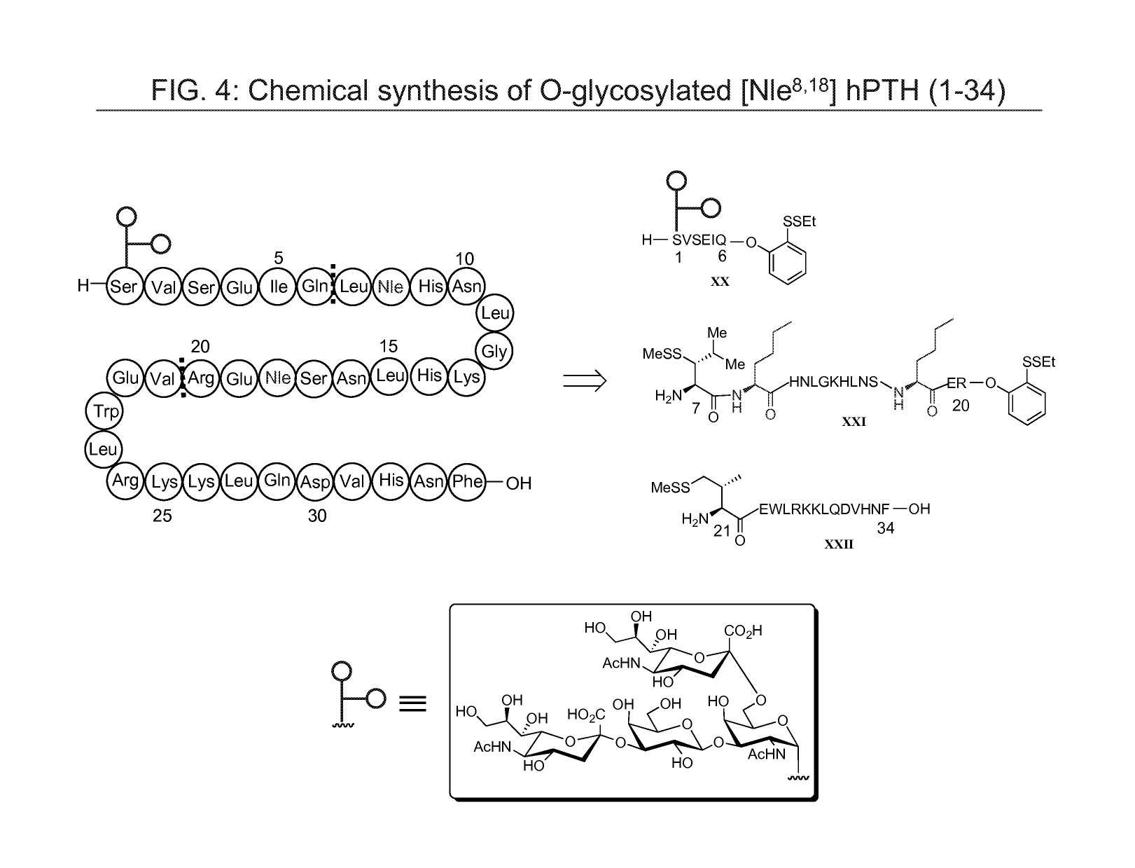

FIG. 5 depicts a chemical synthesis of N-glycosylated [Nle^{8,18}]hPTH (1-34).

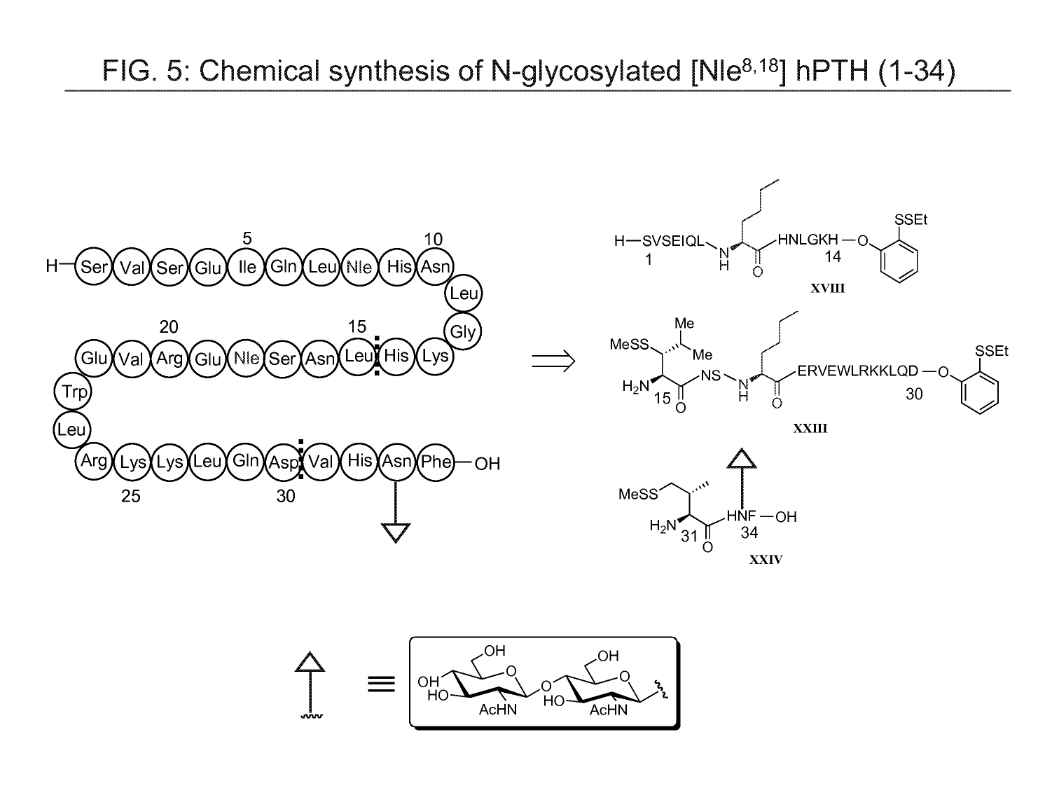

FIG. 6 depicts a chemical synthesis of N-glycosylated [Nle^{8,18}]hPTH (1-34).

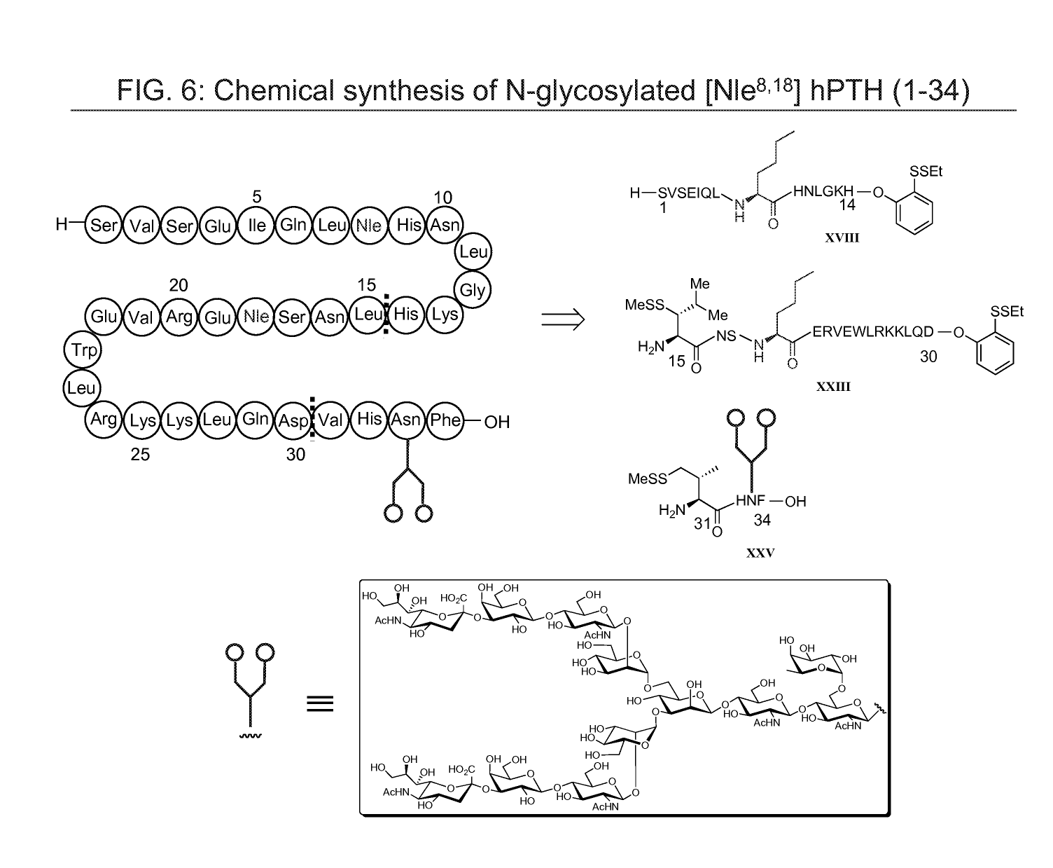

FIG. 7 depicts a chemical synthesis of [Nle^{8,18}]hPTH (1-84).

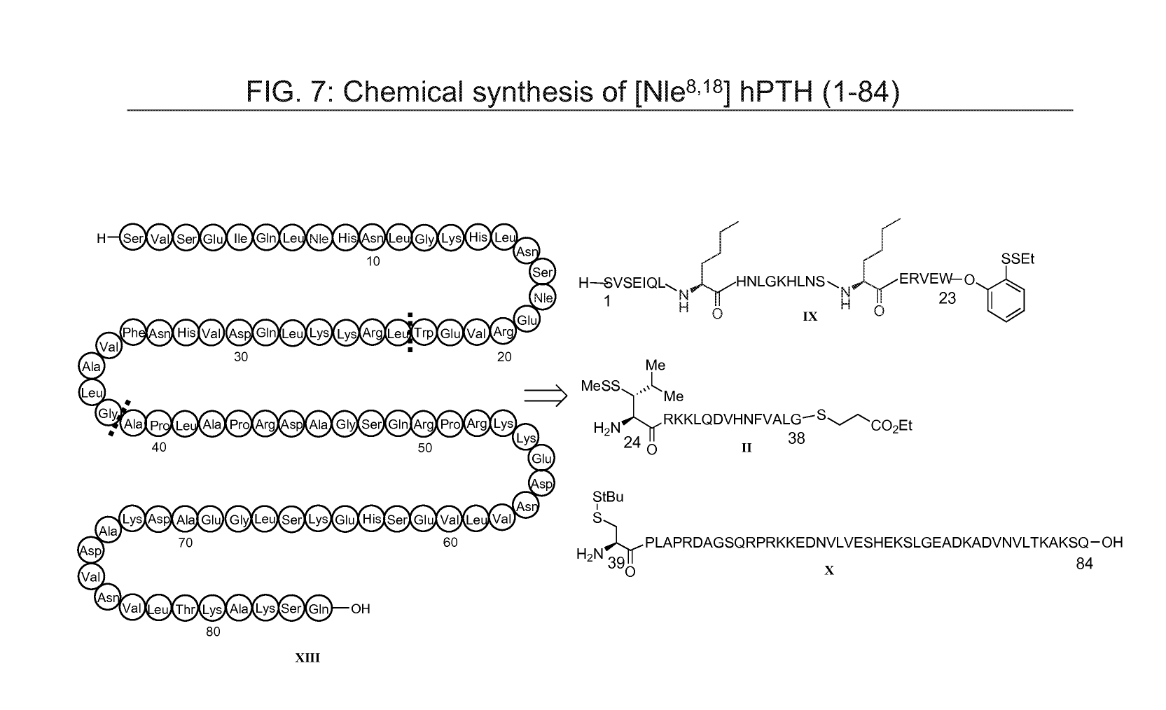

FIG. 8 depicts a chemical synthesis of O-glycosylated [Nle^{8,18}]hPTH. (1-84).

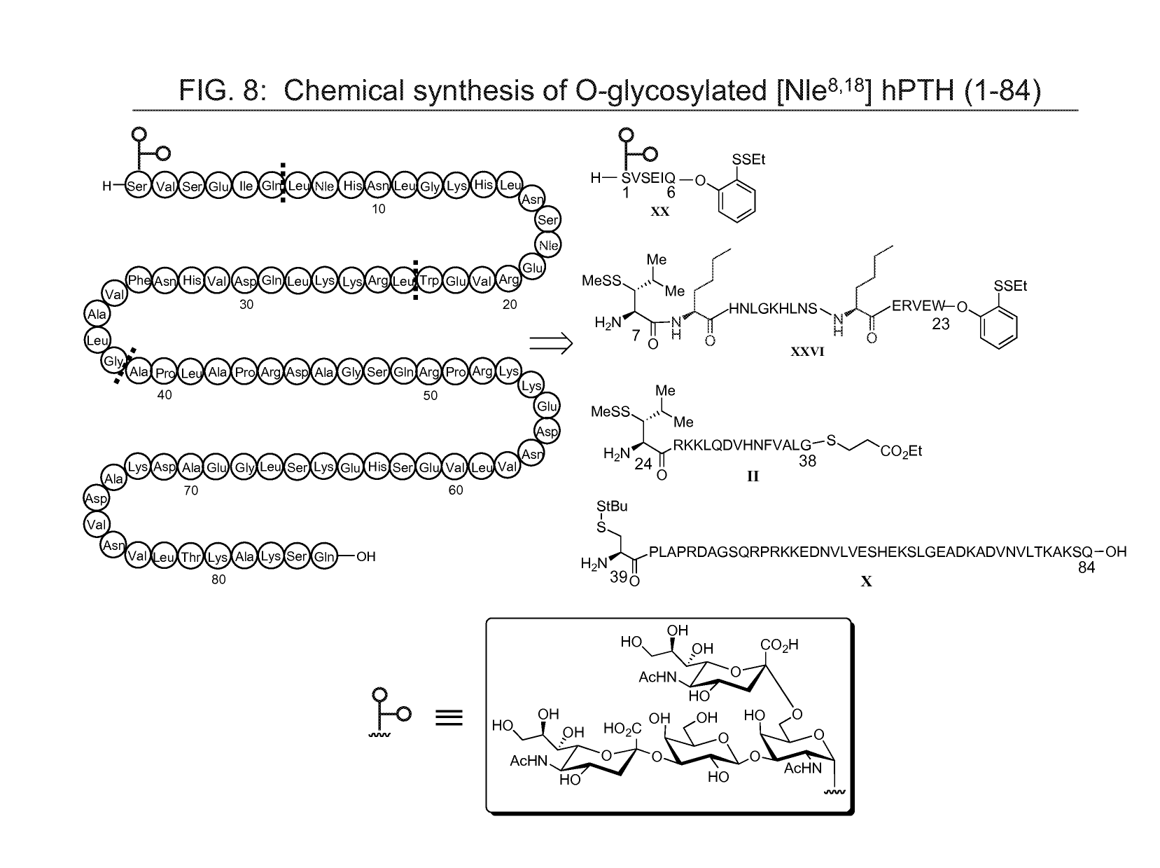

FIG. 9 depicts a chemical synthesis of N-glycosylated [Nle^{8,18}]hPTH (1-84).

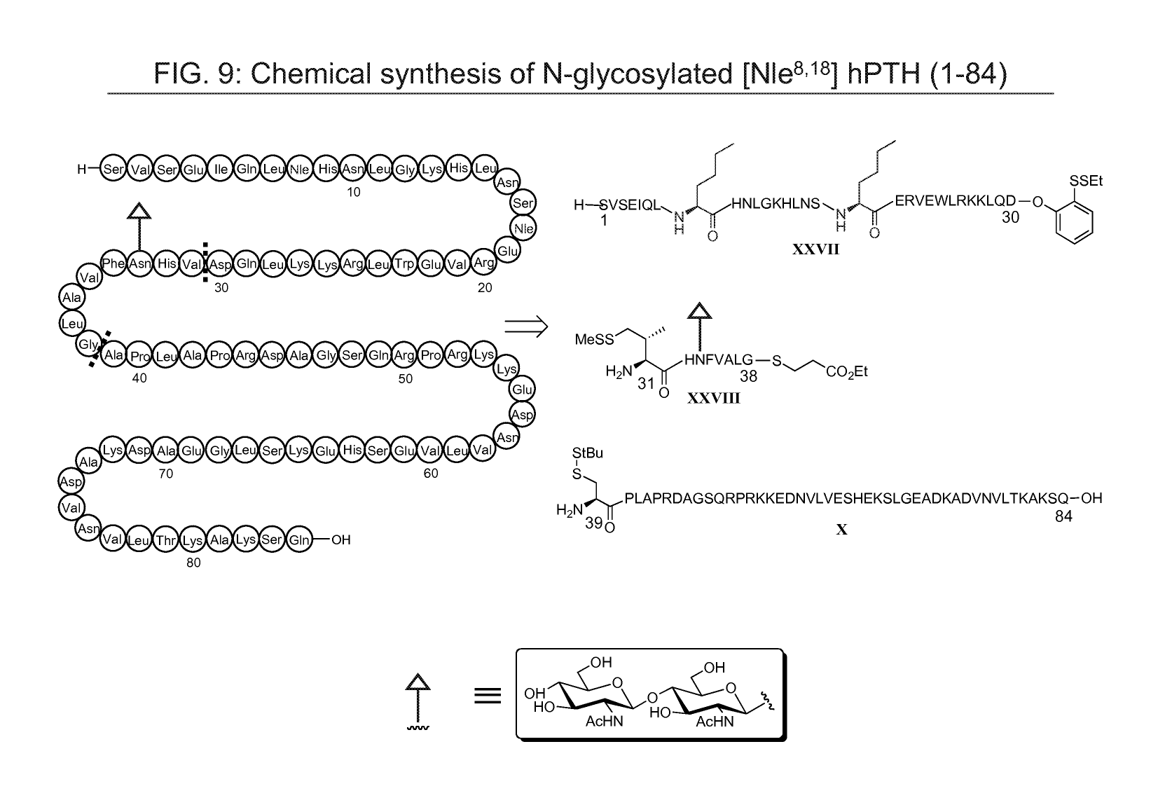

FIG. 10 depicts a chemical synthesis of N-glycosylated [Nle^{8,18}]hPTH (1-84).

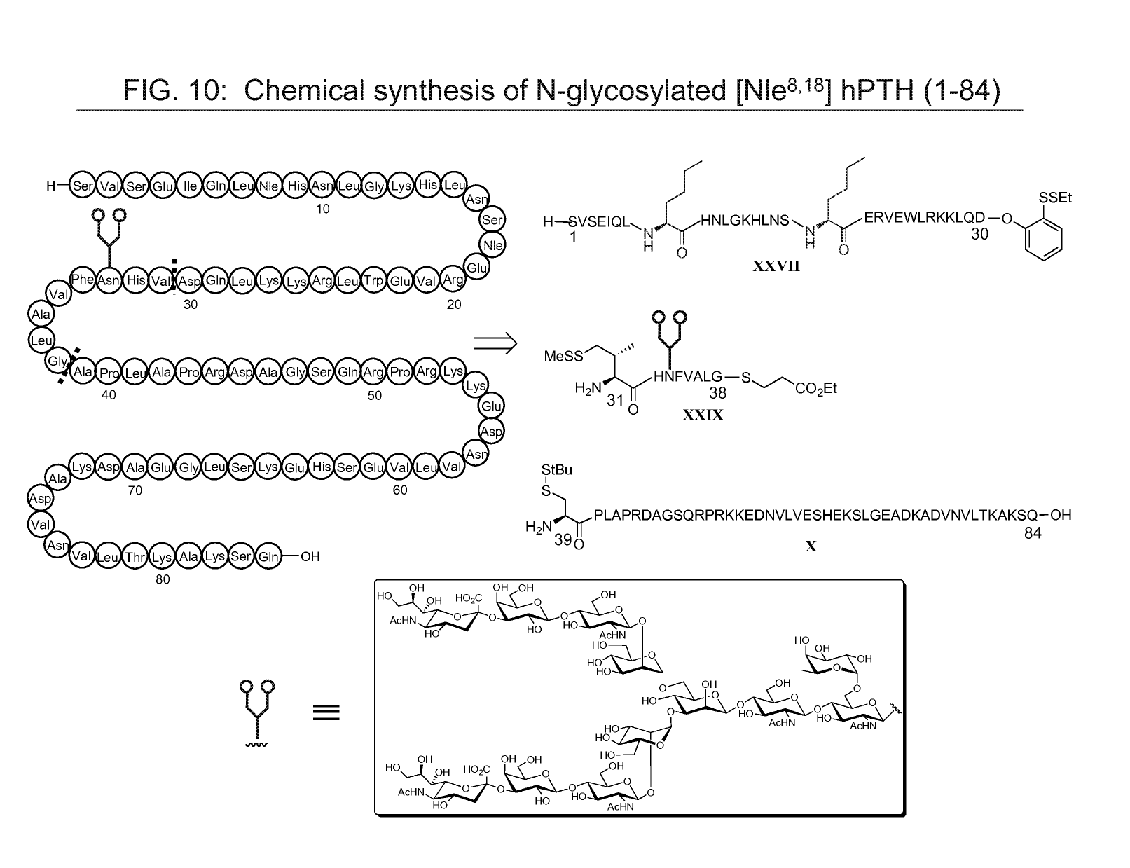

FIG. 11 depicts a retrosynthetic analysis of hPTHrP (1-141).

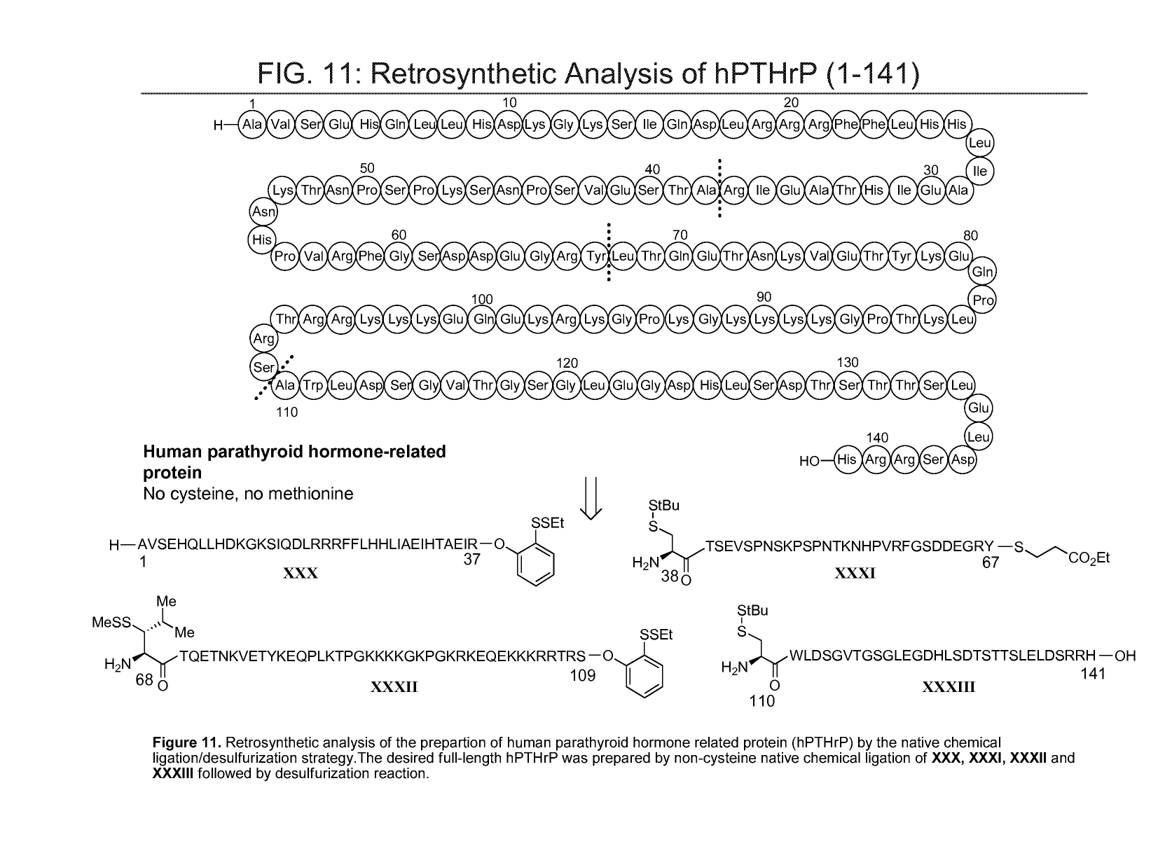

FIG. 12 depicts a chemical synthesis of hPTHrP (1-141): (a) HCl.H₂N-Arg(Pbf)-O-(2-SSEt)-Ph, HOOBt, EDC, CHCl₃, TFE, rt; (b) Cocktail B (10 mL trifluoroacetic acid [TFA], 200 mg phenol, 0.66 mL H₂O and 0.46 mL triisopropylsilane [TIS]), rt; (c) H₂N-Tyr(tBu)-S(CH₂)₂CO₂Et, HOOBt, EDC, CHCl₃, TFE, rt; (d) Boc-Leu(SSMe)-OH, HATU, DIEA, DMF, rt; (e) HOAc/TFE/DCM (1:1:8), rt; (f) HCl.H₂N-Ser(tBu)-O-(2-SSEt)-Ph, HOOBt, EDC, CHCl₃, TFE, rt; (g) TCEP, pH 7.2 buffer, rt; (h) TCEP, MPAA, pH 7.2 buffer, rt; (i) TCEP, t-BuSH, VA-044, 37° C.

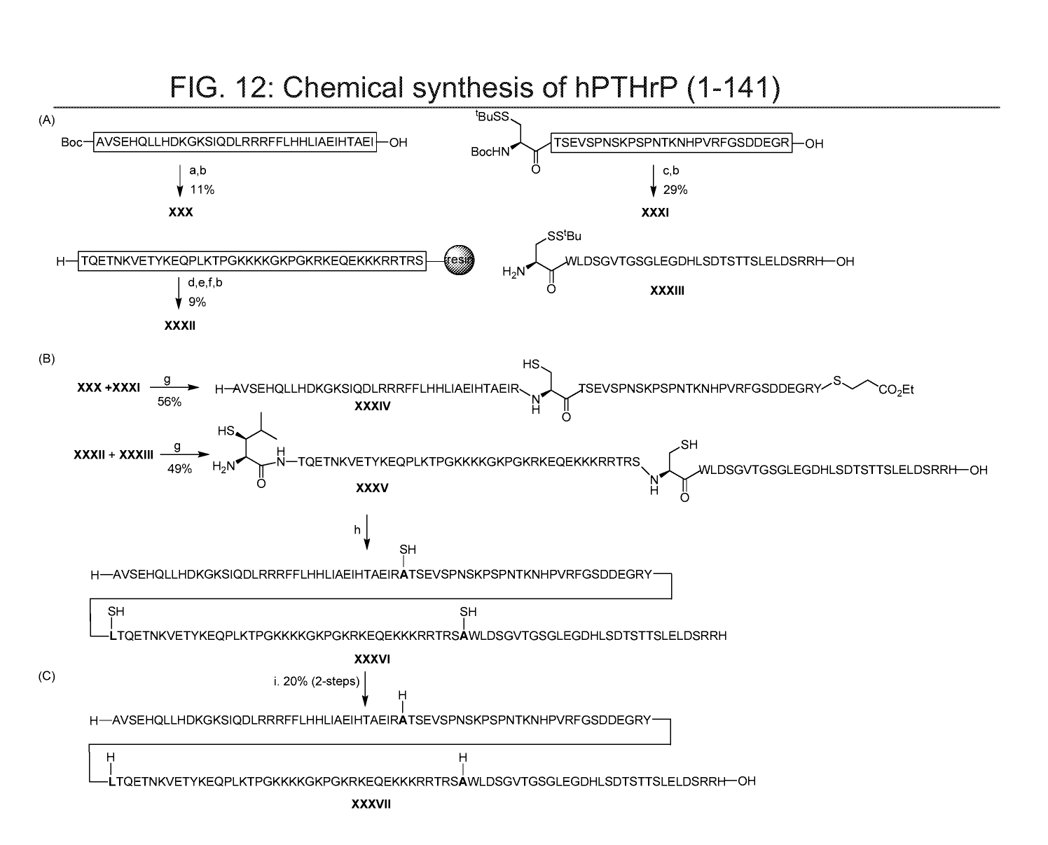

FIG. 13 presents a circular dichroism spectra of hPTH. Unnormalized Circular dichroism spectra of hPTH. Nadirs at 208 and 222 nm are characteristic of α-helical structures. Key: (a) CD comparison of the synthetic and recombinant PTH at concentration of 14 μM; (b) CD spectra of synthetic PTH at concentration of 14 μM and 7 μM.

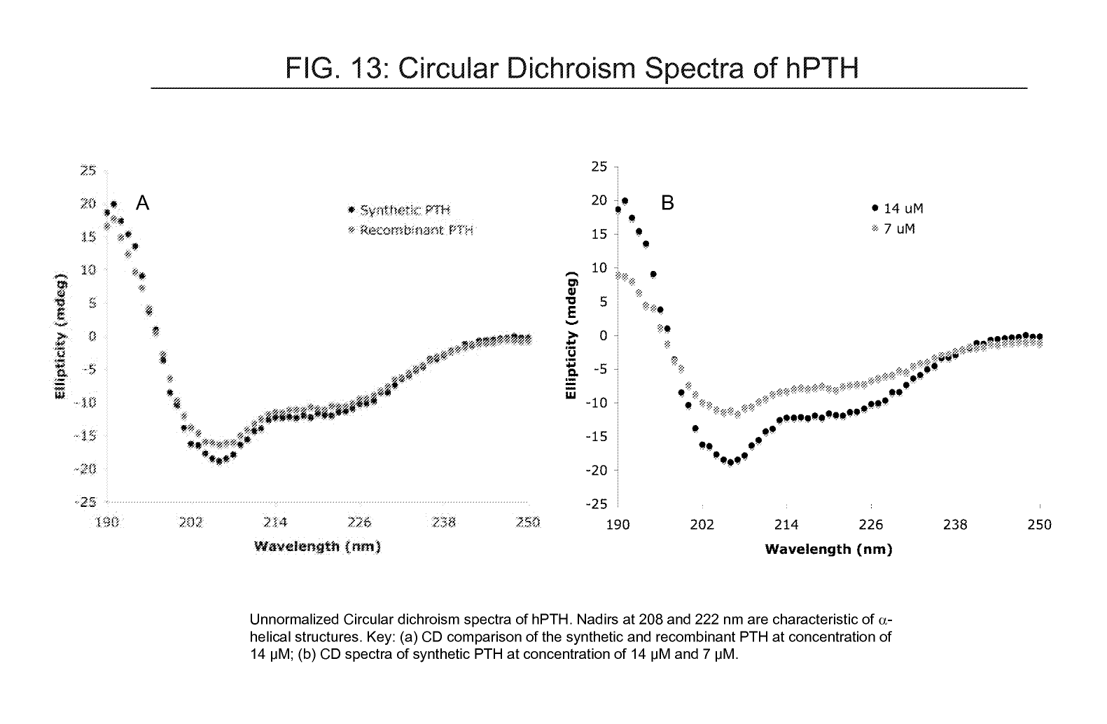

FIG. 14 presents HPLC and LC/MS spectra of hPTH (1-84) fragment I.

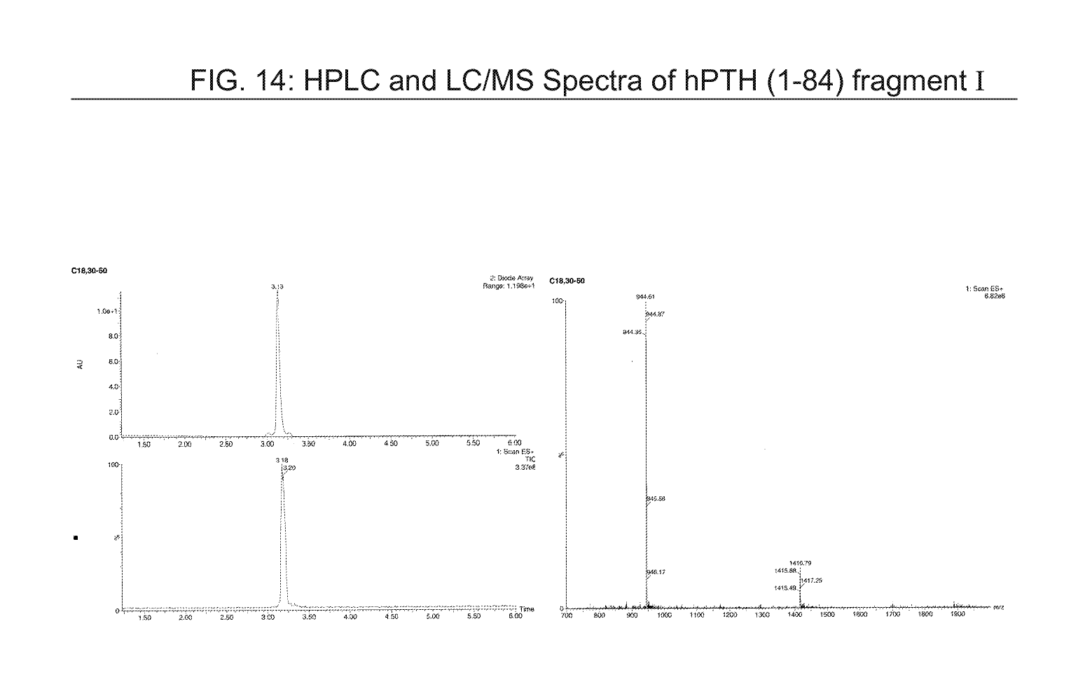

FIG. 15 presents HPLC and LC/MS spectra of hPTH (1-84) fragment II.

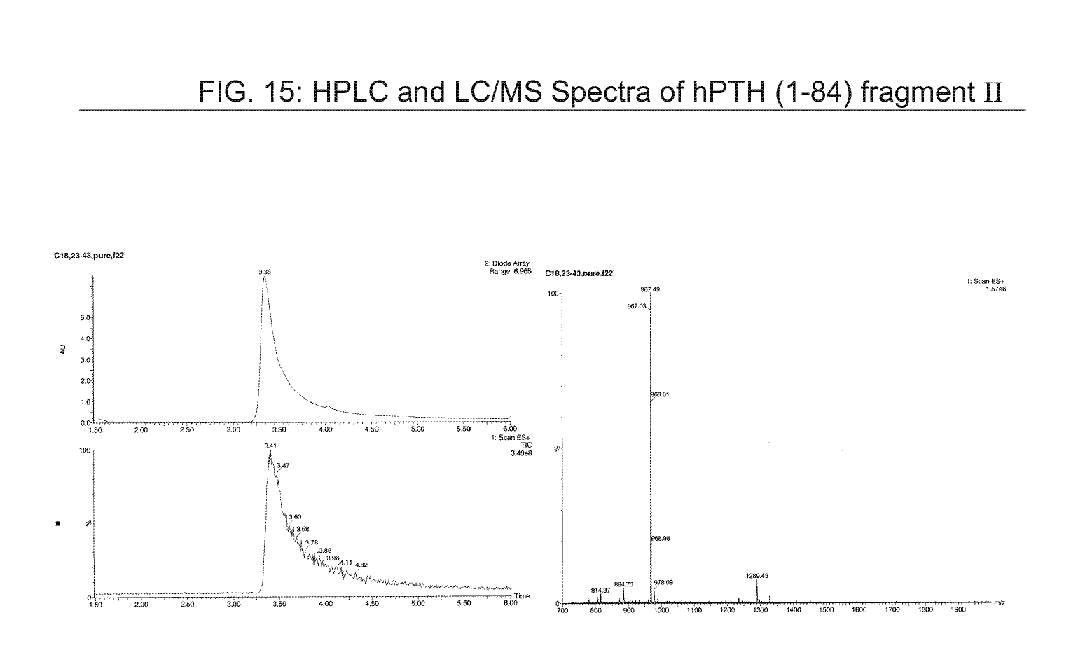

FIG. 16 presents HPLC and LC/MS spectra of hPTH (1-84) fragment III.

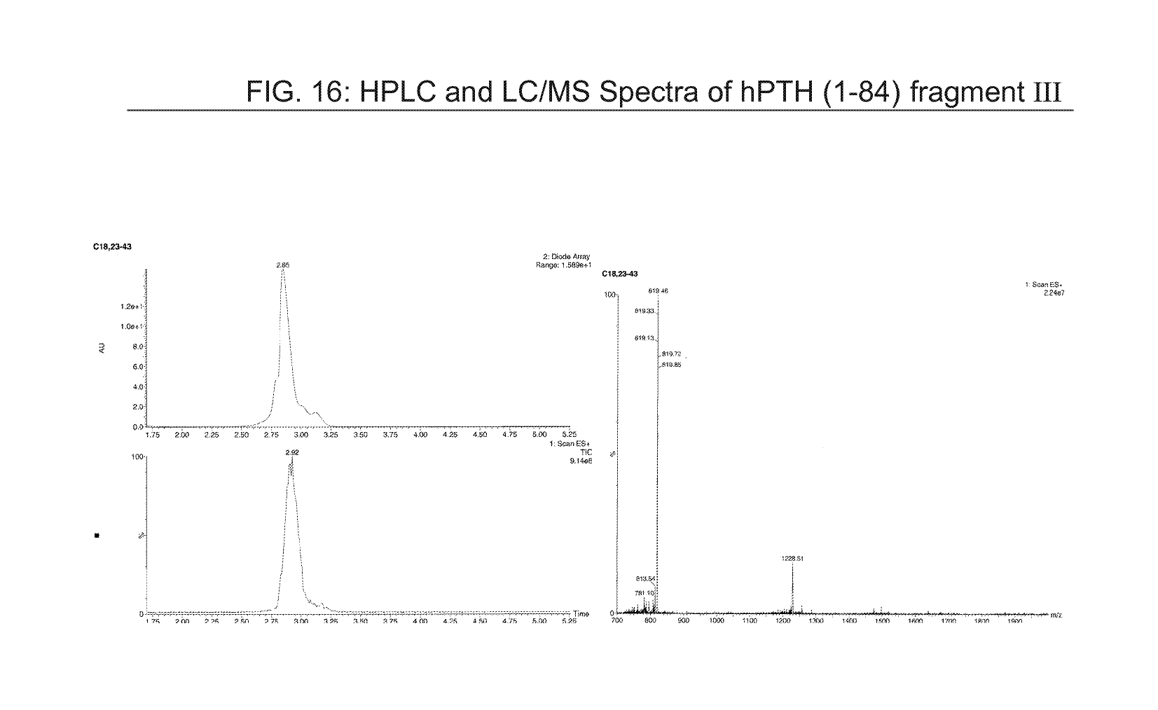

FIG. 17 presents HPLC and LC/MS spectra of hPTH (1-84) fragment IV.

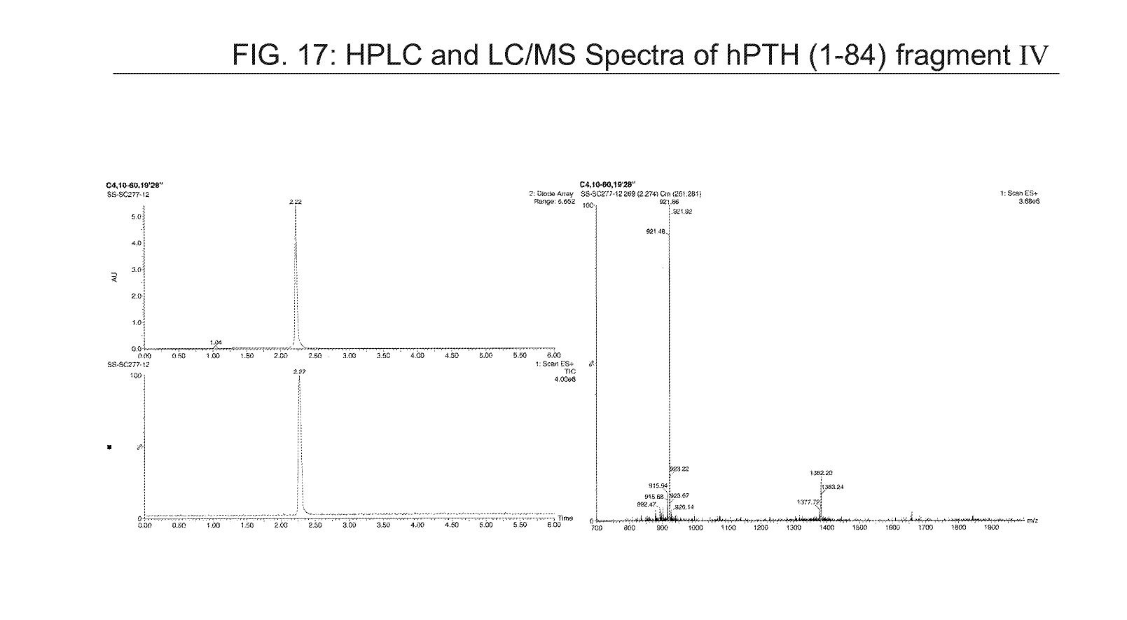

FIG. 18 presents HPLC and LC/MS spectra of hPTH (1-84) fragment V.

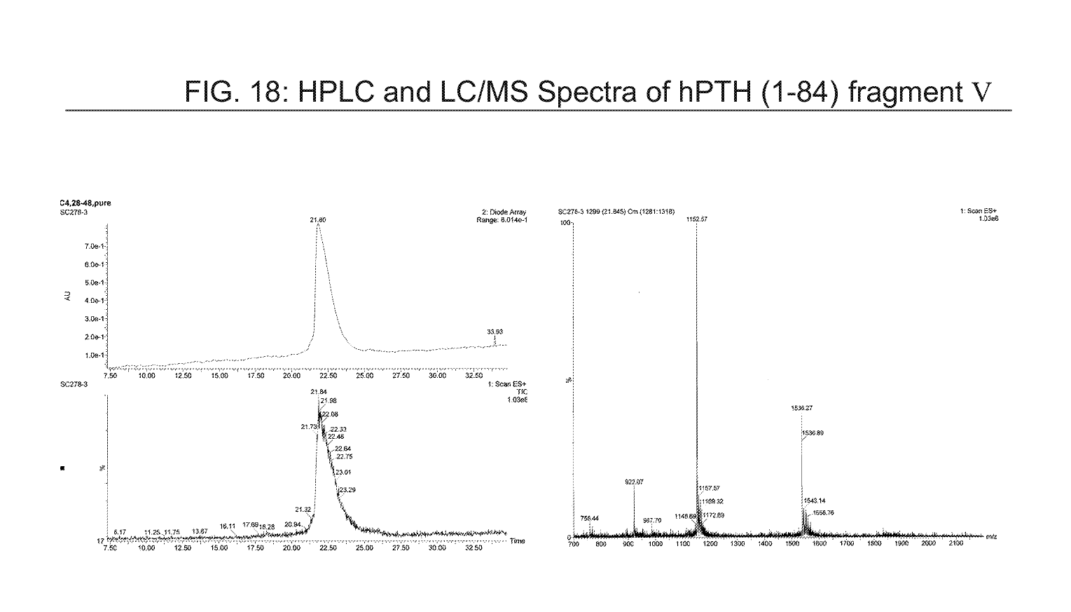

FIG. 19 presents HPLC and LC/MS spectra of hPTH (1-84) fragment VII.

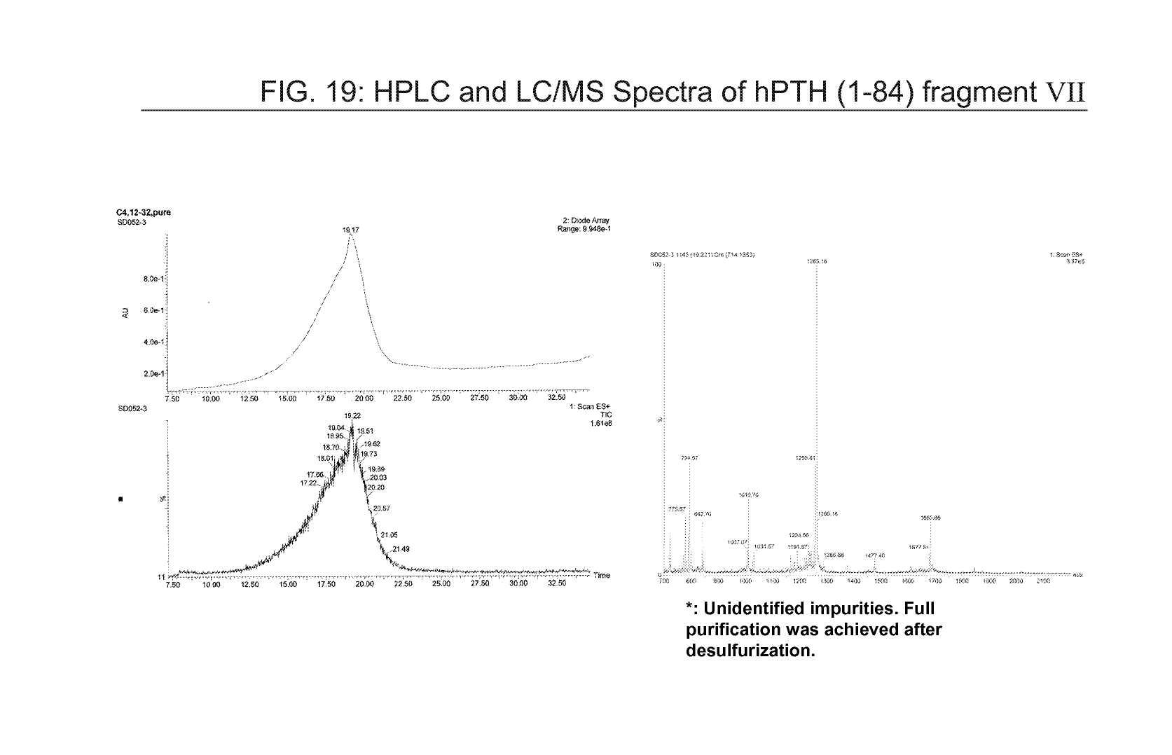

FIG. 20 presents HPLC and LC/MS spectra of hPTH (1-84) fragment VIII.

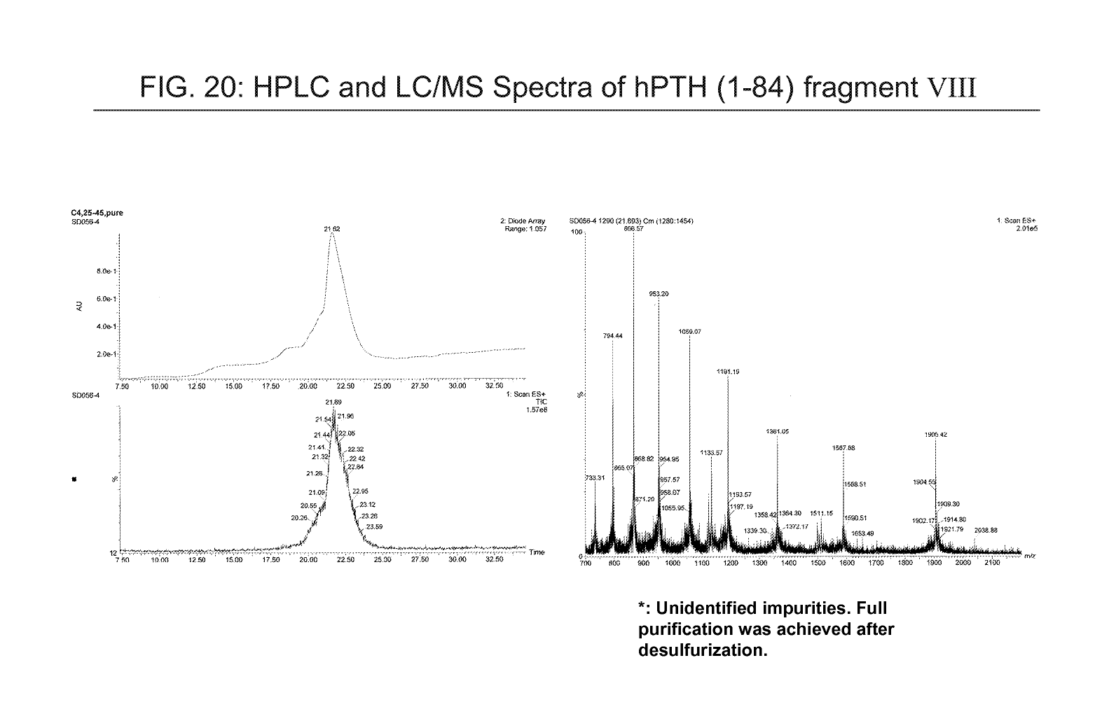

FIG. 21 presents HPLC and LC/MS spectra of hPTH (1-84).

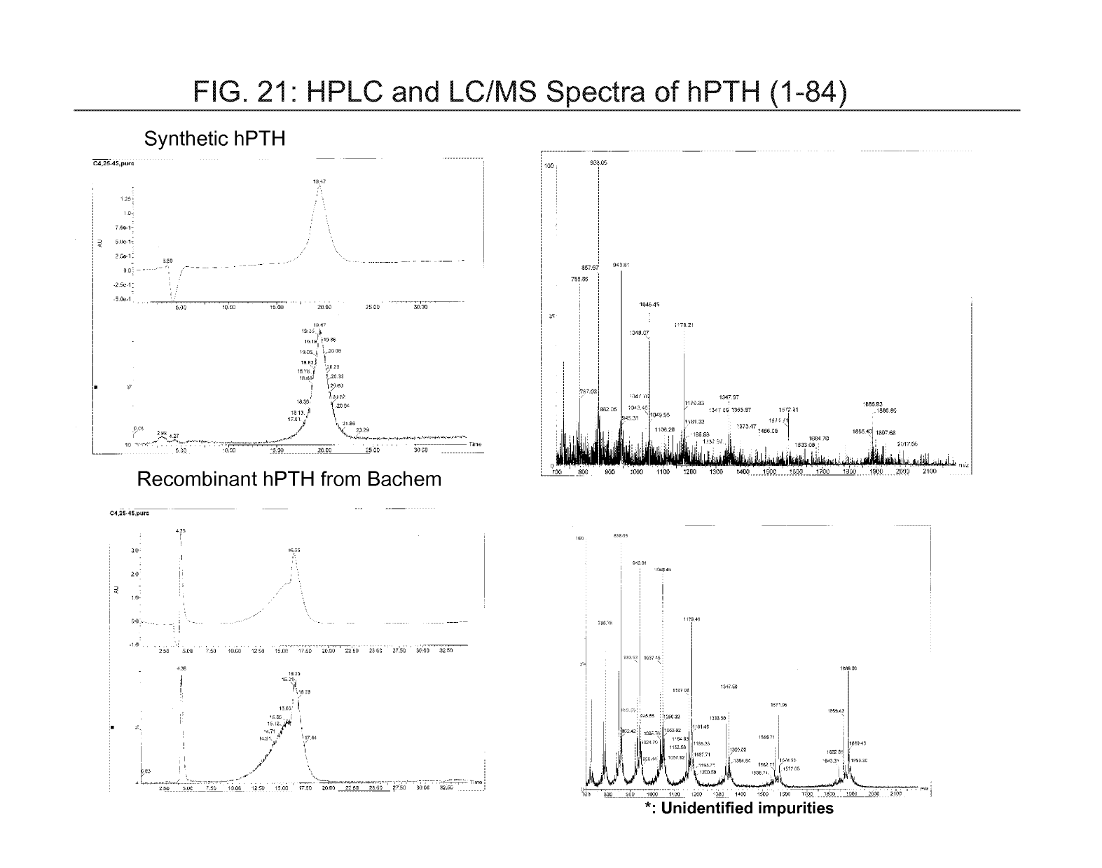

FIG. 22 presents HPLC and LC/MS spectra of [Nle^{8,18}]hPTH (1-84) fragment IX.

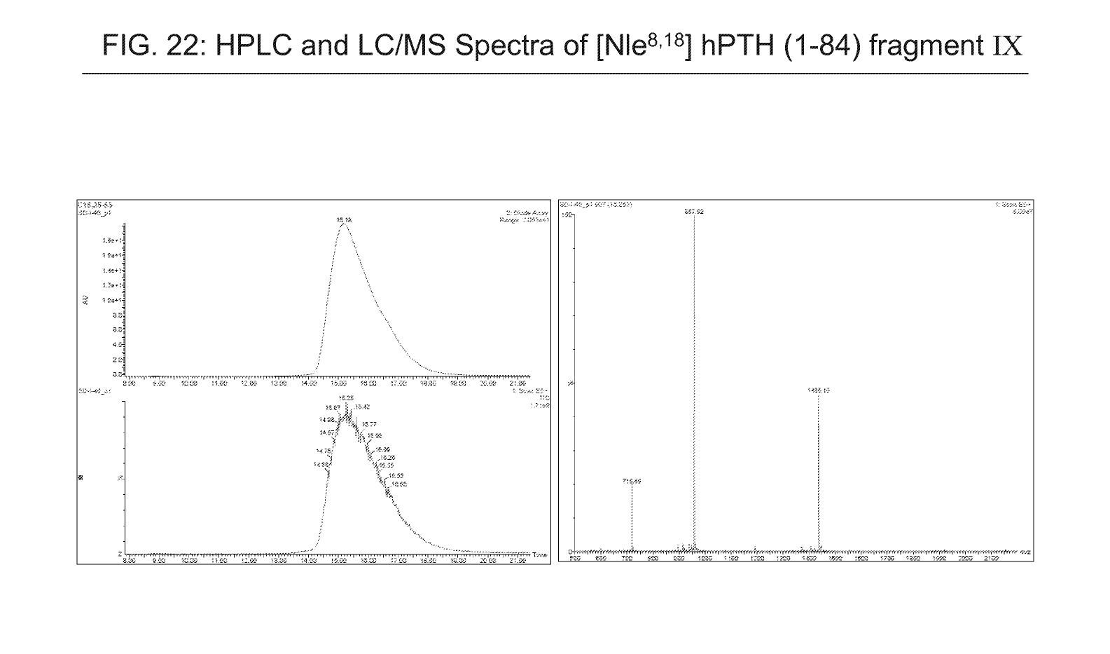

FIG. 23 presents HPLC and LC/MS spectra of [Nle^{8,18}]hPTH (1-84) fragment X.

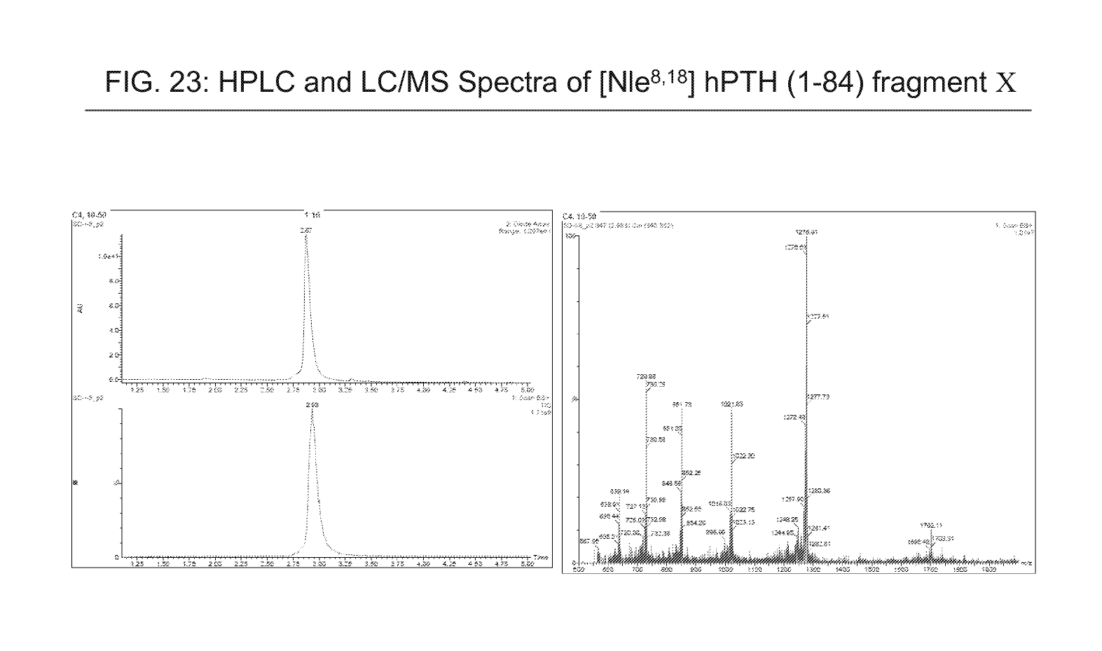

FIG. 24 presents HPLC and LC/MS spectra of [Nle^{8,18}]hPTH (1-84) fragment XI.

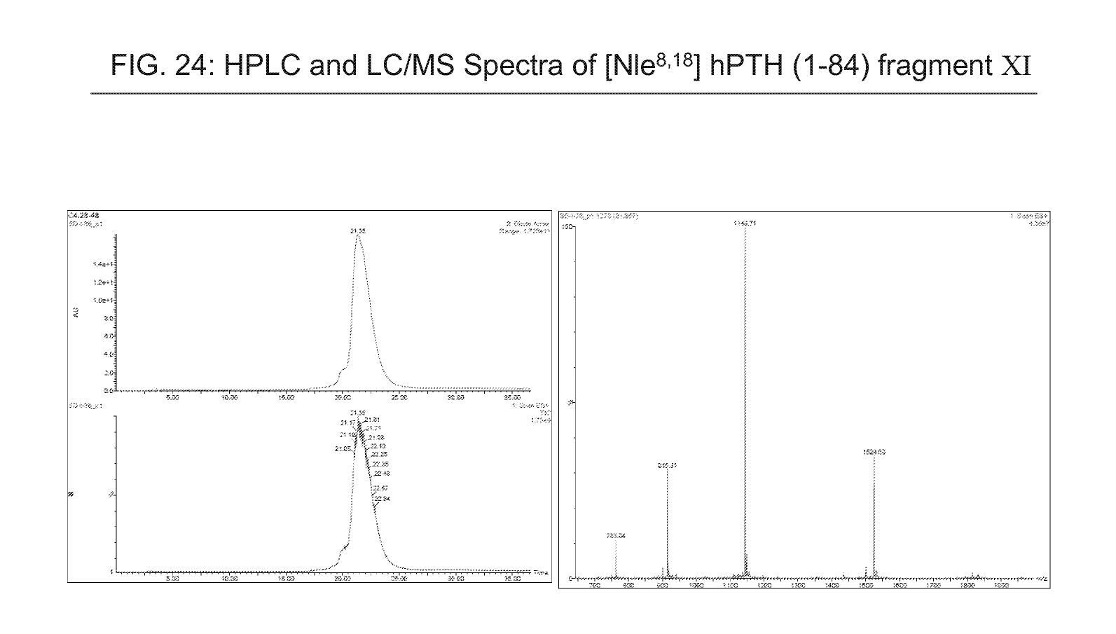

FIG. 25 presents HPLC and LC/MS spectra of [Nle^{8,18}]hPTH (1-84) fragment XIII

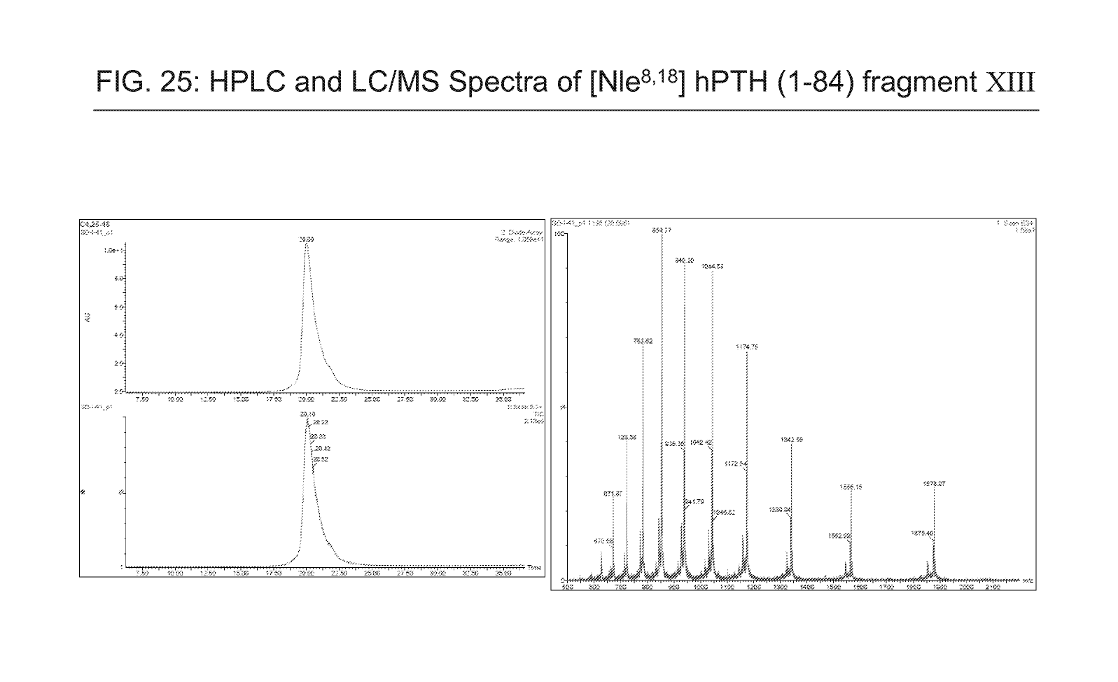

FIG. 26 presents HPLC and LC/MS spectra of [Nle^{8,18}]hPTH (1-37) fragment XIV.

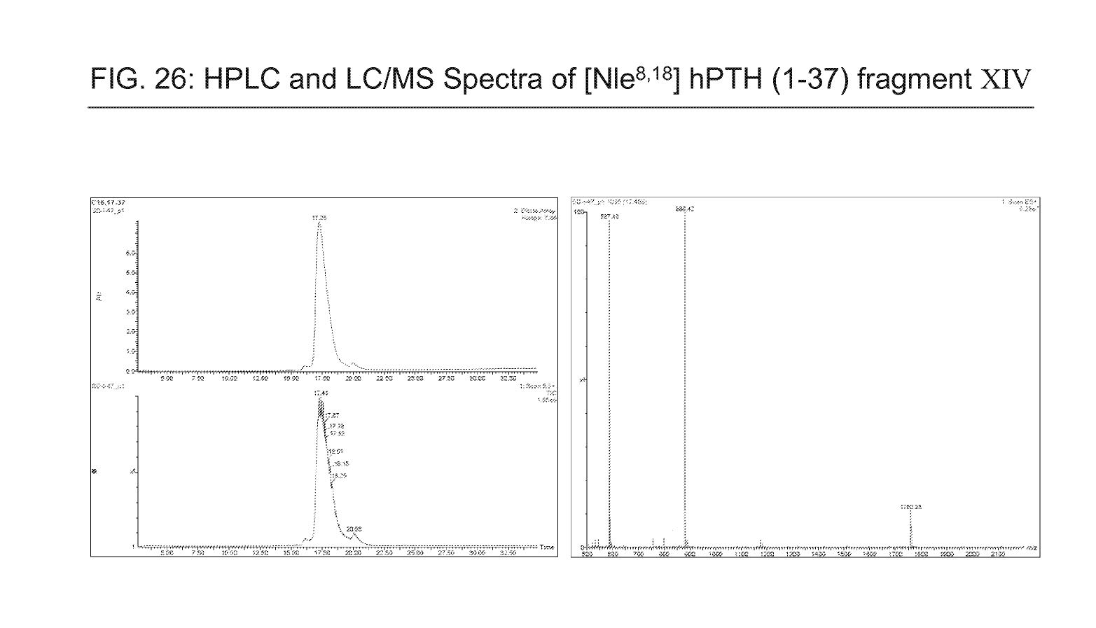

FIG. 27 presents HPLC and LC/MS spectra of [Nle^{8,18}]hPTH (1-37) fragment XV.

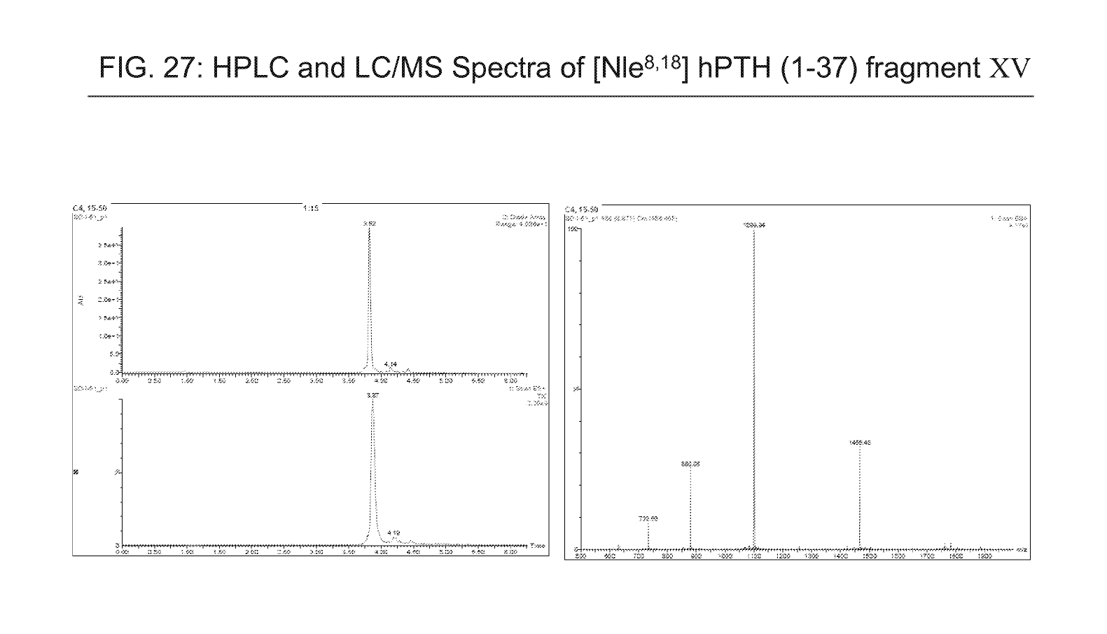

FIG. 28 presents HPLC and LC/MS spectra of [Nle^{8,18}]hPTH (1-37).

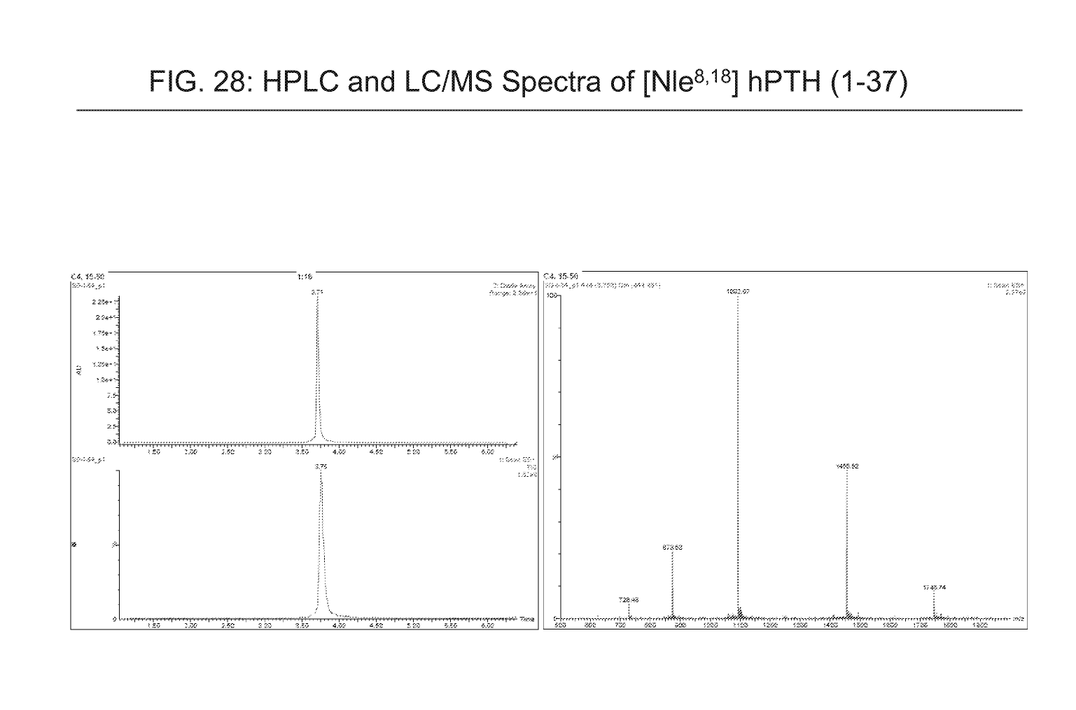

FIG. 29 depicts a three-dimensional representation of hPTH (1-39).

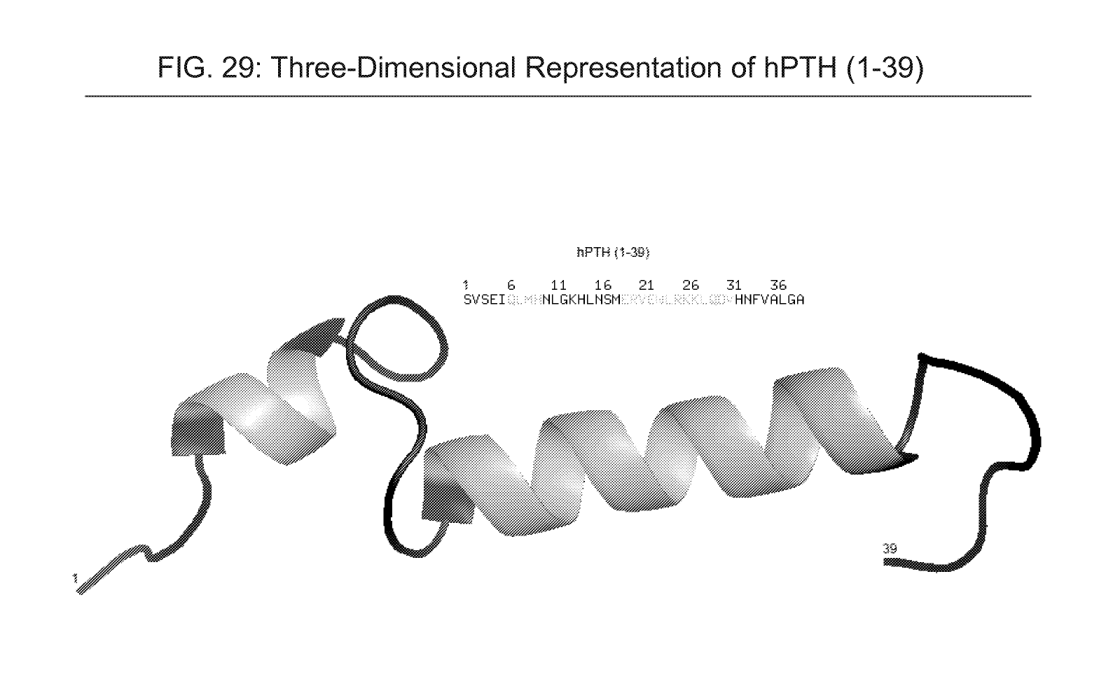

FIG. 30 presents HPLC and LC/MS spectra of hPTHrP (1-141) fragment XXX.

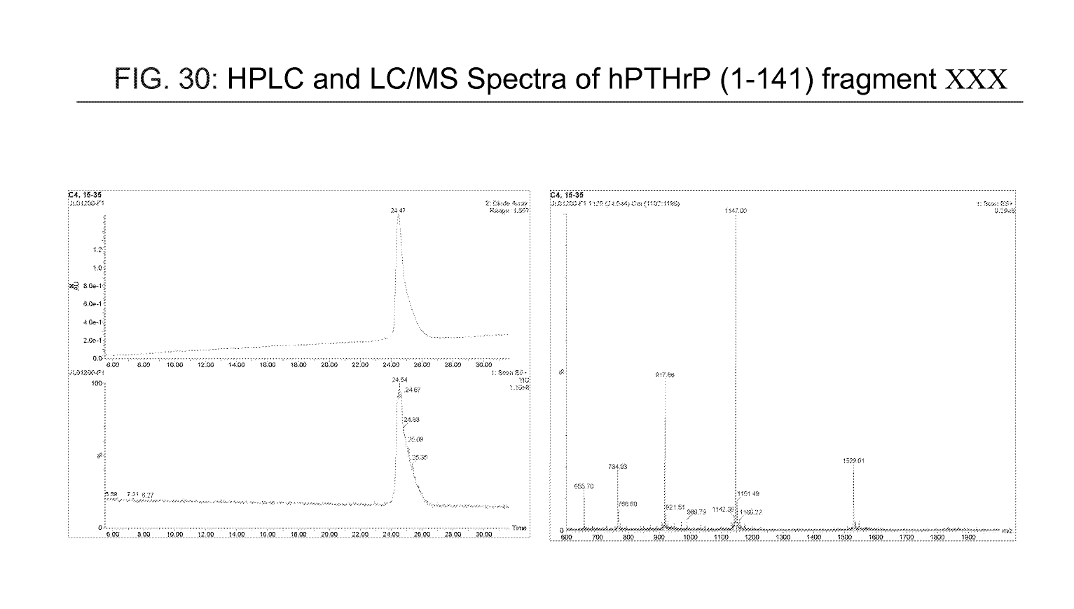

FIG. 31 presents HPLC and LC/MS spectra of hPTHrP (1-141) fragment XXXI.

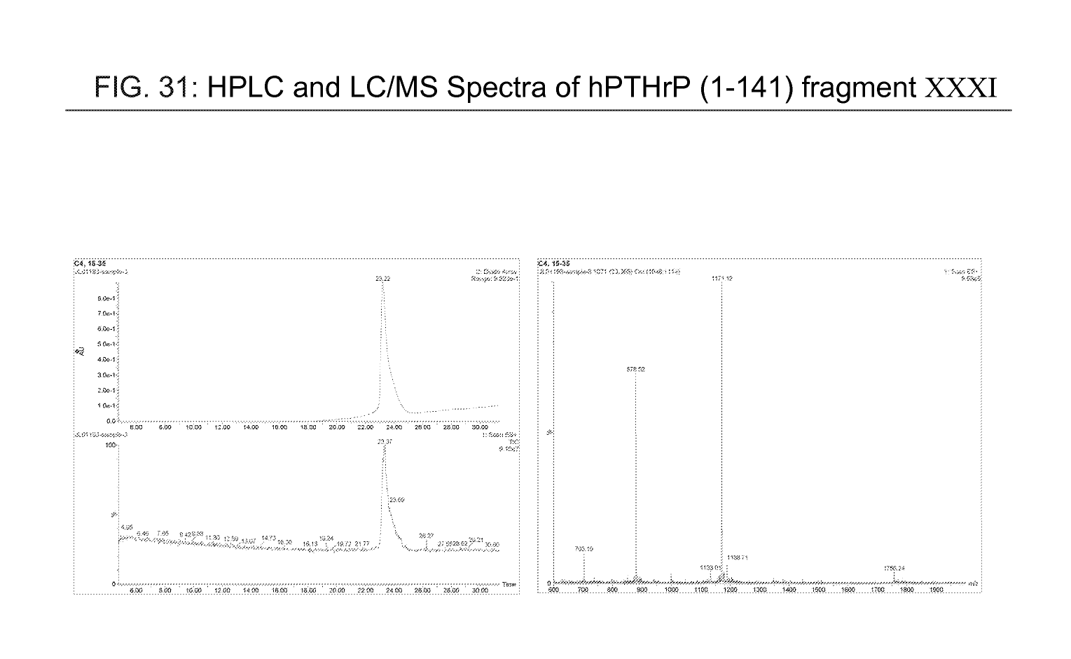

FIG. 32 presents HPLC and LC/MS spectra of hPTHrP (1-141) fragment XXXII.

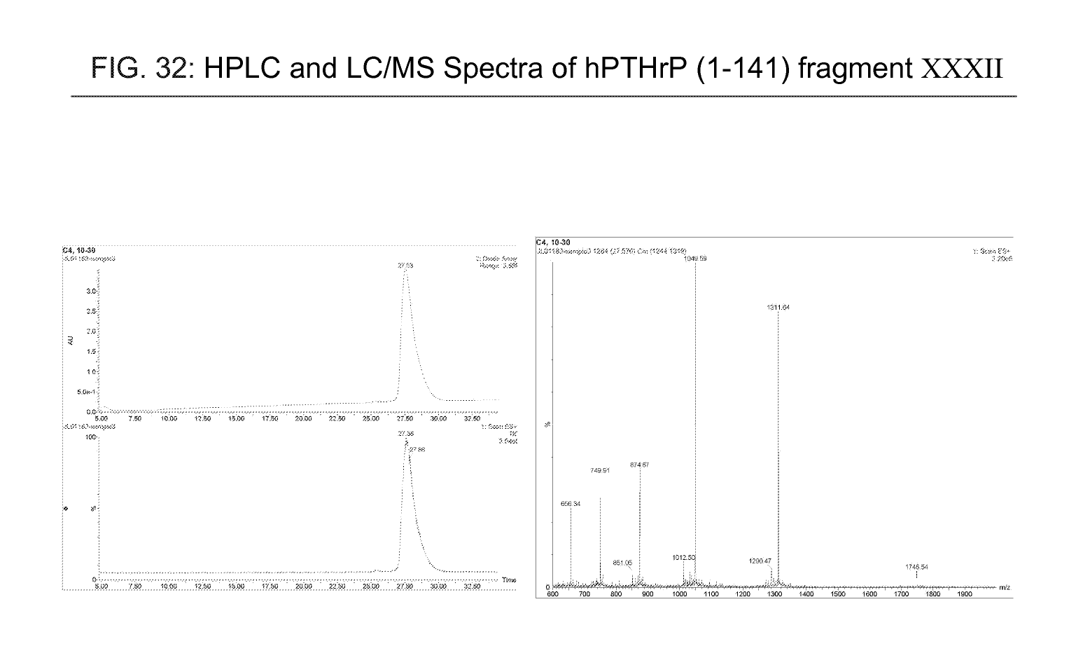

FIG. 33 presents HPLC and LC/MS spectra of hPTHrP (1-141) fragment XXXIII

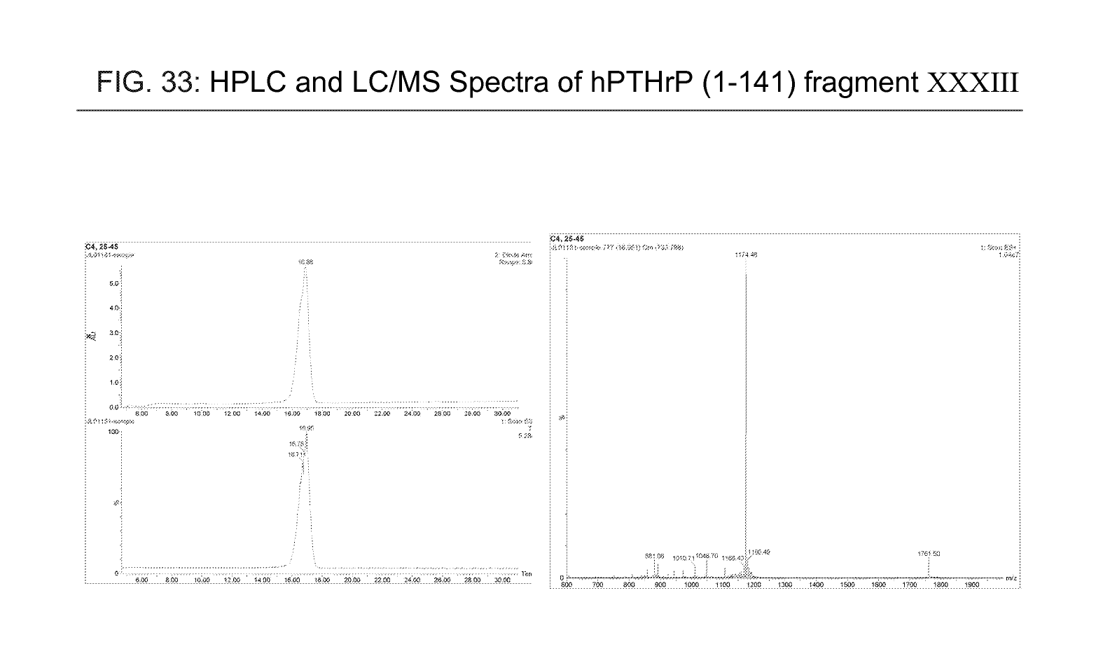

FIG. 34 presents HPLC and LC/MS spectra of hPTHrP (1-141) fragment XXXIV.

FIG. 35 presents HPLC and LC/MS spectra of hPTHrP (1-141) fragment XXXV.

FIG. 36 presents HPLC and LC/MS spectra of hPTHrP (1-141) fragment XXXVI.

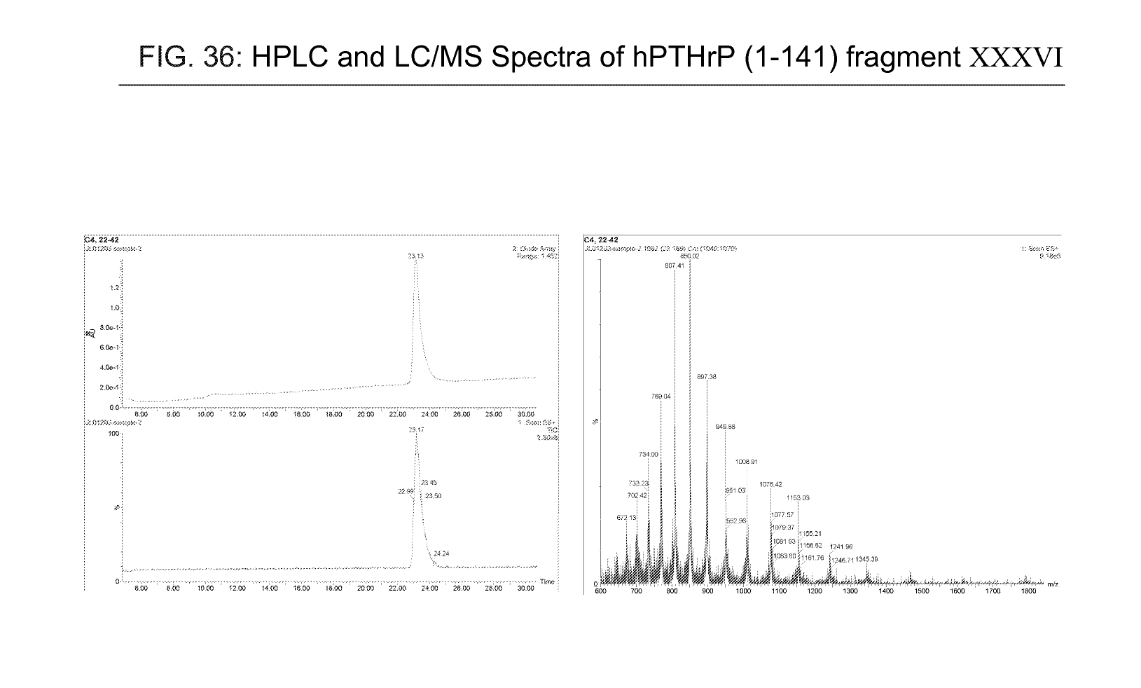

FIG. 37 presents HPLC and LC/MS spectra of hPTHrP (1-141) fragment XXXVII.

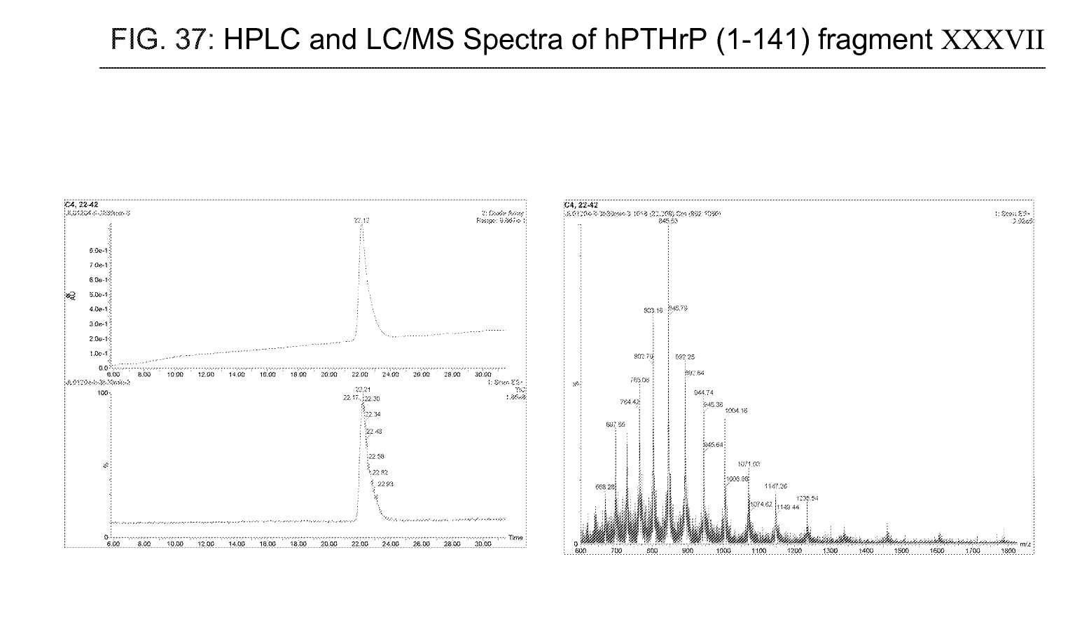

FIG. 38 depicts the stability of hPTH(1-84) after storage for seven (7) days.

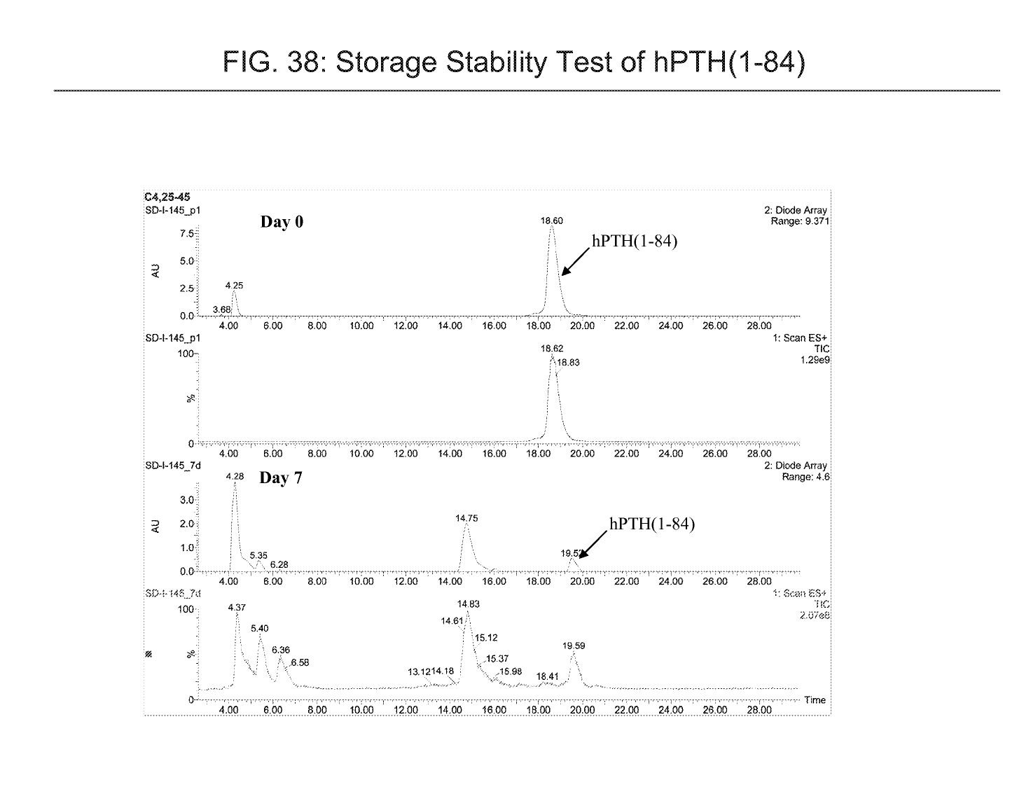

FIG. 39 depicts the stability of [Nle^{8,11}]hPTH(1-84) after storage for seven (7) days.

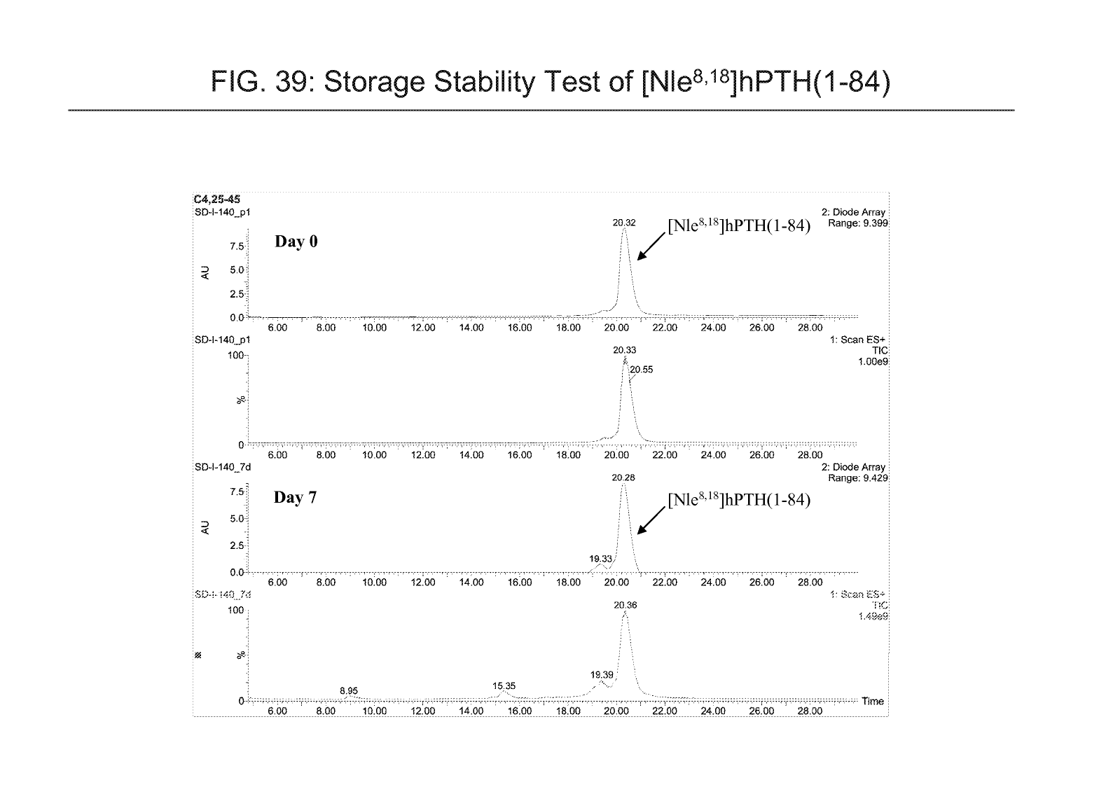

FIG. 40 depicts the stability of hPTH(1-37) after storage for seven (7) days.

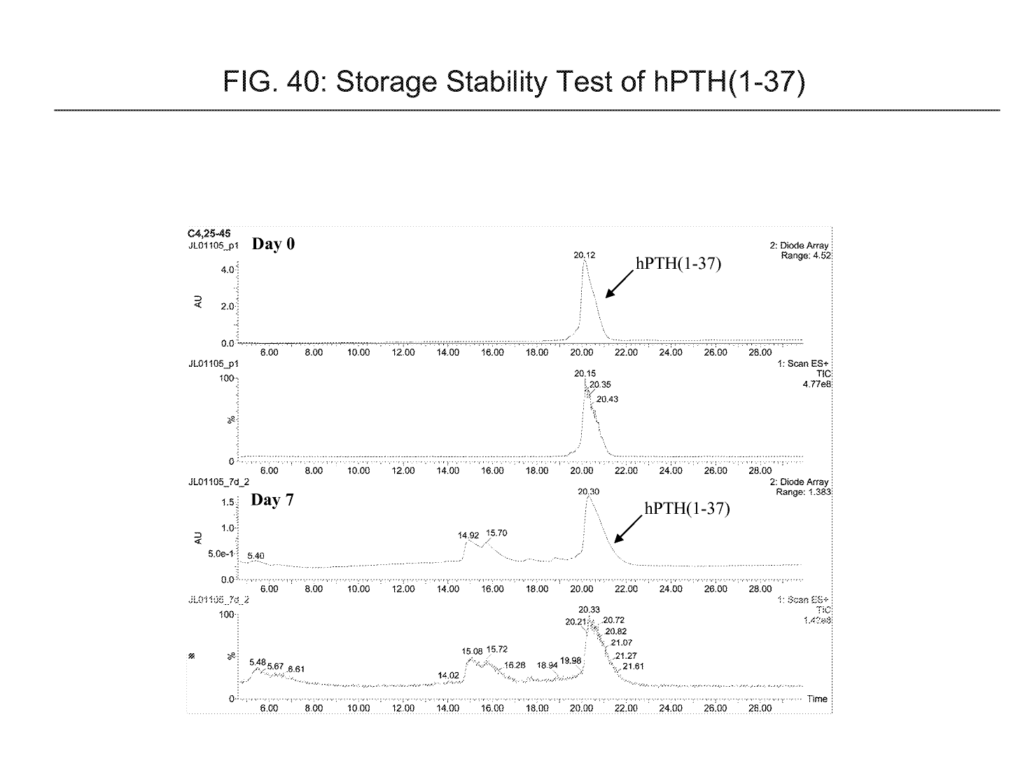

FIG. 41 depicts the stability of [Nle^{8,11}]hPTH(1-37) after storage for seven (7) days.

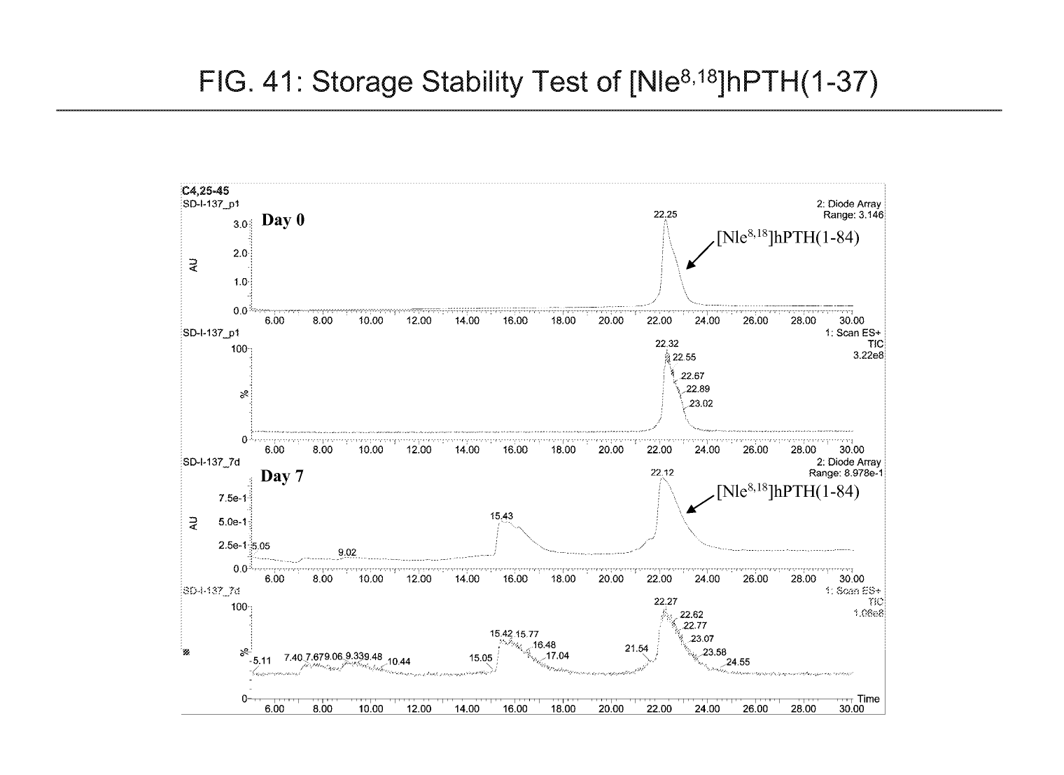

FIG. 42 depicts in vitro activity of hPTH analogs. The binding of PTH analogs were assessed in competition assays performed using membranes prepared from COS-7 cells transfected to express either the human PTHR1 in either the R⁰ (A) or RG (B) conformation, as described in Materials and Methods. cAMP assays were performed in HEK-293 cells transiently transfected to express the hPTHR1; intracellular cAMP was measured after ligand stimulation by radioimmunoassay (C) cAMP signaling was also assessed in cells co-transfected with a reporter plasmid encoding the luciferase gene under transcriptional control of a promoter containing a cAMP-response element (CRE-Luc), and measuring luminescence in response to varying concentrations of PTH analog (D). Data are means (±s.e.m.) of three experiments, each performed in duplicate. Assay parameters are reported in Table 1.

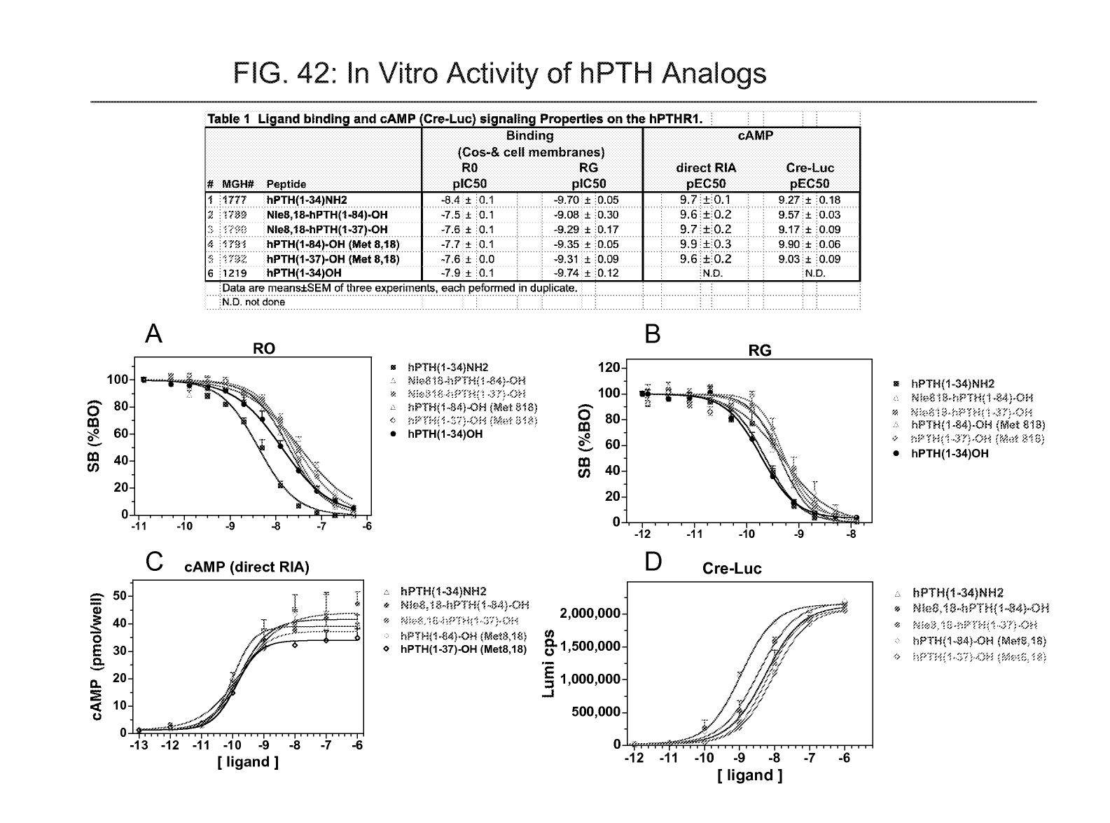

FIG. 43 depicts in vivo activity of hPTH analogs. Effects of PTH Analogs on Blood Ca⁺⁺ Levels in Mice. 9 week-old, male, C57BL/6 mice (total 32-35) were injected s.c. with vehicle or PTH analog (20 nmol/kg), and tail vein blood was collected at the indicated times thereafter (t=0 indicates blood collected immediately prior to injection, 1, 2, 4 or 6 hours post injection) and assessed for concentration of blood ionized Ca⁺⁺.

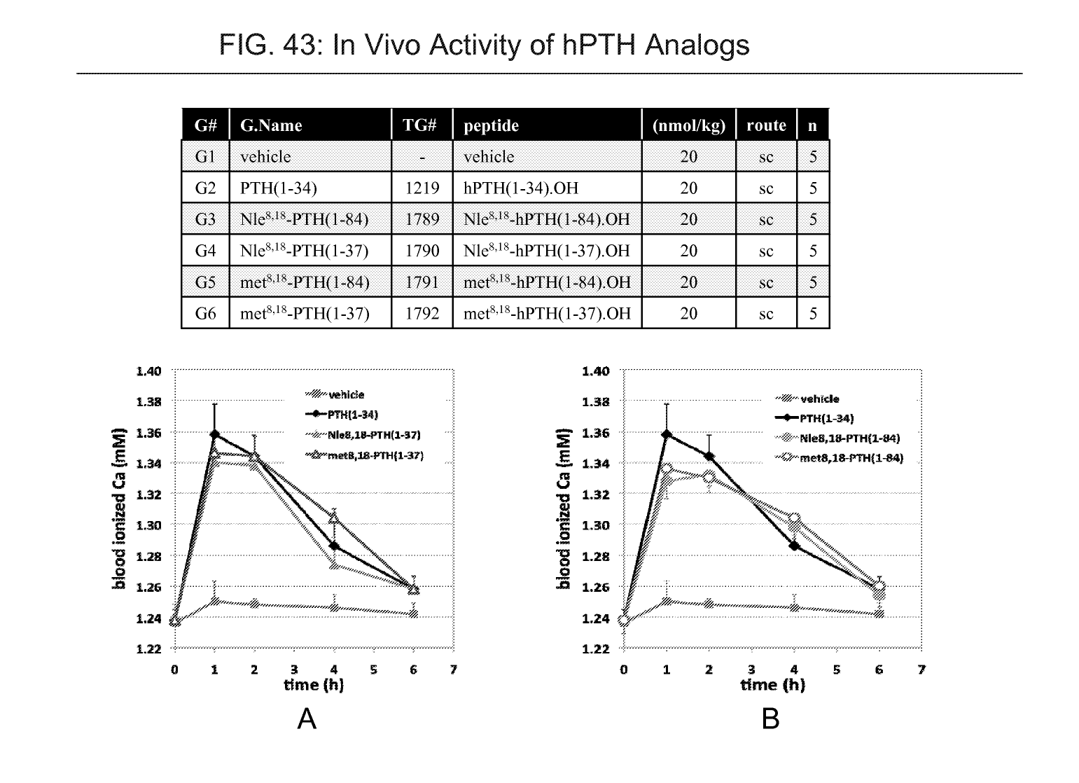

### DEFINITIONS

Biologically active. As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

Carrier. The term “carrier” refers to any chemical entity that can be incorporated into a composition containing an active agent (e.g., a peptide and/or analog of the present invention) without significantly interfering with the stability and/or activity of the agent (e.g., with a biological activity of the agent). In certain embodiments, the term “carrier” refers to a pharmaceutically acceptable carrier. An exemplary carrier herein is water.

Combination. As used herein, the term “combination,” “combined,” and related terms refers to a subject's simultaneous exposure to two or more therapeutic agents in accordance with this invention. For example, a compound of the present invention may be administered with another therapeutic agent simultaneously or sequentially in separate unit dosage forms or together in a single unit dosage form. Accordingly, the present invention provides, among other things, dosing regimens that involve administering at least a peptide of the present invention, an additional therapeutic agent, and a pharmaceutically acceptable carrier, adjuvant, or vehicle (the pharmaceutically acceptable carrier, adjuvant, or vehicle typically being in association with one or both of the peptide and the additional therapeutic agent.

Corresponding to. As used herein, the term “corresponding to” is often used to designate the position/identity of an amino acid residue in a parathyroid hormone peptide. Those of ordinary skill will appreciate that, for purposes of simplicity, a canonical numbering system (based on wild type hPTH—e.g., SEQ ID NO: 1) is utilized herein, so that an amino acid “corresponding to” a residue at position 19, for example, need not actually be the 19^{th} amino acid in a particular amino acid chain but rather corresponds to the residue found at position 19 in wild type hPTH; those of ordinary skill in the art readily appreciate how to identify corresponding amino acids.

Formulation. The term “formulation” refers to a composition that includes at least one active agent (e.g., a peptide and/or analog of the present invention) together with one or more carriers, excipients or other pharmaceutical additives for administration to a patient. In general, particular carriers, excipients and/or other pharmaceutical additives are selected in accordance with knowledge in the art to achieve a desired stability, release, distribution and/or activity of active agent(s) and which are appropriate for the particular route of administration.

Isolated. The term “isolated”, as used herein, refers to an agent or entity that has either (i) been separated from at least some of the components with which it was associated when initially produced (whether in nature or in an experimental setting); or (ii) produced by the hand of man. Isolated agents or entities may be separated from at least about 10%, 20%, 30%, 40%, 50%, 60%, 70%, 80%, 90%, or more of the other components with which they were initially associated. In some embodiments, isolated agents are more than 90%, 91%, 92%, 93%, 94%, 95%, 96%, 97%, 98%, 99% pure.

Non-natural amino acid. The phrase “non-natural amino acid” refers to an entity having the chemical structure of an amino acid (i.e.,:

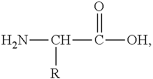

and therefore being capable of participating in at least two peptide bonds, but having an R group that differs from those found in amino acids in nature. In some embodiments, non-natural amino acids may also have a second R group rather than a hydrogen, and/or may have one or more other substitutions on the amino and/or carboxylic acid moieties. Non-limiting examples of a non-natural amino acid include norleucine (Nle), methoxinine (Mox), lanthionine, dehydroalanine, ornithine, citrulline, or 2-amino-isobutyric acid.

Parathyroid hormone analog: As described herein, a parathyroid hormone analog is a parathyroid hormone peptide whose amino acid sequence includes at least one point mutation as compared to wild type human parathyroid hormone. In some embodiments, a parathyroid hormone analog includes at least one non-natural amino acid residue as described herein.

Parathyroid hormone peptide: In general, as used herein, the term “parathyroid hormone peptide” refers to a polypeptide, or portion thereof that is at least about 3-85 amino acids long and shows an overall sequence identity of at least 80% with a corresponding portion of a wild type parathyroid hormone. In some embodiments, the overall sequence identity is ≧81%, ≧82%, ≧83%, ≧84%, ≧85%, ≧86%, ≧87%, ≧88%, ≧89%, ≧90%, ≧91%, ≧92%, ≧93%, ≧94%, ≧95%, ≧96%, ≧97%, ≧98%, ≧99% with a wild type parathyroid hormone. In many embodiments herein, the wild type parathyroid hormone is a wild type human parathyroid hormone, for example as set forth in SEQ ID NO: 1. In some embodiments, in addition to this overall sequence identity, a provided parathyroid hormone peptide includes one or more particular sequence elements, for example as described herein. In some embodiments, such a particular sequence element is an element that is characteristic of and/or conserved in parathyroid hormones in general or of certain subsets of parathyroid hormones. Particular embodiments of parathyroid hormone peptides are described in more detail herein below.

Parenteral. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Patient. The term “patient”, as used herein, means a mammal to which a formulation or composition comprising a formulation is administered, and in some embodiments includes humans.

Pharmaceutically acceptable carrier, adjuvant, or vehicle. The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Polypeptide. A “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain.

Pure. As used herein, an agent or entity is “pure” if it is substantially free of other components. For example, a preparation that contains more than about 90% of a particular agent or entity is typically considered to be a pure preparation. In some embodiments, an agent or entity is at least 91%, 92%, 93%, 94%, 95%, 96%, 97%, 98%, or 99% pure.

Therapeutic agent. As used herein, the phrase “therapeutic agent” refers to any agent that elicits a desired biological or pharmacological effect when administered to an organism.

Therapeutically effective amount and effective amount. As used herein, and unless otherwise specified, the terms “therapeutically effective amount” and “effective amount” of an agent refer to an amount sufficient to provide a therapeutic benefit in the treatment, prevention and/or management of a disease, disorder, or condition, e.g., to delay onset of or minimize (e.g., reduce the incidence and/or magnitude of) one or more symptoms associated with the disease, disorder or condition to be treated. In some embodiments, a composition may be said to contain a “therapeutically effective amount” of an agent if it contains an amount that is effective when administered as a single dose within the context of a therapeutic regimen. In some embodiments, a therapeutically effective amount is an amount that, when administered as part of a dosing regimen, is statistically likely to delay onset of or minimize (reduce the incidence and/or magnitude of) one or more symptoms or side effects of a disease, disorder or condition. In some embodiments, a “therapeutically effective amount” is an amount that enhances therapeutic efficacy of another agent with which the composition is administered in combination. In some embodiments, a therapeutically effective amount for administration to a human corresponds to a reference amount (e.g., a therapeutically effective amount in an animal model such as a mouse model) adjusted for body surface area of a human as compared with body surface area of the animal model, as is known in the art (see, for example Reagan-Shaw et al., “Dose translation from animal to human studies revisited,” The FASEB Journal 22: 659-661 (2007), the entirety of which is herein incorporated by reference). In some embodiments, the reference therapeutically effective amount is an amount that is therapeutically effective in a mouse model, for example, as described herein. In some embodiments, the reference therapeutically effective amount is within the range of about 0.0001 mg/kg to about 500 mg/kg. In some embodiments, the reference therapeutically effective amount is within the range of about 0.0001 mg/kg to about 0.001 mg/kg. In some embodiments, the reference therapeutically effective amount is within the range of about 0.001 mg/kg to about 0.01 mg/kg. In some embodiments, the reference therapeutically effective amount is within the range of about 0.01 mg/kg to about 0.1 mg/kg. In some embodiments, the reference therapeutically effective amount is within the range of about 0.1 mg/kg to about 0.5 mg/kg. In some embodiments, the reference therapeutically effective amount is within the range of about 0.5 mg/kg to about 1 mg/kg. In some embodiments, the reference therapeutically effective amount is within the range of about 1 mg/kg to about 2.5 mg/kg. In some embodiments, the reference therapeutically effective amount is within the range of about 2.5 mg/kg to about 10 mg/kg. In some embodiments, the reference therapeutically effective amount is within the range of about 10 mg/kg to about 50 mg/kg. In some embodiments, the reference therapeutically effective amount is within the range of about 50 mg/kg to about 100 mg/kg. In some embodiments, the reference therapeutically effective amount is within the range of about 100 mg/kg to about 250 mg/kg. In some embodiments, the reference therapeutically effective amount is within the range of about 250 mg/kg to about 500 mg/kg. hPTH is currently administered at a dose of 20 micrograms (mcg) per day. In some embodiments, the therapeutically effective amount of peptides and/or analogs of the present invention is within a range of 0.1-50 mcg per day. In some embodiments, the therapeutically effective amount of peptides and/or analogs of the present invention is within a range of 10-100 mcg per day.

Treat or Treating. The terms “treat” or “treating,” as used herein, refer to partially or completely alleviating, inhibiting, delaying onset of, reducing the incidence of, yielding prophylaxis of, ameliorating and/or relieving a disorder, disease, or condition, or one or more symptoms or manifestations of the disorder, disease or condition.

Unit Dose. The expression “unit dose” as used herein refers to a physically discrete unit of a formulation appropriate for a subject to be treated (e.g., for a single dose); each unit containing a predetermined quantity of an active agent selected to produce a desired therapeutic effect when administered according to a therapeutic regimen (it being understood that multiple doses may be required to achieve a desired or optimum effect), optionally together with a pharmaceutically acceptable carrier, which may be provided in a predetermined amount. The unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may contain a variety of components in addition to the therapeutic agent(s). For example, acceptable carriers (e.g., pharmaceutically acceptable carriers), diluents, stabilizers, buffers, preservatives, etc., may be included as described infra. It will be understood, however, that the total daily usage of a formulation of the present invention will be decided by the attending physician within the scope of sound medical judgment. The specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Useful Cysteine or Methionine Residue. As used herein, the term “useful” or “useful cysteine and/or methionine residue” refers to a residue that is located at a position which enables the synthesis of targeted peptides or proteins. “Useful” cysteine and/or methionine residues permit the synthesis of moderately-sized fragments (>15 amino acids or <50 amino acids long). “Useful” cysteine and/or methionine residues are residues which are not located on the N-terminal side of unfavorable amino acids such as isoleucine (Ile), valine (Val), threonine (Thr) and proline (Pro). A person of ordinary skill in the art would immediately recognize such “useful” cysteine and/or methionine residues.

Wild type. As is understood in the art, the phrase “wild type” generally refers to a normal form of a protein or nucleic acid, as is found in nature.

### DETAILED DESCRIPTION OF CERTAIN EMBODIMENTS

Parathyroid Hormone Peptides

Human Parathyroid Hormone (hPTH) is a biological messenger that is secreted by the parathyroid glands as a peptide containing 84-amino acids. (Potts J T. 2005. “Parathyroid hormone: past and present.” J. Endocrinol. 187: 311-25; Potts J T, Gardella T J. 2007. “Progress, paradox, and potential: parathyroid hormone research over five decades.” Ann. NY Acad. Sci. 1117: 196-208). By binding to its receptor, hPTH can enhance the concentration of calcium (Ca²) in the blood. (Talmage R V, Mobley H T. 2008. “Calcium homeostasis: reassessment of the actions of parathyroid hormone.” Gen. Comp. Endocrinol. 156: 1-8). Because of its important physiological role, the fragment hPTH (1-34) is now given by subcutaneous injection for the treatment of hypoparathyroidism and osteoporosis in men and post-menopausal women who are at high risk for fracture. (Dominguez L J, Scalisi R, Barbagallo M. 2010. “Therapeutic options in osteoporosis.” Acta Biomed. 81 Suppl 1: 55-65; Ellegaard M, Jorgensen N R, Schwarz P. 2010. “Parathyroid hormone and bone healing.” Calcif. Tissue Int. 87: 1-13; Fraser W D. 2009. “Hyperparathyroidism.” Lancet 374: 145-58).

Like most hormone drugs, the recombinant hPTH therapeutics have very short half-lives in the human body and need to be taken at least once a day. (Bieglmayer C, Prager G, Niederle B. 2002. “Kinetic analyses of parathyroid hormone clearance as measured by three rapid immunoassays during parathyroidectomy.” Clin. Chem. 48: 1731-18; Abraham A K, Mager D E, Gao X, Li M, Healy D R, Maurer T S. 2009. “Mechanism-based pharmacokinetic/pharmacodynamic model of parathyroid hormone-calcium homeostasis in rats and humans.” J. Pharmacol. Exp. Ther. 330: 169-78). The need for continuous daily subcutaneous injection is a distinct disadvantage and has limited the use of the hormone. In addition, it can cause discomfort and may lead to long-term complications, especially to patients with already established and severe osteoporosis. Therefore, the production of more stable forms of hPTH is desirable. (Potts J T, Jr., Gardella T J, Juppner H, Kronenberg H M. 1997. “Structure based design of parathyroid hormone analogs.” J. Endocrinol. 154 Suppl: S15-21; Reissmann S, Imhof D. 2004. “Development of conformationally restricted analogues of bradykinin and somatostatin using constrained amino acids and different types of cyclization.” Curr. Med. Chem. 11: 2823-44). Accordingly, there exists a need for more stable and efficacious analogs of hPTH.

In some embodiments, the present invention encompasses the recognition that increasing the stability and half-life of hPTH and/or hPTHrP therapies facilitates more tolerable administration and greater patient compliance. In some embodiments, the present invention provides stable hPTH therapeutics. In some embodiments, provided hPTH analogs have greater stability than hPTH of SEQ ID NO: 2 (e.g., when measured in an in vitro peptide stability assay in human serum).

In certain embodiments, the present invention provides a human parathyroid hormone (hPTH) peptide and/or analog.

A full length, wild type hPTH sequence is depicted by SEQ ID NO: 1. In some embodiments, a parathyroid hormone peptide and/or analog has an amino acid sequence that is overall ≧80%, ≧81%, ≧82%, ≧83%, ≧84%, ≧85%, ≧86%, ≧87%, ≧88%, ≧89%, ≧90%, ≧91%, ≧92%, ≧93%, ≧94%, ≧95%, ≧96%, ≧97%, ≧98%, ≧99% or more identical to SEQ ID NO: 1 or SEQ ID NO: 2.

In some embodiments, a parathyroid hormone peptide and/or analog has an amino acid sequence that is overall ≧80%, ≧81%, ≧82%, ≧83%, ≧84%, ≧85%, ≧86%, ≧87%, ≧88%, ≧89%, ≧90%, ≧91%, ≧92%, ≧93%, ≧94%, ≧95%, ≧96%, ≧97%, ≧98%, ≧99% or more identical to SEQ ID NO: 6 or SEQ ID NO: 7.

In some embodiments, a parathyroid hormone peptide and/or analog has an amino acid sequence that is overall ≧80%, ≧81%, ≧82%, ≧83%, ≧84%, ≧85%, ≧86%, ≧87%, ≧88%, ≧89%, ≧90%, ≧91%, ≧92%, ≧93%, ≧94%, ≧95%, ≧96%, ≧97%, ≧98%, ≧99% or more identical to SEQ ID NO: 14 or SEQ ID NO: 15.

In certain embodiments, the present invention provides a parathyroid hormone peptide and/or analog 3-84 amino acids in length. In some embodiments, provided parathyroid hormone peptides and/or analogs have an amino acid sequence that is at least a minimum length and not more than a maximum length, wherein the minimum length is, for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or more amino acids, and where the maximum length is not more than 84, 83, 82, 81, 80, 79, 78, 77, 76, 75, 74, 73, 72, 71 or 70 amino acids in length.

In some embodiments, a provided parathyroid hormone peptide and/or analog is 84-amino acids in length.

In certain embodiments, a provided parathyroid hormone peptide and/or analog is 34-amino acids in length.

In some embodiments, a provided parathyroid hormone peptide and/or analog is 37-amino acids in length.

In some embodiments, a provided parathyroid hormone peptide and/or analog is 39-amino acids in length.

In some embodiments, a provided parathyroid hormone peptide and/or analog includes at least one non-natural amino acid residue selected from the group consisting of norleucine, methoxinine, and combinations thereof. In some embodiments, a provided parathyroid hormone peptide and/or analog includes a non-natural amino acid at a position corresponding to residue 8 and/or residue 18 in SEQ ID NO: 1 or SEQ ID NO: 2. In some embodiments, a provided parathyroid hormone peptide and/or analog includes at least one non-natural amino acid at a position corresponding to residue 8 and/or residue 18 in SEQ ID NO: 1 or SEQ ID NO: 2. In some embodiments, a provided parathyroid hormone peptide and/or analog includes a non-natural amino acid at a position corresponding to residue 8 in SEQ ID NO: 1 or SEQ ID NO: 2. In some embodiments, a provided parathyroid hormone peptide and/or analog includes a non-natural amino acid at a position corresponding to residue 18 in SEQ ID NO: 1 or SEQ ID NO: 2. In some embodiments, a provided parathyroid hormone peptide and/or analog includes two non-natural amino acid at the positions corresponding to residue 8 and residue 18 in SEQ ID NO: 1 or SEQ ID NO: 2.

In some embodiments, a provided parathyroid hormone peptide and/or analog includes a non-natural amino acid at one or more positions corresponding to residues X₁, X₇, X₈, X₁₆, X₁₈, X₂₁, X₂₂, X₂₆, X₃₅, X₃₆, X₃₉, X₄₀, X₄₁, X₄₂, X₄₃, X₄₅, X₄₆, X₄₇, X₄₈, X₅₂, X₅₆, X₅₈, X₅₉, X₆₀, X₆₁, X₆₂, X₆₃, X₆₄, X₇₀, X₇₄, X₇₆, X₇₉, X₈₁ or X₈₃ of SEQ ID NO: 2.

SEQ ID NO: 3, SEQ ID NO: 4 and SEQ ID NO: 5 depict conserved sequence elements found in wild type parathyroid hormone peptides in various species. In some embodiments, a parathyroid hormone peptide and/or analog includes at least one of SEQ ID NO: 3, SEQ ID NO: 4 and SEQ ID NO: 5.

In some embodiments, a parathyroid hormone peptide and/or analog has an amino acid sequence which includes an element ≧79%, ≧82%, ≧85%, ≧88%, ≧91%, ≧94% or ≧97% identical to SEQ ID NO: 6.

In some embodiments, a parathyroid hormone peptide and/or analog has an amino acid sequence which includes an element ≧79%, ≧82%, ≧85%, ≧88%, ≧91%, ≧94% or ≧97% identical to SEQ ID NO: 7.

Glycosylated Parathyroid Hormone Peptides. Glycosylation is a common post-translational modification known to affect the characteristics of peptides and proteins. In particular, glycosylation can affect the folding, stability and function of peptides and proteins. However, while peptide sequences can be recombinantly expressed in biological systems, producing biosynthetic glycopeptides with high specificity remains difficult. More specifically, glycosylation in biological systems results in a composition which is a) not uniform and b) variable, so that particular purification steps are needed to obtain a homogenous preparation. In contrast, the chemical synthesis of peptides and/or analogs of the present invention allows for precise incorporation of specific or particular glycans into a peptide sequence.

Peptides may be glycosylated by any one of several methods known to a person of ordinary skill in the art. More particularly, an amino acid is glycosylated before being incorporated into the peptide. In some embodiments, the present invention provides a parathyroid hormone peptide and/or analog glycosylated with at least one glycan group.

In some embodiments, the at least one glycan group is selected from:

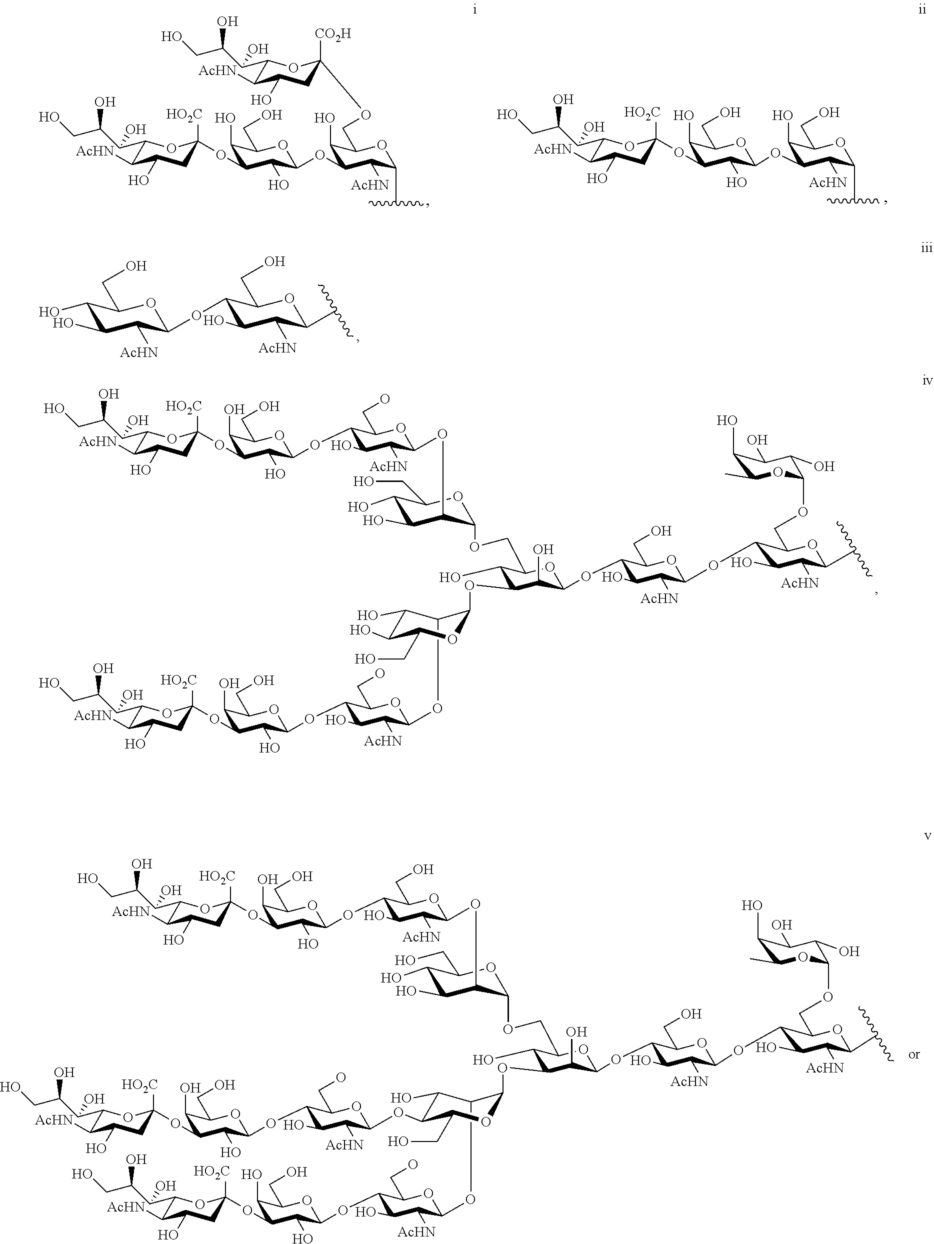

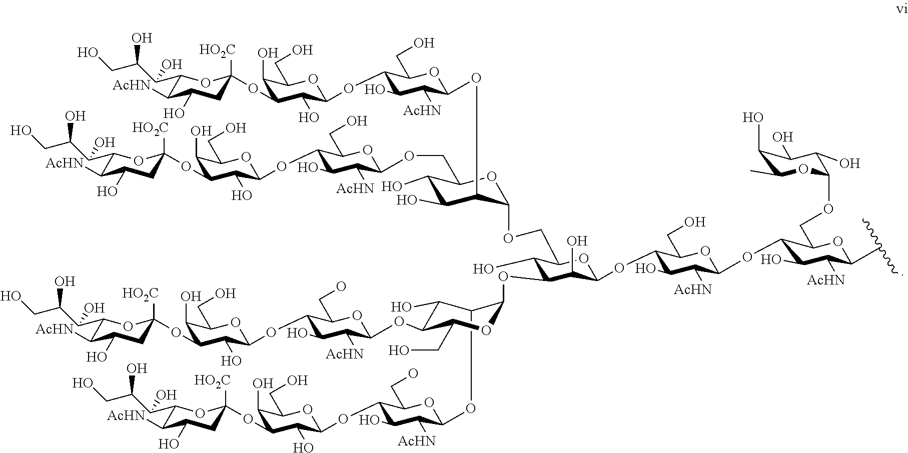

In some embodiments, a provided parathyroid hormone peptide and/or analog is O-glycosylated. In some embodiments, a provided parathyroid hormone peptide and/or analog is glycosylated at one or more serine or threonine residues. In some embodiments, a provided parathyroid hormone peptide and/or analog is O-glycosylated with a glycan selected from:

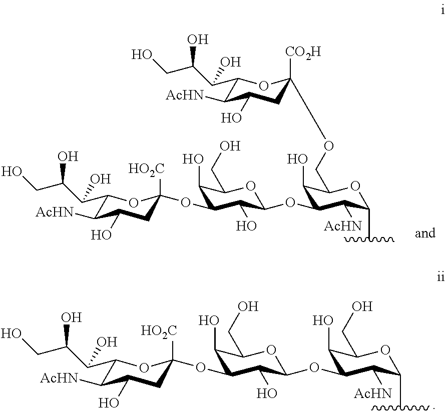

In some embodiments, a parathyroid hormone peptide and/or analog is glycosylated at S₁.

In some embodiments, a parathyroid hormone peptide and/or analog is N-glycosylated. In certain embodiments, a provided parathyroid hormone peptide and/or analog is glycosylated at one or more asparagine or glutamine residues. In some embodiments, a parathyroid hormone peptide and/or analog is N-glycosylated with a glycan selected from:

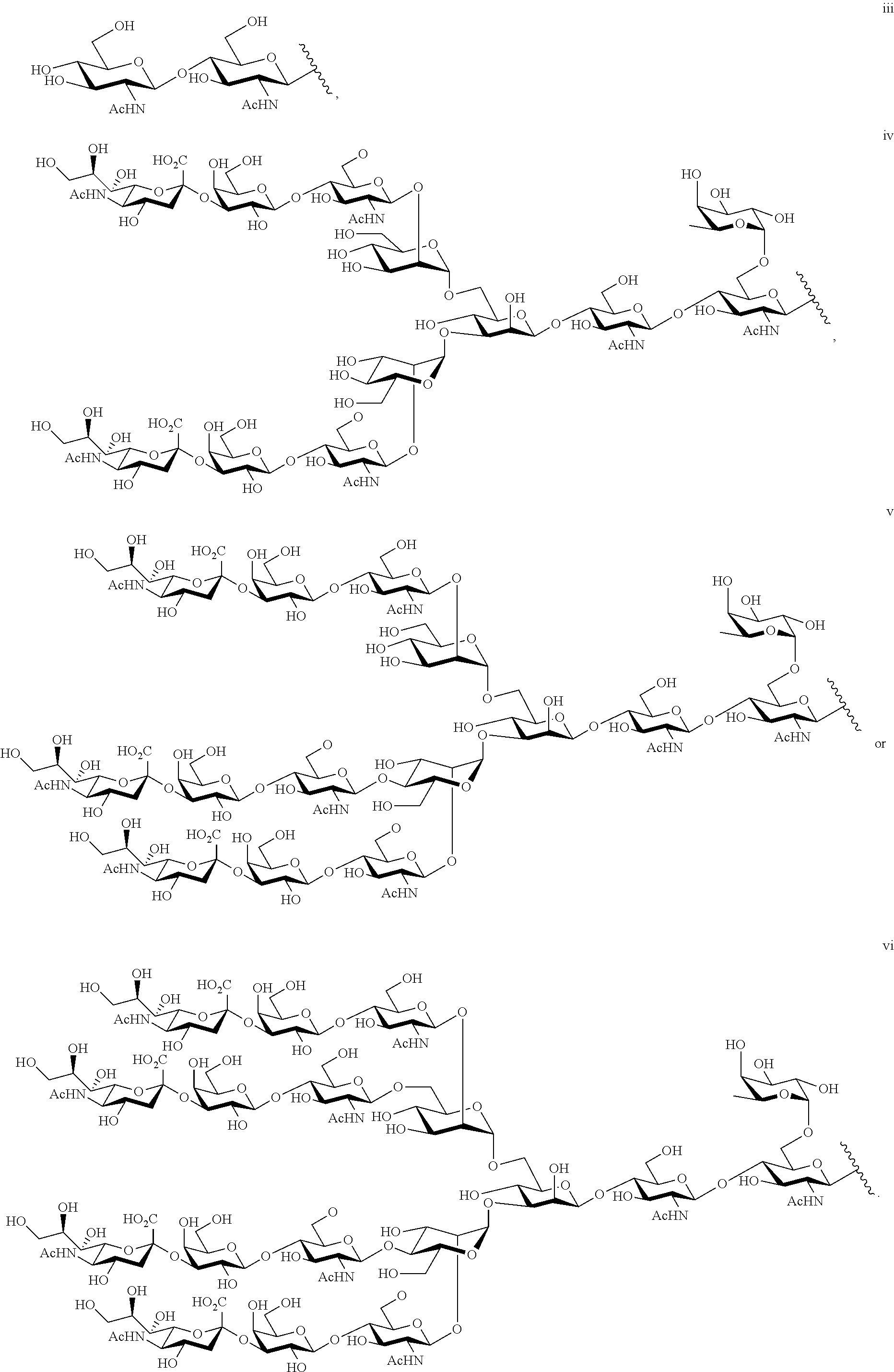

In some embodiments, a parathyroid hormone peptide and/or analog is glycosylated at N₃₃.

Particular PTH Peptides and/or Analogs

One of ordinary skill in the art reading the present disclosure will appreciate that, in certain embodiments, provided hPTH peptides and/or analogs are characterized by two or more features as are discussed individually above. For example, in certain embodiments, a provided hPTH peptide and/or analog has an amino acid sequence ≧80% identical to SEQ ID NO: 1 or SEQ ID NO: 2, wherein the parathyroid hormone peptide and/or analog includes at least one non-natural amino acid. In some such embodiments, the at least one non-natural amino acid is selected from the group consisting of norleucine and/or methoxinine.

In some embodiments, a provided hPTH peptide and/or analog has an amino acid sequence ≧80% identical to SEQ ID NO: 2, wherein the parathyroid hormone peptide and/or analog includes at least one non-natural amino acid at a position corresponding to residue 8 and/or residue 18 in SEQ ID NO: 2. In some such embodiments, the at least one non-natural amino acid is selected from the group consisting of norleucine and/or methoxinine.

In some embodiments, a provided hPTH peptide and/or analog has a sequence 84-amino acids in length, wherein the amino acid sequence includes at least one non-natural amino acid. In some embodiments, a provided hPTH peptide and/or analog has a sequence 84-amino acids in length, wherein the amino acid sequence includes at least one non-natural amino acid selected from norleucine, methoxinine and combinations thereof.

In some embodiments, a provided hPTH peptide and/or analog has a sequence 37-amino acids in length, wherein the amino acid sequence includes at least one non-natural amino acid. In some embodiments, a provided hPTH peptide and/or analog has a sequence 37-amino acids in length, wherein the amino acid sequence includes at least one non-natural amino acid selected from norleucine, methoxinine and combinations thereof.

In some embodiments, a provided hPTH peptide and/or analog has a sequence 39-amino acids in length, wherein the amino acid sequence includes at least one non-natural amino acid. In some embodiments, a provided hPTH peptide and/or analog has a sequence 39-amino acids in length, wherein the amino acid sequence includes at least one non-natural amino acid selected from norleucine, methoxinine and combinations thereof.

In some embodiments, a provided hPTH peptide and/or analog has a sequence 34-amino acids in length, wherein the amino acid sequence includes at least one non-natural amino acid. In some embodiments, a provided hPTH peptide and/or analog has a sequence 34-amino acids in length, wherein the amino acid sequence includes at least one non-natural amino acid selected from norleucine, methoxinine and combinations thereof.

In some embodiments, a provided hPTH peptide and/or analog has an amino acid sequence ≧80%, ≧85%, ≧90% or ≧95% identical to SEQ ID NO: 1 or SEQ ID NO: 2 and includes at least one of SEQ ID NO: 3, SEQ ID NO: 4 and SEQ ID NO: 5.

In some embodiments, a provided parathyroid hormone peptide and/or analog has an amino acid sequence ≧80%, ≧85%, ≧90% or ≧95% identical to SEQ ID NO: 2, wherein X₁ is S or A; X₇ is F or L; X₁₆ is N, S or A; X₁₈ is M, L or V; X₂₁ is V or M; and X₂₂ is E or Q. In some embodiments, a parathyroid hormone peptide and/or analog has an amino acid sequence ≧80%, ≧85%, ≧90% or ≧95% identical to SEQ ID NO: 2, wherein X₁ is S, A, Nle or Mox; X₇ is F, L, Nle or Mox; X₁₆ is N, S, A, Nle or Mox; X₁₈ is M, L, V, Nle or Mox; X₂₁ is V, M, Nle or Mox; and X₂₂ is E, Q, Nle or Mox.

In some embodiments, a parathyroid hormone peptide and/or analog has an amino acid sequence ≧80%, ≧85%, ≧90% or ≧95% identical to SEQ ID NO: 2, wherein at least one of X₃₆ is A, Nle or Mox; X₃₉ is A, Nle or Mox; X₄₅ is D, Nle or Mox; X₄₈ is S, Nle or Mox; X₅₆ is D, Nle or Mox; X₅₈ is V, Nle or Mox; X₆₀ is V, Nle or Mox; X₆₁ is E, Nle or Mox; X₆₂ is E, Nle or Mox; X₇₀ is A, Nle or Mox; X₇₄ is D, Nle or Mox; and X₈₁ is A, Nle or Mox.

In some embodiments, a parathyroid hormone peptide and/or analog has an amino acid sequence which is ≧94% identical to SEQ ID NO: 14, wherein residues corresponding to positions 8 and 18 are selected from the group consisting of methionine, methoxinine, norleucine, and combinations thereof.

In some embodiments, a parathyroid hormone peptide and/or analog has an amino acid sequence which is ≧94% identical to SEQ ID NO: 14, wherein the residues corresponding to positions 8 and 18 are selected from the group consisting of methionine, methoxinine, norleucine, and combinations thereof, with the proviso that residues corresponding to positions 8 and 18 are not both norleucine.

In some embodiments, a parathyroid hormone peptide and/or analog has an amino acid sequence which is ≧94% identical to SEQ ID NO: 14, wherein the residues corresponding to positions 8 and 18 are selected from the group consisting of methionine, methoxinine, norleucine, and combinations thereof, with the proviso that residues corresponding to positions 8 and 18 are not both methionine.

In some embodiments, a parathyroid hormone peptide and/or analog has an amino acid sequence which is ≧80%, ≧85%, ≧90% or ≧95% identical to SEQ ID NO: 1 or SEQ ID NO: 2 and is glycosylated with at least one glycan group. In some such embodiments, the at least one glycan group is selected from:

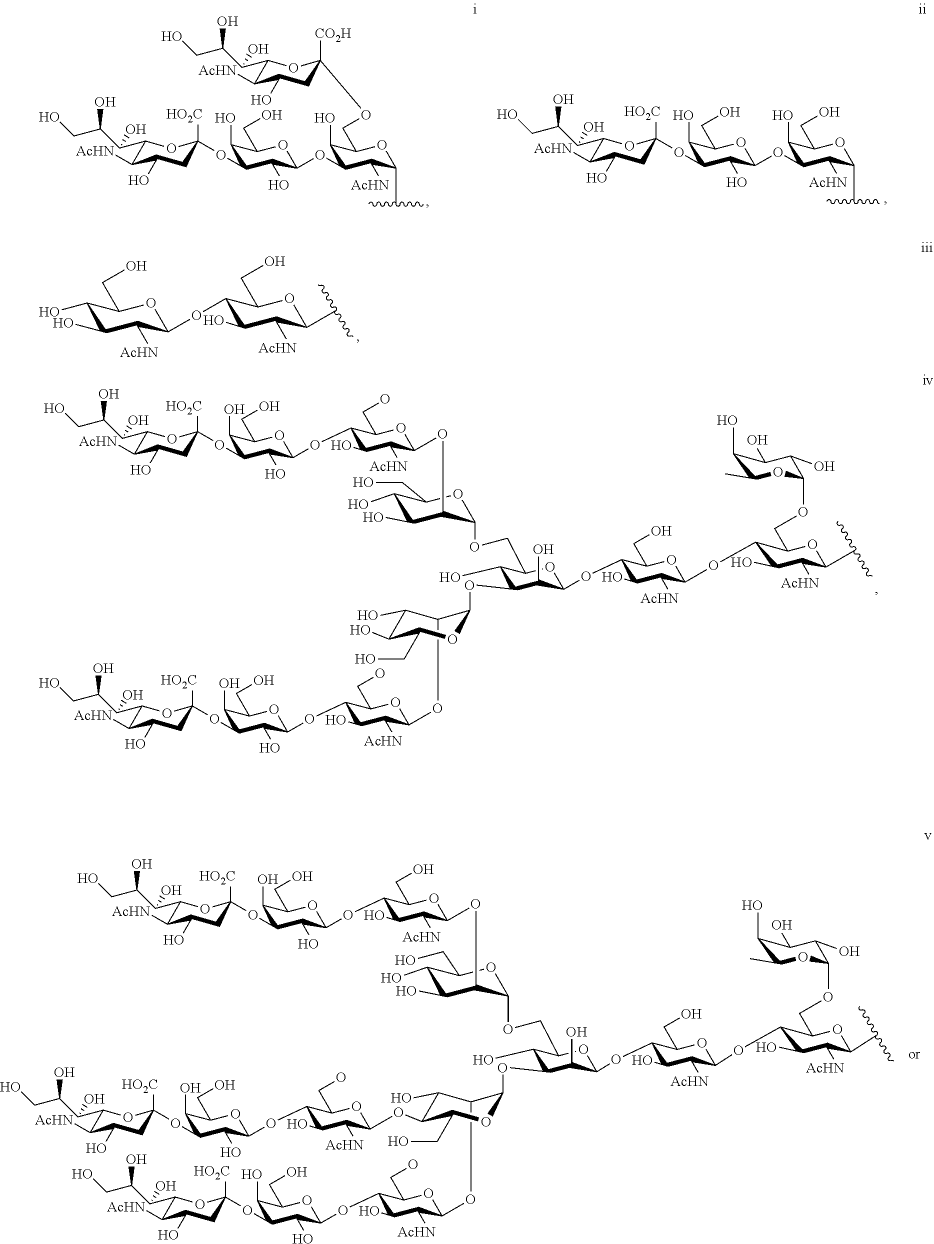

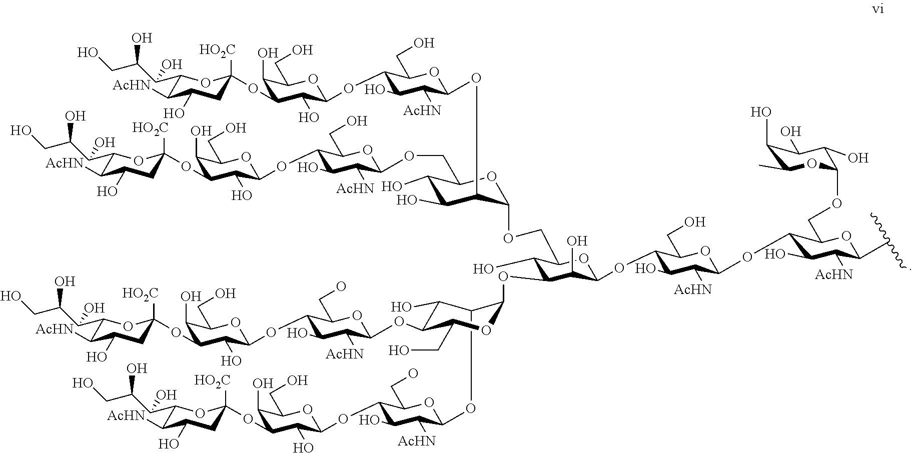

In some embodiments, a parathyroid hormone peptide and/or analog has an amino acid sequence which is ≧80%, ≧85%, ≧90% or ≧95% identical to SEQ ID NO: 1 or SEQ ID NO: 2 and is O-glycosylated. In some embodiments, a parathyroid hormone peptide and/or analog has an amino acid sequence which is ≧80%, ≧85%, ≧90% or ≧95% identical to SEQ ID NO: 1 or SEQ ID NO: 2 and is glycosylated at serine or threonine. In some embodiments, a parathyroid hormone peptide and/or analog has an amino acid sequence which is ≧80%, ≧85%, ≧90% or ≧95% identical to SEQ ID NO: 1 or SEQ ID NO: 2 and is glycosylated at S₁. In some embodiments, a parathyroid hormone peptide and/or analog has an amino acid sequence which is ≧80%, ≧85%, ≧90% or ≧95% identical to SEQ ID NO: 1 or SEQ ID NO: 2 and is glycosylated at S₁, wherein the glycan is selected from:

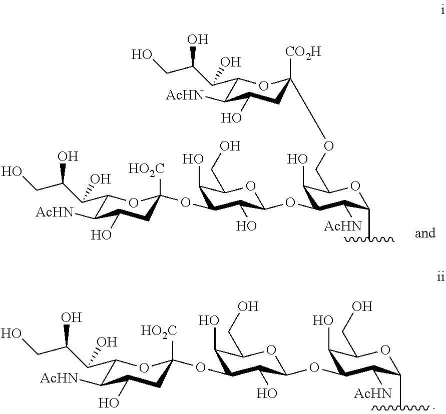

In some embodiments, a parathyroid hormone peptide and/or analog has an amino acid sequence which is ≧80%, ≧85%, ≧90% or ≧95% identical to SEQ ID NO: 1 or SEQ ID NO: 2 and is glycosylated at N₃₃, wherein the glycan is selected from

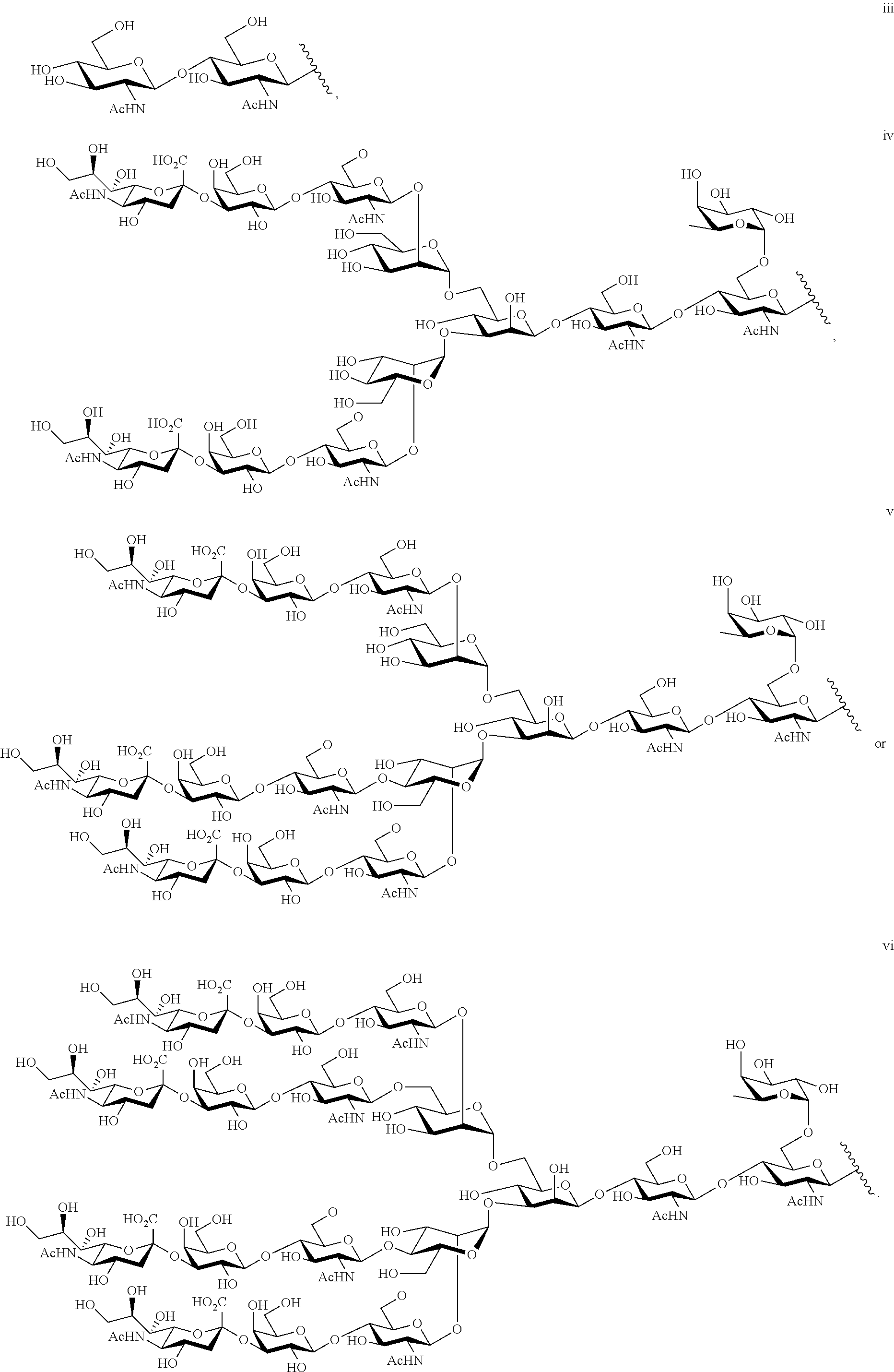

In some embodiments, a parathyroid hormone peptide and/or analog has an amino acid sequence which is ≧80%, ≧85%, ≧90% or ≧95% identical to SEQ ID NO: 1 or SEQ ID NO: 2 and is glycosylated at N₃₃, wherein the glycan is

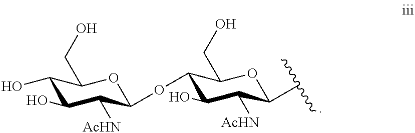

In other embodiments, a parathyroid hormone peptide and/or analog has an amino acid sequence which is ≧80%, ≧85%, ≧90% or ≧95% identical to SEQ ID NO: 1 or SEQ ID NO: 2 and is glycosylated at N₃₃, wherein the glycan is

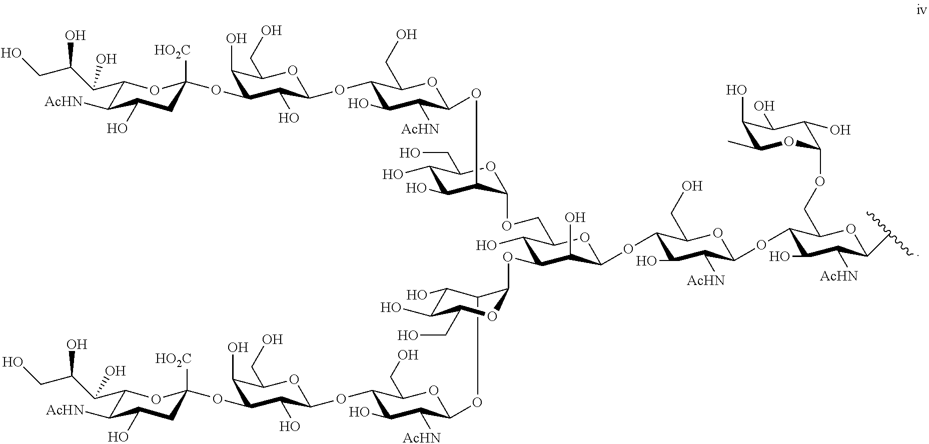

In certain embodiments, a parathyroid hormone peptide and/or analog has an amino acid sequence which is ≧80%, ≧85%, ≧90% or ≧95% identical to SEQ ID NO: 1 or SEQ ID NO: 2 and is glycosylated at N₃₃, wherein the glycan is

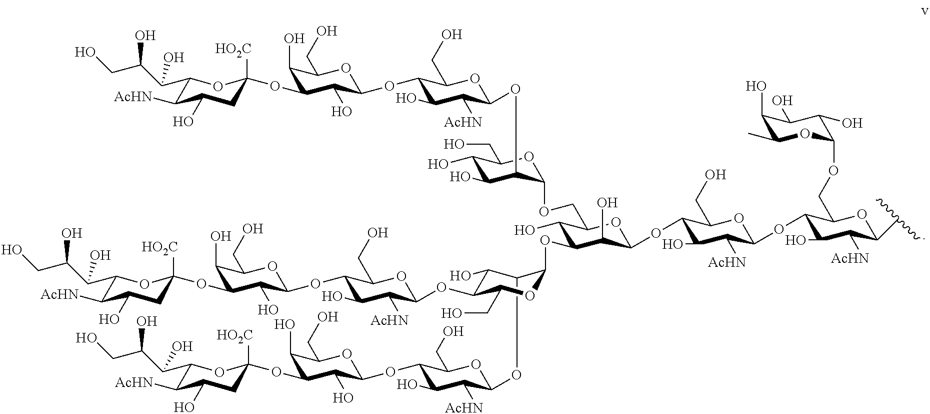

In some embodiments, a parathyroid hormone peptide and/or analog has an amino acid sequence which is ≧80%, ≧85%, ≧90% or ≧95% identical to SEQ ID NO: 1 or SEQ ID NO: 2 and is glycosylated at N₃₃, wherein the glycan is

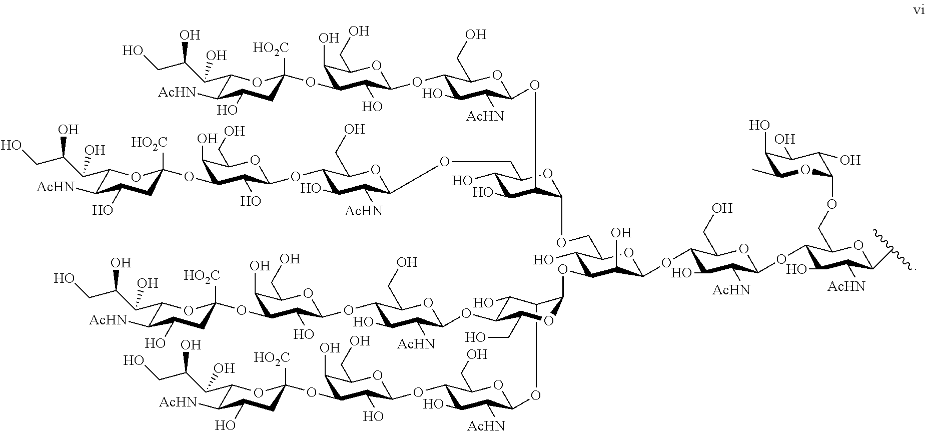

In some embodiments, the present invention provides a parathyroid hormone peptide and/or analog ≧80%, ≧85%, ≧90% or ≧95% identical to SEQ ID NO: 15, wherein the parathyroid hormone peptide and/or analog includes a norleucine and/or methoxinine residue at a position corresponding to residue 8, residue 18, and combinations thereof.

In some embodiments, the present invention provides a parathyroid hormone peptide and/or analog having an amino acid sequence which includes an element ≧80%, ≧85%, ≧90% or ≧95% identical to SEQ ID NO: 14, wherein the parathyroid hormone peptide and/or analog includes a norleucine and/or methoxinine residue at a position corresponding to residue 8, residue 18, and combinations thereof.

Parathyroid Hormone-Related Protein Peptides

The present invention also provides parathyroid hormone-related protein (PTHrP) peptides. Parathyroid hormone-related protein acts as an endocrine, autocrine, paracrine and intracrine hormone and regulates endochondral bone development by maintaining the endochondral growth plate at a constant width. hPTHrP further regulates epithelial-mesenchymal interactions during the formation of the mammary glands, and may regulate, in conjunction with the calcium sensing receptor, the mobilization and transfer of calcium to milk during lactation.

hPTHrP is widely expressed in normal and malignant tissues. It exists in three isoforms of 139, 141 and 173 amino acid-containing peptides. All three isoforms are synthesized from a common gene and differ only at the extreme carboxyl termini. The identification of the primary structure of hPTHrP in 1987 initiated the characterization of the structure-activity relationship of hPTHrP. Owing to the sequence similarity of the hPTHrP N-terminus to hPTH, hPTHrP can exert nearly identical functions that are mediated by the hPTH N-terminus. Accordingly, in some embodiments, the present invention provides analogs of hPTHrP. In some embodiments, the present invention provides stable hPTHrP therapeutics. In some embodiments, hPTHrP analogs have greater stability than wild type hPTHrP and/or its isoforms (e.g., when measured in an in vitro peptide stability assay in human serum).

hPTHrP shares little sequence homology with the C-terminal domain of hPTH. These sequence differences enable the distinct functions of hPTHrP in normal and cancer tissues.

The sequence of human hPTHrP is shown in SEQ ID NO: 8. In some embodiments, the present invention provides a parathyroid hormone-related protein peptide and/or analog. In certain embodiments, the present invention provides a hPTHrP peptide and/or analog 3-180 amino acids in length. In some embodiments, provided hPTHrP peptides and/or analogs have an amino acid sequence that is at least a minimum length and not more than a maximum length, wherein the minimum length is, for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more amino acids, and where the maximum length is not more than 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 149, 148, 147, 146, 145, 144, 143, 142, 141, 140, 139, 138, 137, 136, 135, 134, 133, 132, 131 or 130 amino acids in length.

In certain embodiments, the present invention provides one or more isoforms of hPTHrP. In some embodiments, the present invention provides a hPTHrP peptide and/or analog 139-amino acids in length.

In some embodiments, the present invention provides a hPTHrP peptide and/or analog 141-amino acids in length.

In some embodiments, the present invention provides a hPTHrP peptide and/or analog 173-amino acids in length.

SEQ ID NO: 8 depicts one wild-type isoform of hPTHrP. In some embodiments, a provided hPTHrP peptide and/or analog has an amino acid sequence ≧80%, ≧85%, ≧90% or ≧95% identical to SEQ ID NO: 8. In some embodiments, a hPTHrP peptide and/or analog has an amino acid sequence ≧80%, ≧85%, ≧90% or ≧95% identical to SEQ ID NO: 9. In some embodiments, a provided hPTHrP peptide and/or analog has an amino acid sequence ≧80%, ≧85%, ≧90% or ≧95% identical to SEQ ID NO: 16. In some embodiments, a provided hPTHrP peptide and/or analog has an amino acid sequence ≧80%, ≧85%, ≧90% or ≧95% identical to SEQ ID NO: 17. In some embodiments, provided hPTHrP peptide and/or analog has an amino acid sequence that is overall ≧80%, ≧81%, ≧82%, ≧83%, ≧84%, ≧85%, ≧86%, ≧87%, ≧88%, ≧89%, ≧90%, ≧91%, ≧92%, ≧93%, ≧94%, ≧95%, ≧96%, ≧97%, ≧98%, ≧99% or more identical to SEQ ID NOs: 8, 9, 16 or 17.

SEQ ID NOs: 10, 11, 12 and 13 depict conserved regions of hPTHrP across various species. Accordingly, in some embodiments, a provided hPTHrP peptide and/or analog has an amino acid sequence which includes at least one of SEQ ID NO: 10, SEQ ID NO: 11, SEQ ID NO: 12 and SEQ ID NO: 13.

In some embodiments, the present invention provides a hPTHrP peptide and/or analog glycosylated with at least one glycan group. In some embodiments, the at least one glycan group is selected from:

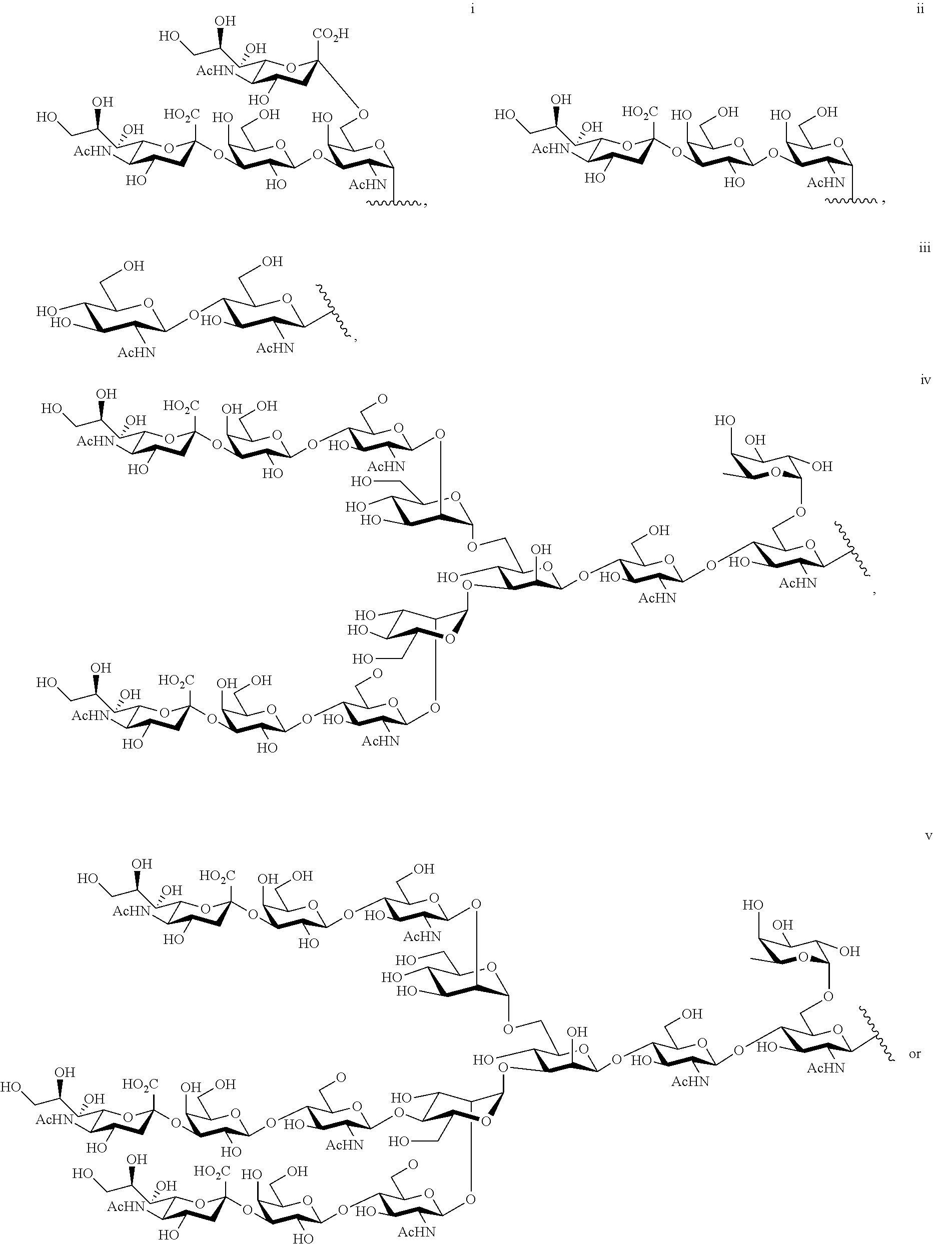

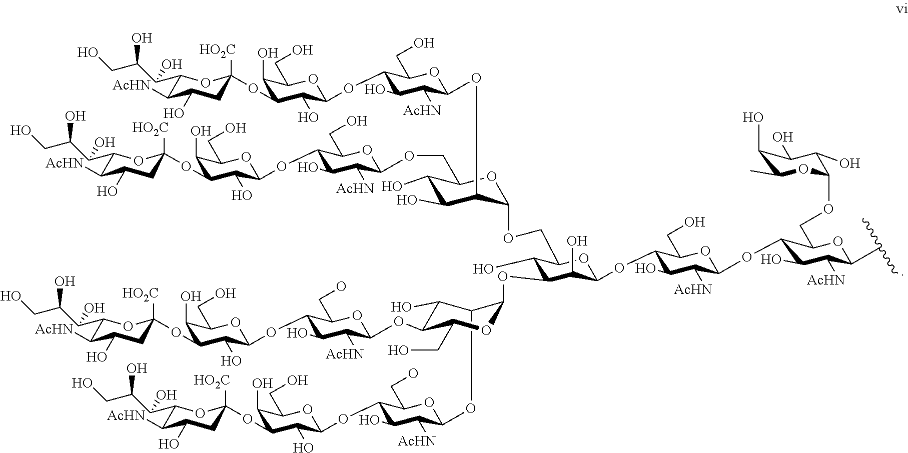

Particular Parathyroid Hormone-Related Protein Analogs

In some embodiments, a hPTHrP peptide and/or analog has an amino acid sequence ≧80%, ≧85%, ≧90% or ≧95% identical to SEQ ID NO: 8 and includes at least one of SEQ ID NO: 10, SEQ ID NO: 11, SEQ ID NO: 12 and SEQ ID NO: 13.

In some embodiments, a hPTHrP peptide and/or analog has an amino acid sequence ≧80%, ≧85%, ≧90% or ≧95% identical to SEQ ID NO: 9 and includes at least one of SEQ ID NO: 10, SEQ ID NO: 11, SEQ ID NO: 12 and SEQ ID NO: 13.

Peptide Synthesis

The availability of the hPTH and hPTHrP and their fragments in pure form is a prerequisite for studying the biological functions of hPTH or hPTHrP. Because hPTHrP contains no cysteine residues, the chemical synthesis of hPTHrP via native chemical ligation has been problematic. In general biological methods and/or chemical methods can be used for the production of provided hPTH and/or hPTHrP polypeptides as described herein. However, those of ordinary skill in the art will appreciate that biological methods (for example such as recombinant DNA-based methods) may not be suitable for incorporating unnatural amino acids, and particularly for incorporating multiple unnatural amino acids. (Voloshchuk N, Montclare J K. 2010. “Incorporation of unnatural amino acids for synthetic biology.” Mol. Biosyst. 6: 65-80).

Utilizing chemical synthesis in the production of peptides and/or proteins offers the potential of solving a multitude of problems in biomedical sciences. Chemical synthesis can exert great control on the protein composition. Moreover, chemical synthesis can facilitate the creation of new proteins with desirable properties. Historically, the chemical preparation of biotherapeutic proteins and their analogs has relied on the use of the powerful cysteine-based native chemical ligation (NCL) method of Kent and associates. (Dawson P E, Muir T W, Clark-Lewis I, Kent S B (1994) Synthesis of proteins by native chemical ligation. Science 266:776-779; Tam J P, Lu Y A, Liu C F, Shao J (1995) Peptide synthesis using unprotected peptides through orthogonal coupling methods. Proc Natl Acad Sci USA 92:12485-12489; Hua Q X, Nakagawa S H, Jia W, Huang K, Phillips N B, Hu S Q, Weiss M A. 2008). “Design of an active ultrastable single-chain insulin analog: synthesis, structure, and therapeutic implications.” J. Biol. Chem. 283: 14703-16). However, given the relative scarcity of cysteine residues in nature, existing NCL methodologies are often not useful or effective for the production of certain peptides or proteins. hPTH is one of many proteins which lacks cysteine residues, thus rendering NCL impractical for the efficient generation of chemical analogs of hPTH. (Dawson P E, Muir T W, Clark-Lewis I, Kent S B. 1994. “Synthesis of proteins by native chemical ligation.” Science 266: 776-9).

Previously, the chemical synthesis of hPTH required either the solid phase synthesis of 84-mer-long peptide or the assembly of fully protected peptide segments, which are tedious and impractical for the generation of analogs. (Kimura T, Takai M, Masui Y, Morikawa T, Sakakibara S. 1981. “Strategy for the Synthesis of Large Peptides—an Application to the Total Synthesis of Human Parathyroid-Hormone [hPTH(1-84)].” Biopolymers 20: 1823-32; Fairwell T, Hospattankar A V, Ronan R, Brewer H B, Jr., Chang J K, Shimizu M, Zitzner L, Arnaud C D. 1983. “Total solid-phase synthesis, purification, and characterization of human parathyroid hormone-(1-84).” Biochemistry 22: 2691-7; Goud N A, McKee R L, Sardana M K, DeHaven P A, Huelar E, Syed M A/I, Goud R A, Gibbons S W, Fisher J E, Levy J J, et al. 1991. “Solid-phase synthesis and biologic activity of human parathyroid hormone (1-84).” J. Bone Miner. Res. 6: 781-9; Fuentes G, Page K, Chantell C A, Patel H, Menakuru M. 2009. “Fast conventional synthesis of human parathyroid hormone 1-84.” Chim. Oggi 27: 31-3).

In order to make the chemical synthesis of hPTH and its analogs more attractive than by other methods, researchers have considerably extended the applicability of the native chemical ligation method. (Wan Q, Danishefsky S J. 2007. “Free-radical-based, specific desulfurization of cysteine: a powerful advance in the synthesis of polypeptides and glycopolypeptides.” Angew. Chem. Int. Ed. 46: 9248-52; Chen J, Wan Q, Yuan Y, Zhu J, Danishefsky S J. 2008. “Native chemical ligation at valine: a contribution to peptide and glycopeptide synthesis.” Angew. Chem. Int. Ed. 47: 8521-4; Chen J, Wang P, Zhu J L, Wan Q, Danishefsky S J. 2010. “A program for ligation at threonine sites: application to the controlled total synthesis of glycopeptides.” Tetrahedron 66: 2277-83; Tan Z, Shang S, Danishefsky S J. 2010. “Insights into the Finer Issues of Native Chemical Ligation: An Approach to Cascade Ligations.” Angew. Chem. Int. Ed., 49: 9500-9503). Using a coupled non-cysteine-based ligation/desulfurization strategies, the full-length hPTH molecule can be assembled from small synthetic peptide fragments, which would in turn enable flexible modification of its natural structure. (Tam J P, Yu Q T. 1998. “Methionine ligation strategy in the biomimetic synthesis of parathyroid hormones.” Biopolymers 46: 319-27).

In certain embodiments, the present invention provides methods of synthesizing parathyroid hormone, parathyroid hormone-related protein and/or peptides and/or analogs thereof. In certain embodiments, the present invention provides methods of synthesizing hPTH, hPTHrP and peptides and/or analogs thereof, comprising at least one native chemical ligation coupling at an amino acid residue other than cysteine or methionine. In some embodiments, the present invention provides methods of synthesizing hPTH, hPTHrP and/or peptides and/or analogs thereof, comprising at least one native chemical ligation coupling at an amino acid residue selected from alanine, valine, threonine, leucine and proline. In some embodiments, the present invention provides a method of synthesizing hPTH of SEQ ID NO: 1:

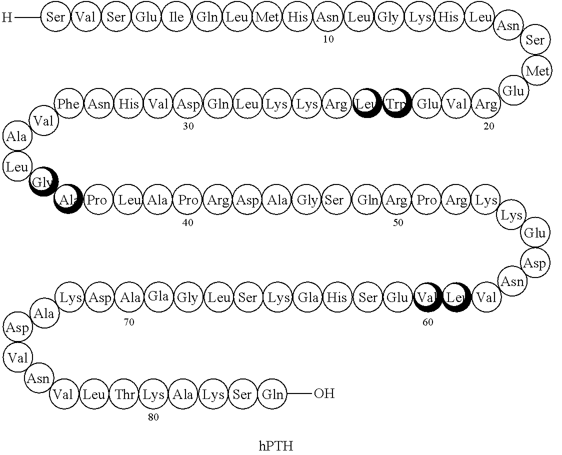

In some embodiments, the present invention provides a synthesis of hPTH comprising the native chemical ligation of fragments I, II, III and IV:

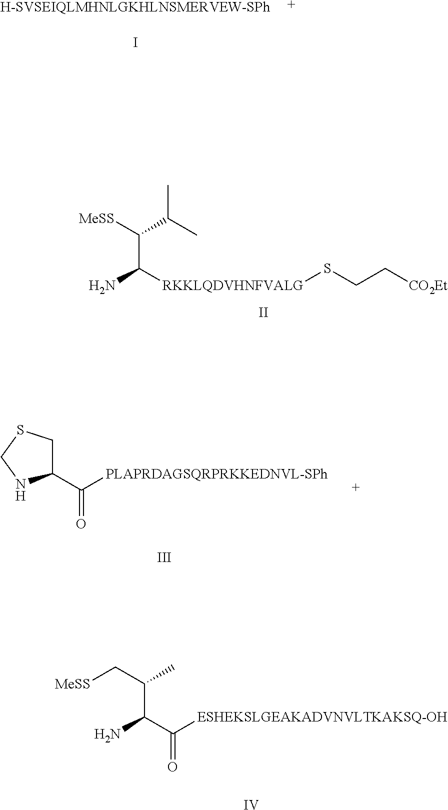

In some embodiments, the present invention provides a synthesis of hPTH comprising the native chemical ligation of fragments I and II to produce fragment V:

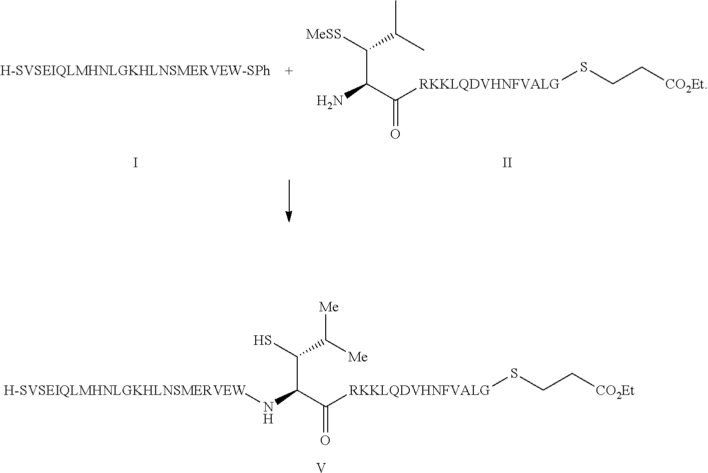

In some embodiments, the present invention provides a synthesis of hPTH comprising the native chemical ligation of fragments III and IV to produce fragment VI:

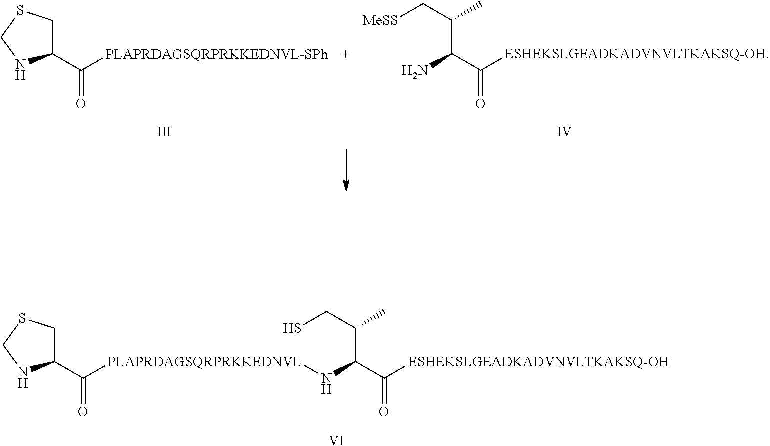

In some embodiments, the present invention provides a synthesis of hPTH comprising the native chemical ligation of fragments III and IV to produce fragment VI, followed by the deprotection of the N-terminus to produce fragment VII:

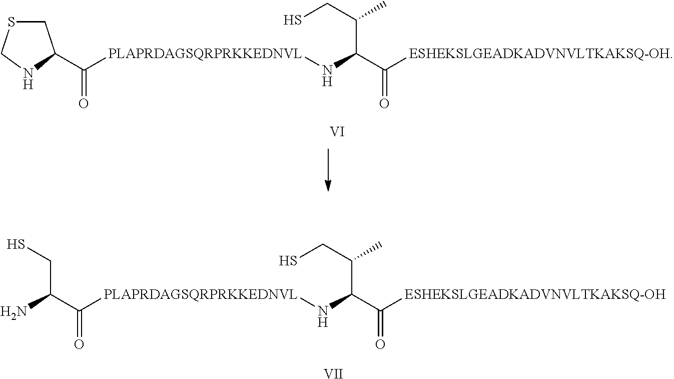

In some embodiments, the present invention provides a synthesis of hPTH comprising the native chemical ligation of fragments V and VII:

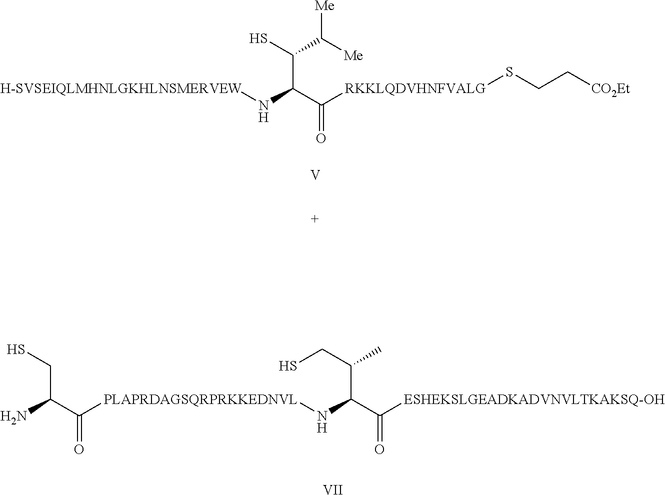

In some embodiments, the present invention provides a synthesis of hPTH comprising the native chemical ligation of fragments V and VII, followed by the desulfurization of fragment VIII to yield hPTH (1-84).

In some embodiments, the present invention provides a method of preparing a hPTH peptide comprising:

(i) native chemical ligation of fragments I and II to produce fragment V:

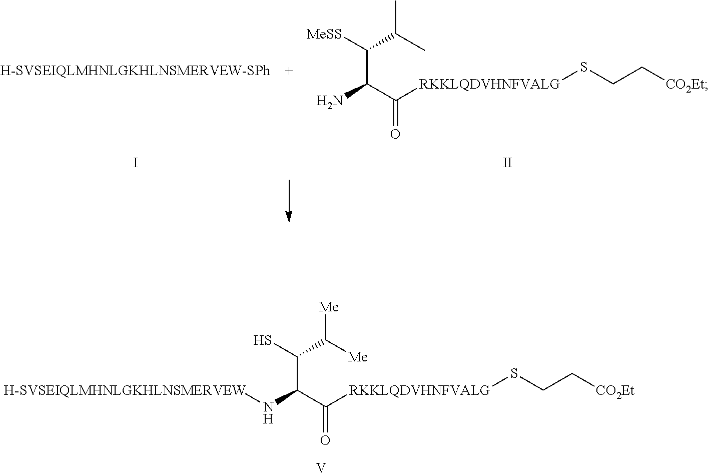

(ii) native chemical ligation of fragments III and IV to produce fragment VI:

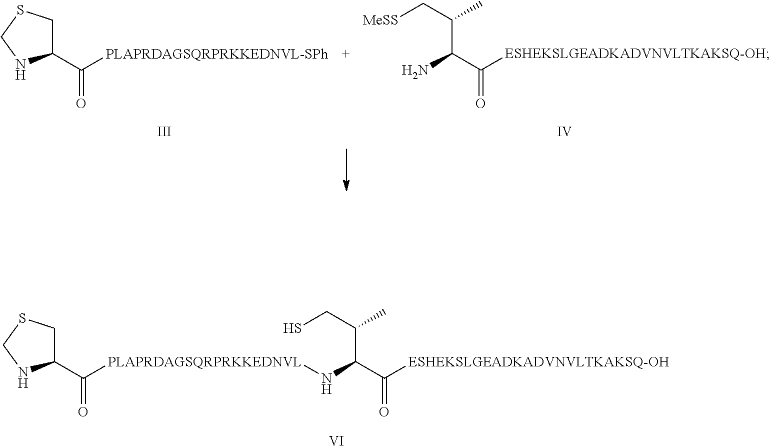

(iii) deprotecting fragment VI to produce fragment VII:

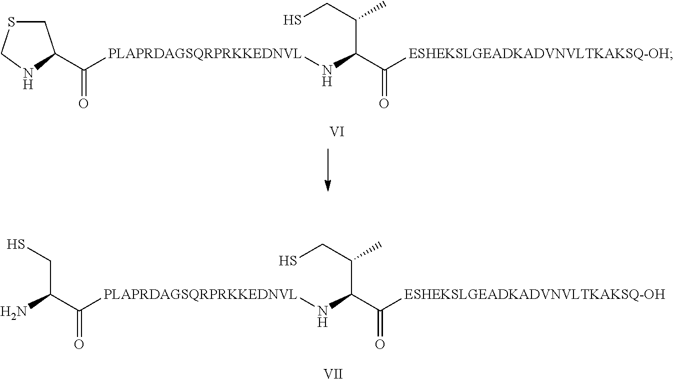

(iv) native chemical ligation of fragments V and VII to produce fragment VIII:

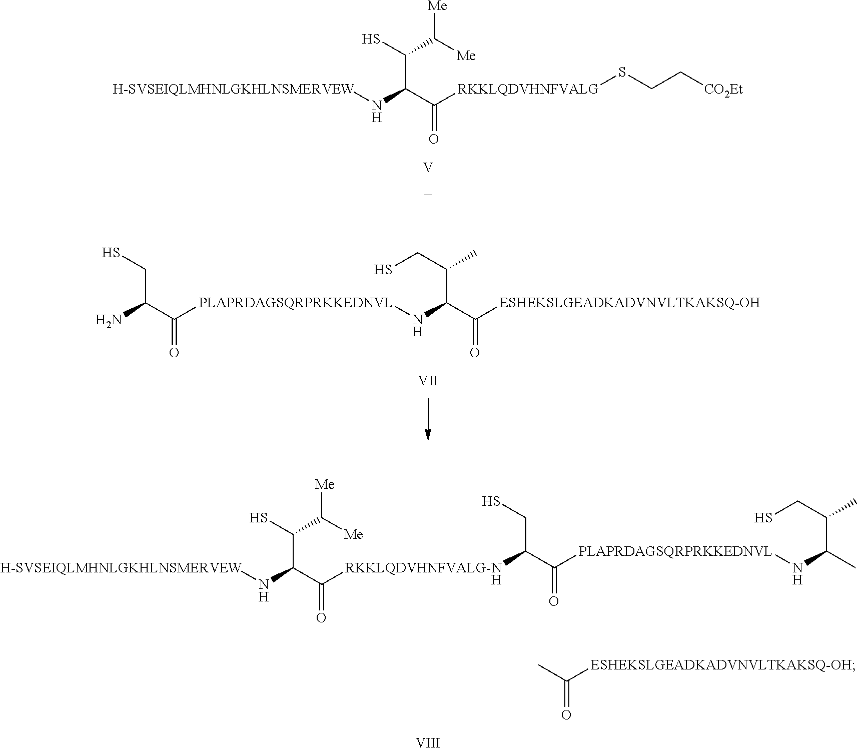

and (v) reduction of fragment VIII to produce an hPTH peptide:

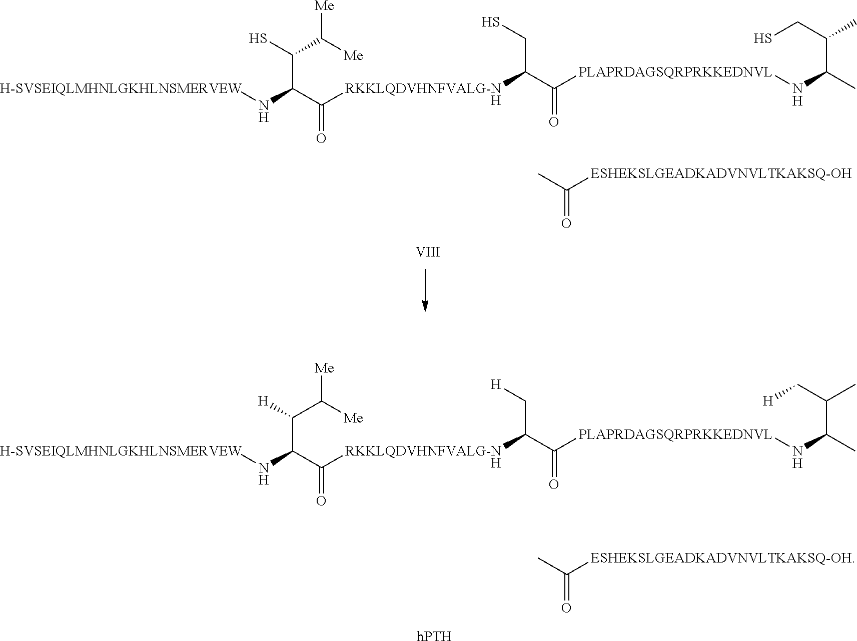

In some embodiments, the present invention provides a method of synthesizing a hPTH analog A of SEQ ID NO: 14 wherein the sequence includes a norleucine at positions corresponding to residues 8 and 18:

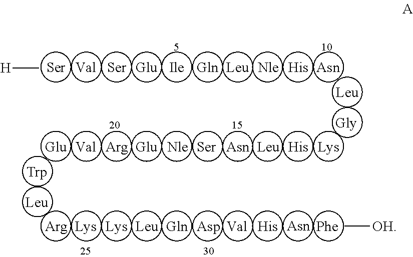

In some embodiments, the present invention provides a synthesis of hPTH analog A comprising the native chemical ligation of fragments IX and XVII:

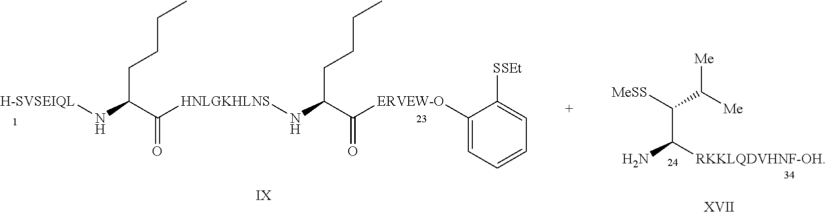

In some embodiments, the present invention provides a synthesis of hPTH analog A comprising the native chemical ligation of fragments XVIII and XIX:

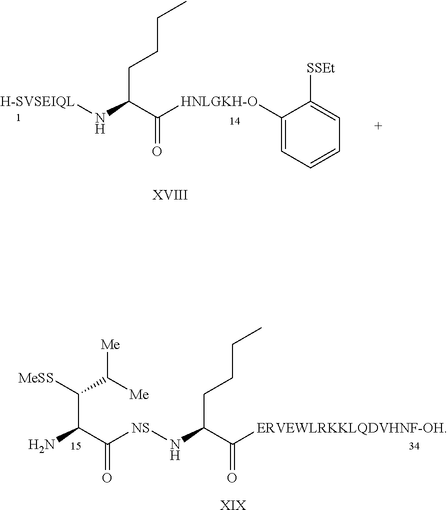

In some embodiments, the present invention provides a method of synthesizing a hPTH analog of SEQ ID NO: 14 wherein the peptide is glycosylated with at least one glycan group. In some embodiments, the present invention provides a method of synthesizing a hPTH analog of SEQ ID NO: 14 wherein the peptide is glycosylated with at least one glycan group and wherein the sequence includes a norleucine at positions corresponding to residues 8 and 18. In some embodiments, the present invention provides a method of synthesizing a glycosylated hPTH analog B:

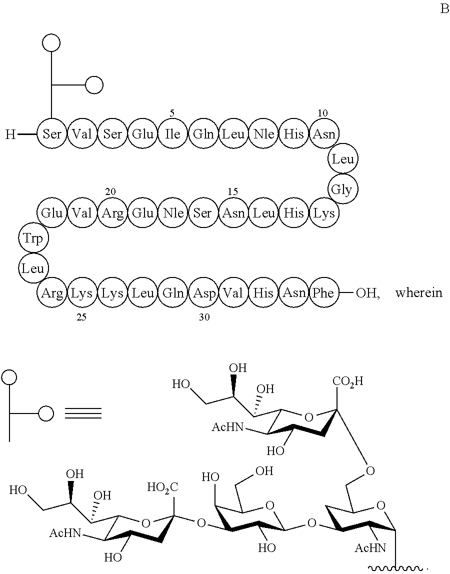

In some embodiments, the present invention provides a synthesis of hPTH analog B comprising the native chemical ligation of fragments XX, XXI and XXII:

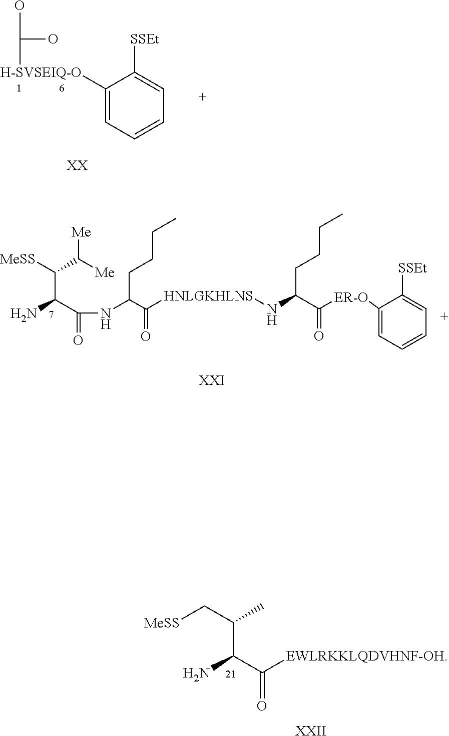

In some embodiments, the present invention provides a method of synthesizing a glycosylated hPTH analog C:

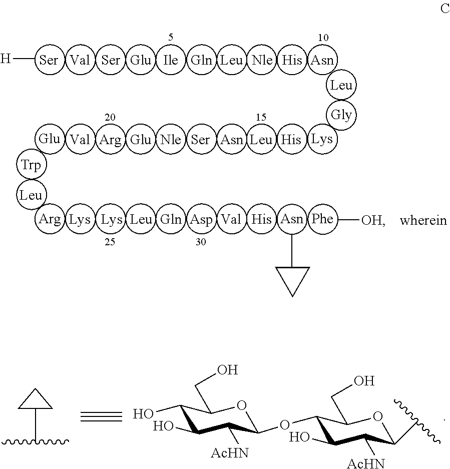

In some embodiments, the present invention provides a synthesis of hPTH analog C comprising the native chemical ligation of fragments XVIII, XXIII and XXIV:

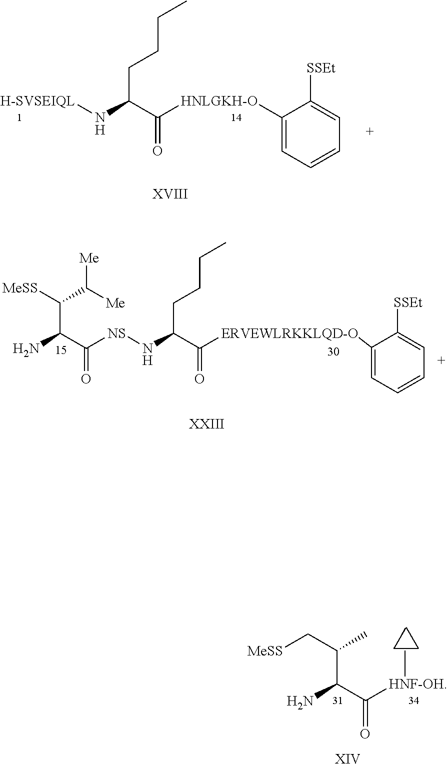

In some embodiments, the present invention provides a method of synthesizing a glycosylated hPTH analog D:

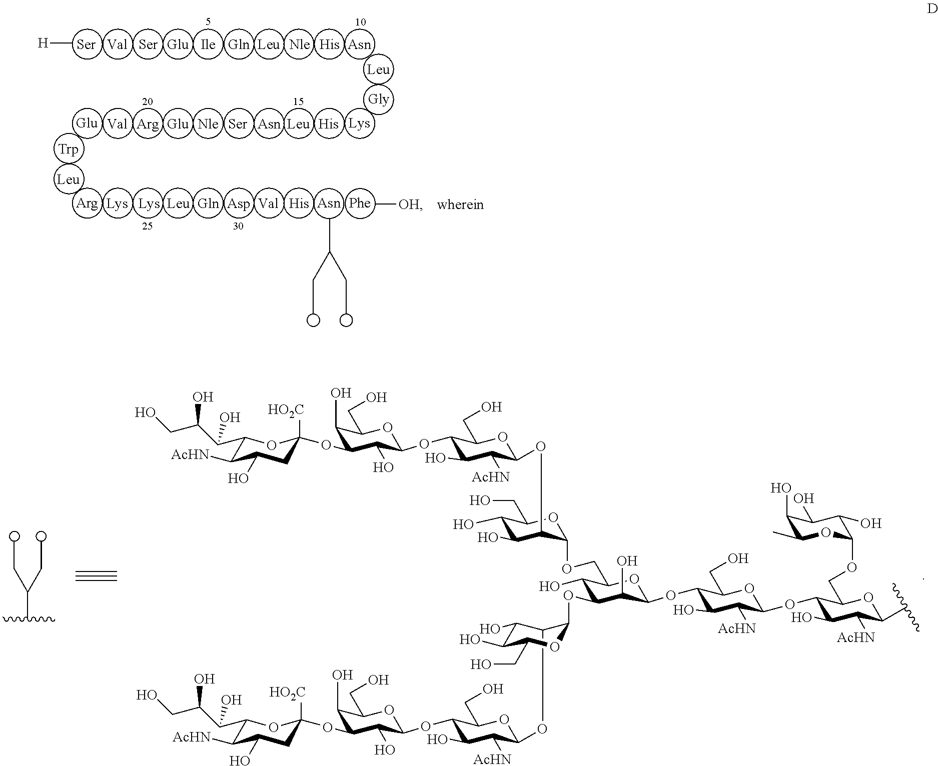

In some embodiments, the present invention provides a synthesis of hPTH analog D comprising the native chemical ligation of fragments XVIII, XXIII and XXV:

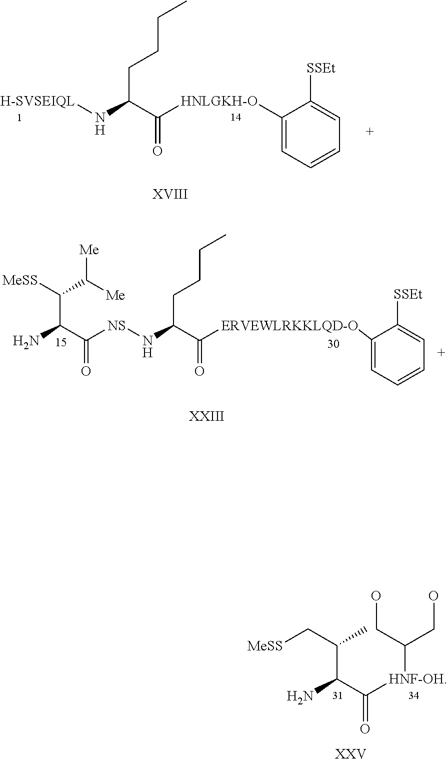

In some embodiments, the present invention provides a method of synthesizing a hPTH analog E of SEQ ID NO: 1, wherein the sequence includes a norleucine at positions corresponding to residues 8 and 18:

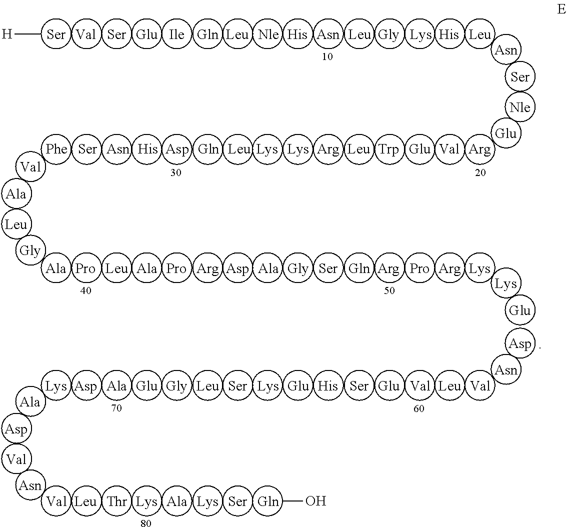

In some embodiments, the present invention provides a synthesis of hPTH analog E comprising the native chemical ligation of fragments IX, II and X:

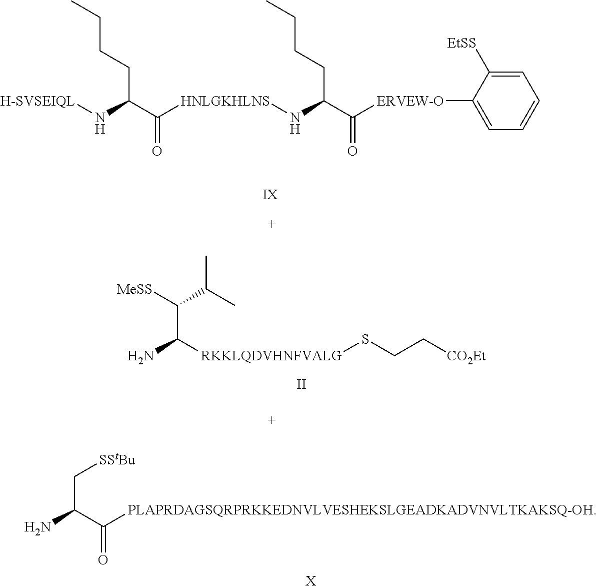

In some embodiments, the present invention provides a method of synthesizing a glycosylated analog F of SEQ ID NO: 1, wherein the sequence includes a norleucine at positions corresponding to residues 8 and 18:

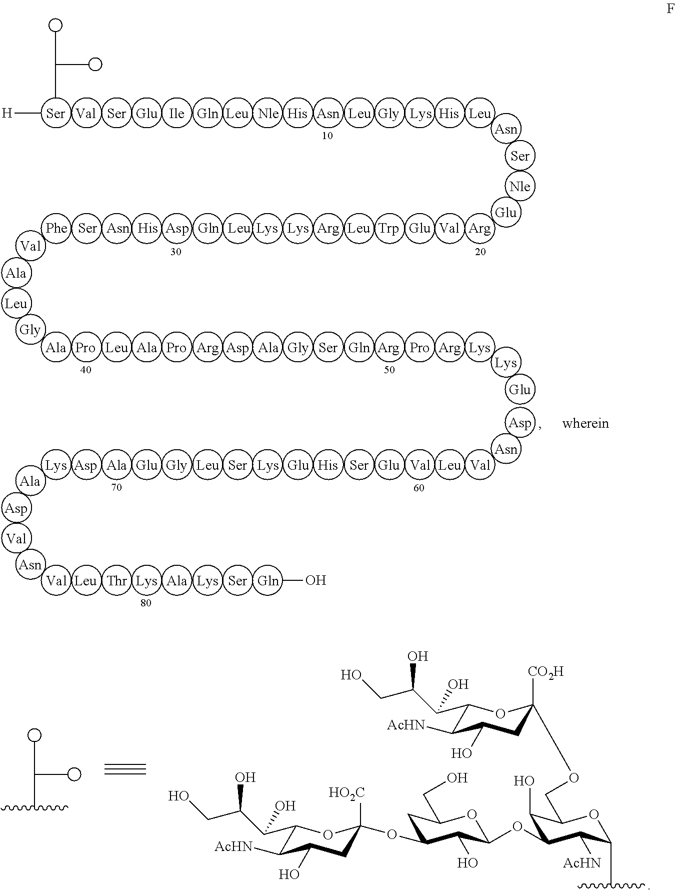

In some embodiments, the present invention provides a synthesis of hPTH analog F comprising the native chemical ligation of fragments XX, XXVI and II and X:

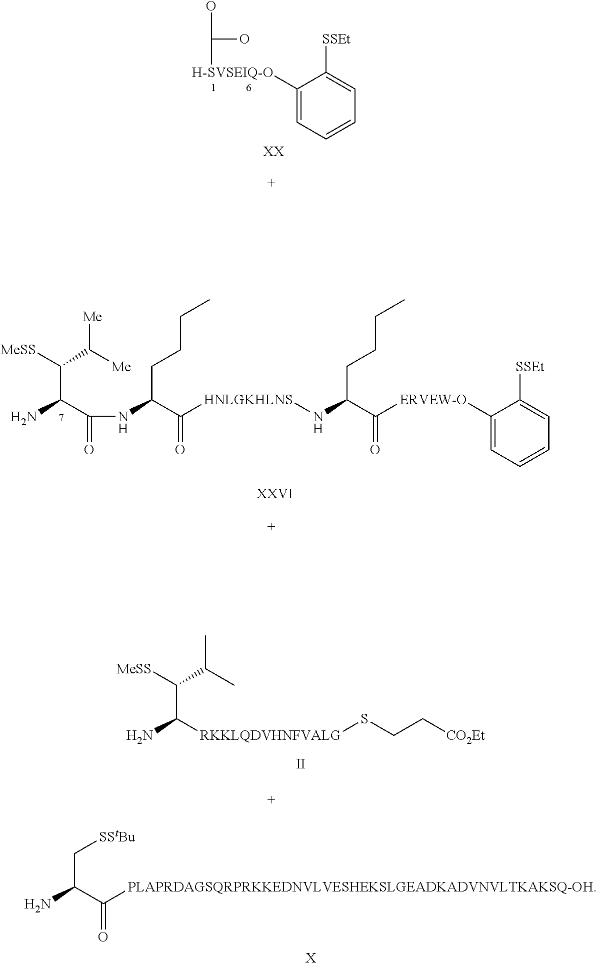

In some embodiments, the present invention provides a method of synthesizing a glycosylated analog G of SEQ ID NO: 1, wherein the sequence includes a norleucine at positions corresponding to residues 8 and 18:

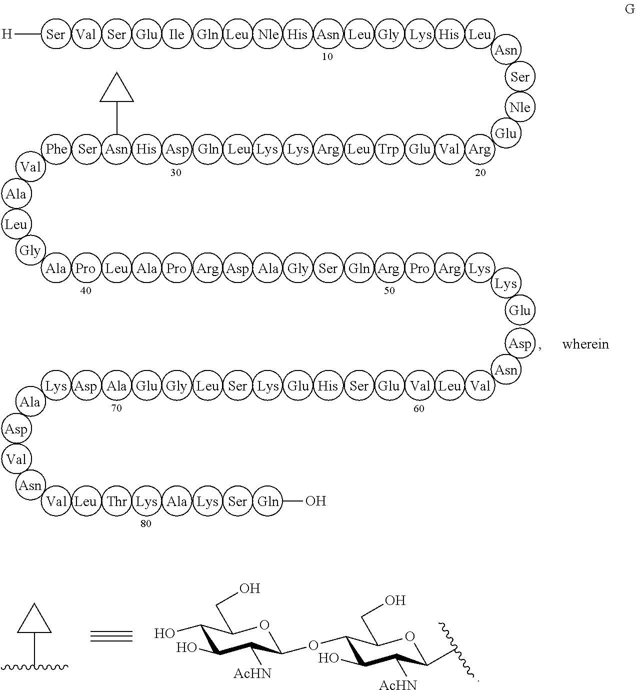

In some embodiments, the present invention provides a synthesis of hPTH analog G comprising the native chemical ligation of fragments XXVII, XXVIII and X:

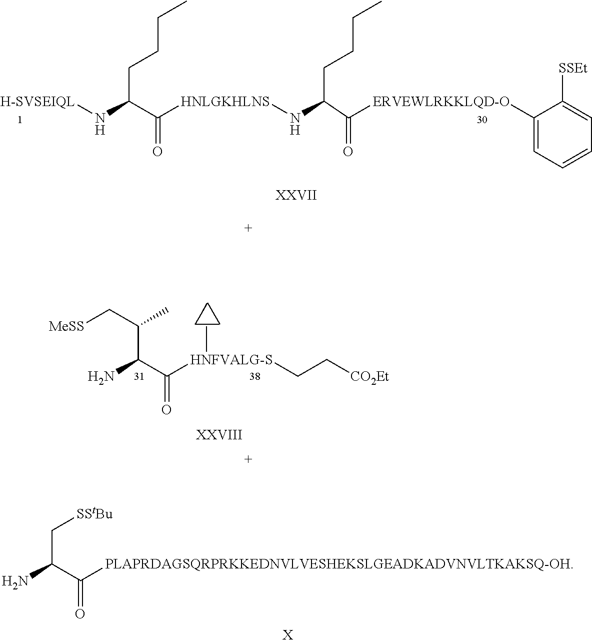

In some embodiments, the present invention provides a method of synthesizing a glycosylated analog H of SEQ ID NO: 1, wherein the sequence includes a norleucine at positions corresponding to residues 8 and 18:

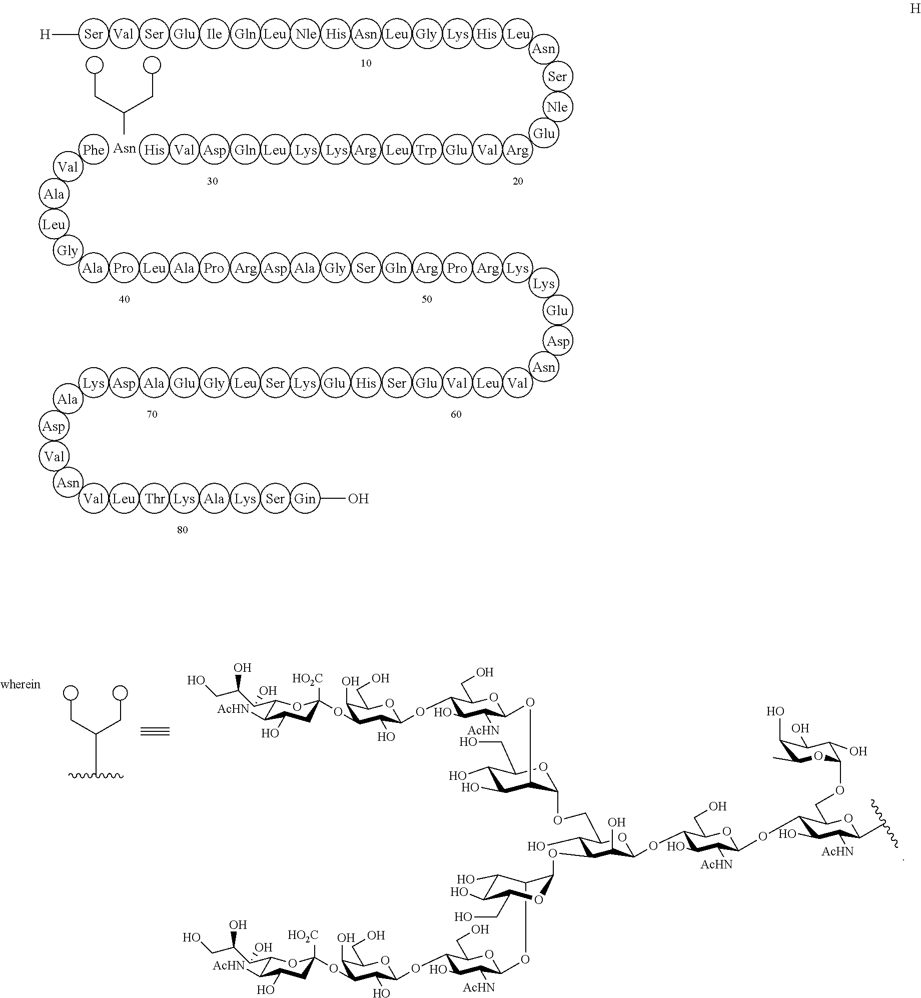

In some embodiments, the present invention provides a synthesis of hPTH analog H comprising the native chemical ligation of fragments XXVII, XXIX and X:

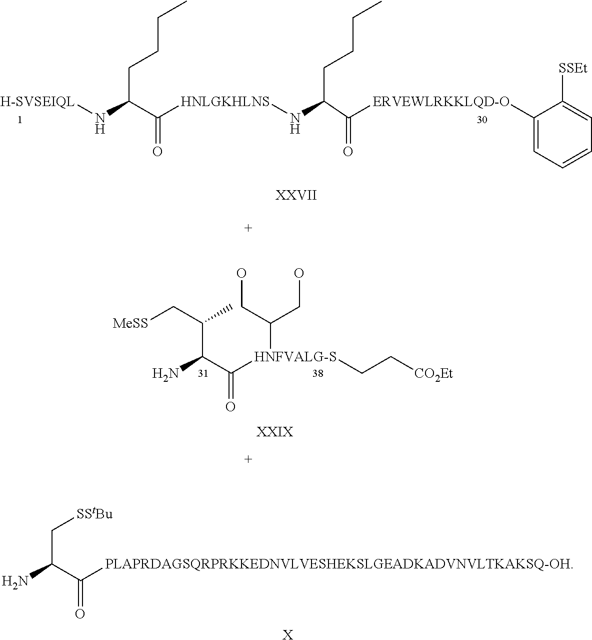

Human parathyroid hormone-related protein contains no cysteine or methionine residues, and consequently cannot be synthesized by conventional native chemical ligation methods. In some embodiments, the present invention provides a method of synthesizing a hPTHrP peptide of SEQ ID NO: 8 comprising the native chemical ligation of fragments of XXX, XXXI, XXXII and XXXIII.

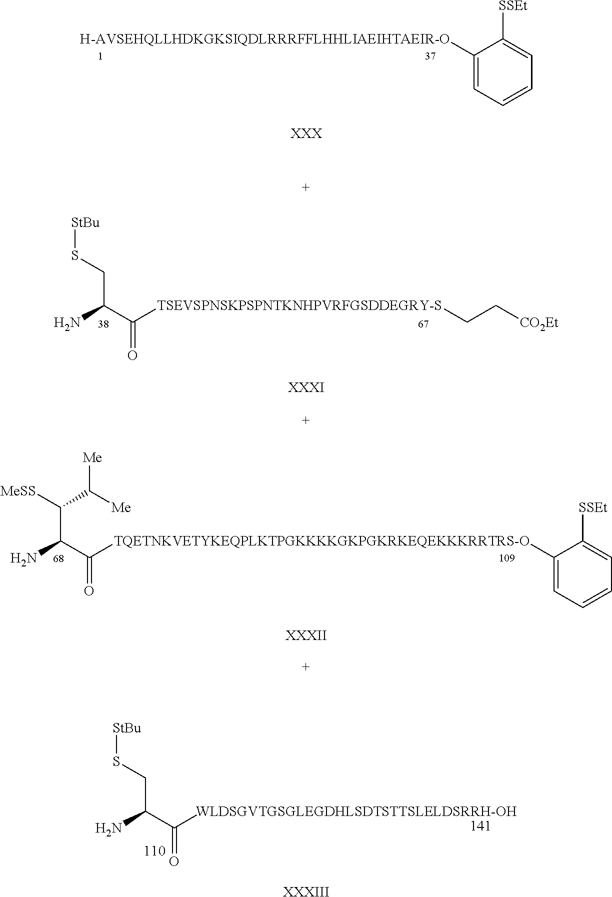

In some embodiments, the present invention provides the synthesis of intermediate XXXIV:

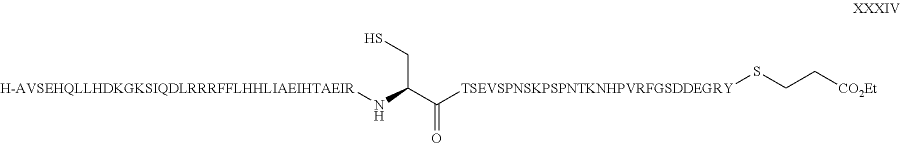

comprising the native chemical ligation of intermediates XXX and XXXI:

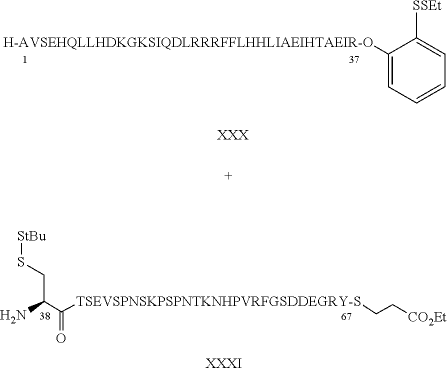

In some embodiments, the present invention provides the synthesis of intermediate XXXV:

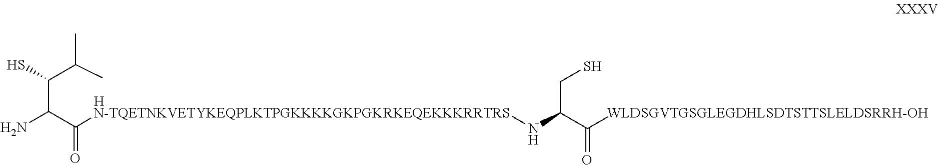

comprising the native chemical ligation of intermediates XXXII and XXXIII:

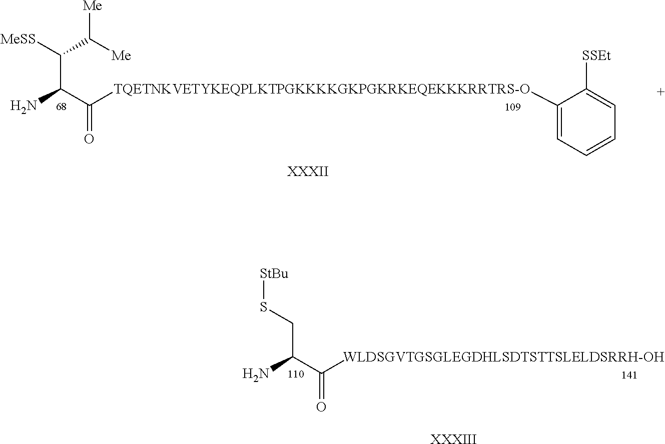

In some embodiments, the present invention provides the synthesis of intermediate XXXVI:

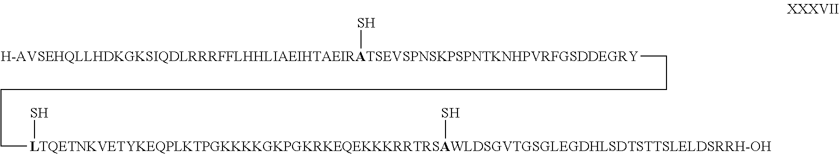

comprising the native chemical ligation of intermediates XXXIV and XXXV.

In some embodiments, the present invention provides the synthesis of hPTHrP XXXVII:

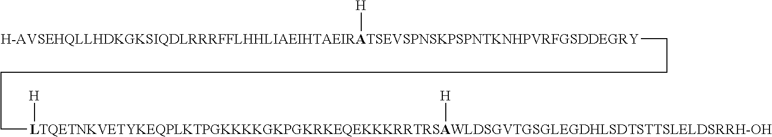

comprising reducing intermediate XXXVI with a desulfurization agent.

In certain embodiments, the present invention provides native chemical ligation intermediates. In some embodiments, the present invention provides native chemical ligation intermediates I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV and XXXVI.

Uses of Compounds and Pharmaceutically Acceptable Compositions

According to some embodiments, the invention provides a composition comprising a peptide and/or analog of this invention, optionally in the form of a pharmaceutically acceptable salt, ester, or other derivative thereof, and a pharmaceutically acceptable carrier, adjuvant, or vehicle.

In some embodiments, a pharmaceutically acceptable composition comprises and/or provides upon administration a therapeutically effective amount of a hPTH or hPTHrP peptide and/or analog. In some embodiments, a pharmaceutically acceptable composition comprises and/or provides upon administration a therapeutically effective amount of a hPTH or hPTHrP peptide and/or analog.

In some embodiments, the present invention provides a pharmaceutical composition comprising a hPTH peptide and/or analog and at least one pharmaceutically acceptable carrier. In certain embodiments, the present invention provides a pharmaceutical composition comprising a hPTH peptide and/or analog and at least one pharmaceutically acceptable carrier, wherein the composition further comprises an additional therapeutic agent.

In some embodiments, the present invention provides a pharmaceutical composition comprising a hPTHrP peptide and/or analog and at least one pharmaceutically acceptable carrier. In certain embodiments, the present invention provides a pharmaceutical composition comprising a hPTHrP peptide and/or analog and at least one pharmaceutically acceptable carrier, wherein the composition further comprises an additional therapeutic agent.

In certain embodiments, a composition of this invention is formulated for administration to a patient in need of such composition. In some embodiments, a composition of this invention is formulated for oral administration to a patient.

Compositions of the present invention are useful in the treatment of symptoms, diseases and/or disorders associated with insufficient levels of parathyroid hormone. In some embodiments, compositions of the present invention are useful in the treatment of symptoms, diseases and/or disorders associated with hypoparathyroidism. In some embodiments, compositions of the present invention are useful in the treatment of symptoms, diseases and/or disorders associated with underactive parathyroid hormone. In some embodiments, compositions of the present invention are useful in the treatment of osteoporosis.

Compositions of the present invention may be administered by any appropriate route, for example orally, parenterally, by inhalation spray, topically, rectally, nasally, buccally, vaginally or via an implanted reservoir.

For parenteral administration, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. In some embodiments, provided peptides and/or analogs are administered parenterally.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex/gender, diet, time of administration, rate of excretion, drug combination, and the judgment of the treating physician and the severity of the particular disease being treated. The amount of a compound of the present invention in the composition will also depend upon the particular compound in the composition.

Teriparatide, marketed as FORTEO®, is a hPTH peptide 34-amino acids in length is currently approved by the Federal Drug Administration (FDA) for the treatment of postmenopausal women with osteoporosis at high risk for fracture. Teriparatide is also approved for the treatment of both men and women with osteoporosis associated with sustained systemic glucocorticoid therapy at high risk for fracture. Teriparatide further increases bone mass in men with primary or hypogonadal osteoporosis at high risk for fracture.

In some embodiments of the present invention, hPTH or hPTHrP peptides and/or analogs of the present invention have an activity as described herein. In some embodiments, hPTH or hPTHrP peptides and/or analogs promote restoration of serum calcium levels. Thus, in certain embodiments, the present invention provides a method for treating a disease and/or disorder characterized by insufficient parathyroid levels comprising the step of administering to a subject in need thereof a compound of the present invention, or pharmaceutically acceptable composition thereof.

In some embodiments, the present invention provides a method of treating a symptom, disease or disorder associated with insufficient levels of hPTH or hPTHrP. In some such embodiments, the present invention provides methods of treating hypothyroidism comprising administering to a subject in need thereof a therapeutically effective amount of a hPTH or hPTHrP peptide and/or analog. In some embodiments, the present invention provides a method for treating or lessening the severity of osteoporosis. In some embodiments, the present invention provides a method for treating or lessening the severity of osteoporosis comprising administering to a subject in need thereof a hPTH or hPTHrP peptide and/or analog. In some embodiments, the present invention provides a method for treating or lessening the severity of osteoporosis in postmenopausal women.

In some embodiments, the present invention provides a method for treating or lessening the severity of osteoporosis comprising administering to a subject in need thereof a hPTH or hPTHrP peptide and/or analog in combination with calcium and/or vitamin D.

In some embodiments, the present invention provides a method for increasing bone mineral density comprising administering to a subject in need thereof a hPTH or hPTHrP peptide and/or analog. In some embodiments, the present invention provides a method for increasing bone mineral density comprising administering to a subject in need thereof a hPTH or hPTHrP peptide and/or analog in combination with calcium and/or vitamin D.

In some embodiments, the present invention provides a method for increasing bone mass in men suffering from primary or hypogonadal osteoporosis comprising administering to a subject in need thereof a hPTH or hPTHrP peptide and/or analog. In some embodiments, the present invention provides a method for increasing bone mass in men suffering from primary or hypogonadal osteoporosis comprising administering to a subject in need thereof a hPTH or hPTHrP peptide and/or analog in combination with calcium and/or vitamin D.

In some embodiments, the present invention provides a method for treating glucocorticoid-induced osteoporosis comprising administering to a subject in need thereof a hPTH or hPTHrP peptide and/or analog. In some embodiments, the present invention provides a method for treating glucocorticoid-induced osteoporosis comprising administering to a subject in need thereof a hPTH or hPTHrP peptide and/or analog in combination with calcium and/or vitamin D.

In certain embodiments, peptides and/or analogs of the present invention, or a pharmaceutically acceptable composition thereof, are administered in combination with one or more additional therapeutic agents.

In some embodiments, provided hPTH or hPTHrP peptides and/or analogs, or a pharmaceutical composition thereof, are administered in combination with one or more antiproliferative or chemotherapeutic agents. In some embodiments, provided hPTH or hPTHrP peptides and/or analogs, or a pharmaceutical composition thereof, are administered in combination with one or more antiproliferative or chemotherapeutic agents selected from any one or more of Abarelix, aldesleukin, Aldesleukin, Alemtuzumab, Alitretinoin, Allopurinol, Altretamine, Amifostine, Anastrozole, Arsenic trioxide, Asparaginase, Azacitidine, BCG Live, Bevacuzimab, Fluorouracil, Bexarotene, Bleomycin, Bortezomib, Busulfan, Calusterone, Capecitabine, Camptothecin, Carboplatin, Carmustine, Celecoxib, Cetuximab, Chlorambucil, Cladribine, Clofarabine, Cyclophosphamide, Cytarabine, Dactinomycin, Darbepoetin alfa, Daunorubicin, Denileukin, Dexrazoxane, Docetaxel, Doxorubicin (neutral), Doxorubicin hydrochloride, Dromostanolone Propionate, Epirubicin, Epoetin alfa, Erlotinib, Estramustine, Etoposide Phosphate, Etoposide, Exemestane, Filgrastim, floxuridine fludarabine, Fulvestrant, Gefitinib, Gemcitabine, Gemtuzumab, Goserelin Acetate, Histrelin Acetate, Hydroxyurea, Ibritumomab, Idarubicin, Ifosfamide, Imatinib Mesylate, Interferon Alfa-2a, Interferon Alfa-2b, Irinotecan, Lenalidomide, Letrozole, Leucovorin, Leuprolide Acetate, Levamisole, Lomustine, Megestrol Acetate, Melphalan, Mercaptopurine, 6-MP, Mesna, Methotrexate, Methoxsalen, Mitomycin C, Mitotane, Mitoxantrone, Nandrolone, Nelarabine, Nofetumomab, Oprelvekin, Oxaliplatin, Paclitaxel, Palifermin, Pamidronate, Pegademase, Pegaspargase, Pegfilgrastim, Pemetrexed Disodium, Pentostatin, Pipobroman, Plicamycin, Porfimer Sodium, Procarbazine, Quinacrine, Rasburicase, Rituximab, Sargramostim, Sorafenib, Streptozocin, Sunitinib Maleate, Talc, Tamoxifen, Temozolomide, Teniposide, VM-26, Testolactone, Thioguanine, 6-TG, Thiotepa, Topotecan, Toremifene, Tositumomab, Trastuzumab, Tretinoin, ATRA, Uracil Mustard, Valrubicin, Vinblastine, Vincristine, Vinorelbine, Zoledronate, or Zoledronic acid.

Other examples of agents the compounds of this invention may also be combined with include, without limitation: treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

In certain embodiments, hPTH or hPTHrP peptides and/or analogs of the present invention, or a pharmaceutically acceptable composition thereof, are administered in combination with a monoclonal antibody or an siRNA therapeutic.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another.

The amount of both, an inventive peptide and/or analog and an additional therapeutic agent (in those compositions which comprise an additional therapeutic agent as described above)) that may be combined with the carrier materials to produce a single dosage form will vary depending upon the host treated and the particular mode of administration. In some embodiments, compositions of this invention are be formulated so that a dosage of between 0.0001-100 mg/kg body weight/day of an analog can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.001-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a therapeutic agent. Implantable devices coated with a compound of this invention are another embodiment of the present invention.

### Exemplification

All commercial materials (Aldrich, Fluka, Nova) were used without further purification. All solvents were reagent grade or HPLC grade (Fisher). Anhydrous THF, diethyl ether, CH₂Cl₂, toluene, and benzene were obtained from a dry solvent system (passed through column of alumina) and used without further drying. All reactions were performed under an atmosphere of pre-purified dry Ar(g). NMR spectra (¹H and ¹³C) were recorded on a Bruker Advance II 600 MHz or Bruker Advance DRX-500 MHz, referenced to TMS or residual solvent. Low-resolution mass spectral analyses were performed with a JOEL JMS-DX-303-HF mass spectrometer or Waters Micromass ZQ mass spectrometer. Analytical TLC was performed on E. Merck silica gel 60 F254 plates and flash column chromatography was performed on E. Merck silica gel 60 (40-63 mm). Yields refer to chromatographically pure compounds.

HPLC: All separations involved a mobile phase of 0.05% TFA (v/v) in water (solvent A)/0.04% TFA in acetonitrile (solvent B). LCMS analyses were performed using a Waters 2695 Separations Module and a Waters 996 Photodiode Array Detector equipped with Varian Microsorb 100-5, C18 150×2.0 mm and Varian Microsorb 300-5, C4 250×2.0 mm columns at a flow rate of 0.2 mL/min. UPLC-MS analyses were performed using a Waters Acquity™ Ultra Preformance LC system equipped with Acquity UPLC® BEH C18, 1.7 μl, 2.1×100 mm, Acquity UPLC® BEH C8, 1.7 μl, 2.1×100 mm, Acquity UPLC® BEH 300 C4, 1.7 μl, 2.1×100 mm columns at a flow rate of 0.3 mL/min. Preparative separations were performed using a Ranin HPLC solvent delivery system equipped with a Rainin UV-1 detector and Varian Dynamax using Varian Microsorb 100-5, C18 250×21.4 mm and Varian Microsorb 300-5, C4 250×21.4 mm columns at a flow rate of 16.0 mL/min.

Solid Phase Peptide Synthesis (SPPS).

Automated peptide synthesis was performed on an Applied Biosystems Pioneer continuous flow peptide synthesizer. Peptides were synthesized under standard automated Fmoc protocols (HATU, DIEA, DMF). The deblocking solution was a mixture of 100/5/5 of DMF/piperidine/DBU (100/5/5). The following Fmoc amino acids from NovaBiochem were employed: Fmoc-Ala-OH, Fmoc-Arg(Pbf)-OH, Fmoc-Asn(Trt)-OH, Fmoc-Asp(OtBu)-OH, Boc-Thz-OH, Fmoc-Glu(OtBu)-OH, Fmoc-Gln(Trt)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Val-OH. Upon completion of automated synthesis on a 0.05 mmol scale, the peptide resin was washed with DCM. Cleavage was carried out using AcOH/TFE/DCM (1:1:8) or TFA/TIS/H₂O (95:2.5:2.5). The resin was removed by filtration, and the resulting solution was concentrated. The residue was precipitated with ether and centrifuged. The pellet was resuspended in acetonitrile/H₂O (1:1) and lyophilized.

CD spectra were obtained on an Aviv 410 circular dichroism spectropolarimeter. Protein concentrations were determined based on the extinction coefficient, calculated according to the number of Trp residue. The solvent for all experiments were 1:1 CH₃CN:H₂O. Spectra were collected with a 1 mm path length cuvette at protein concentration of 14 μM and 7 μM.

### Example 1

### Synthesis of hPTH (1-84)

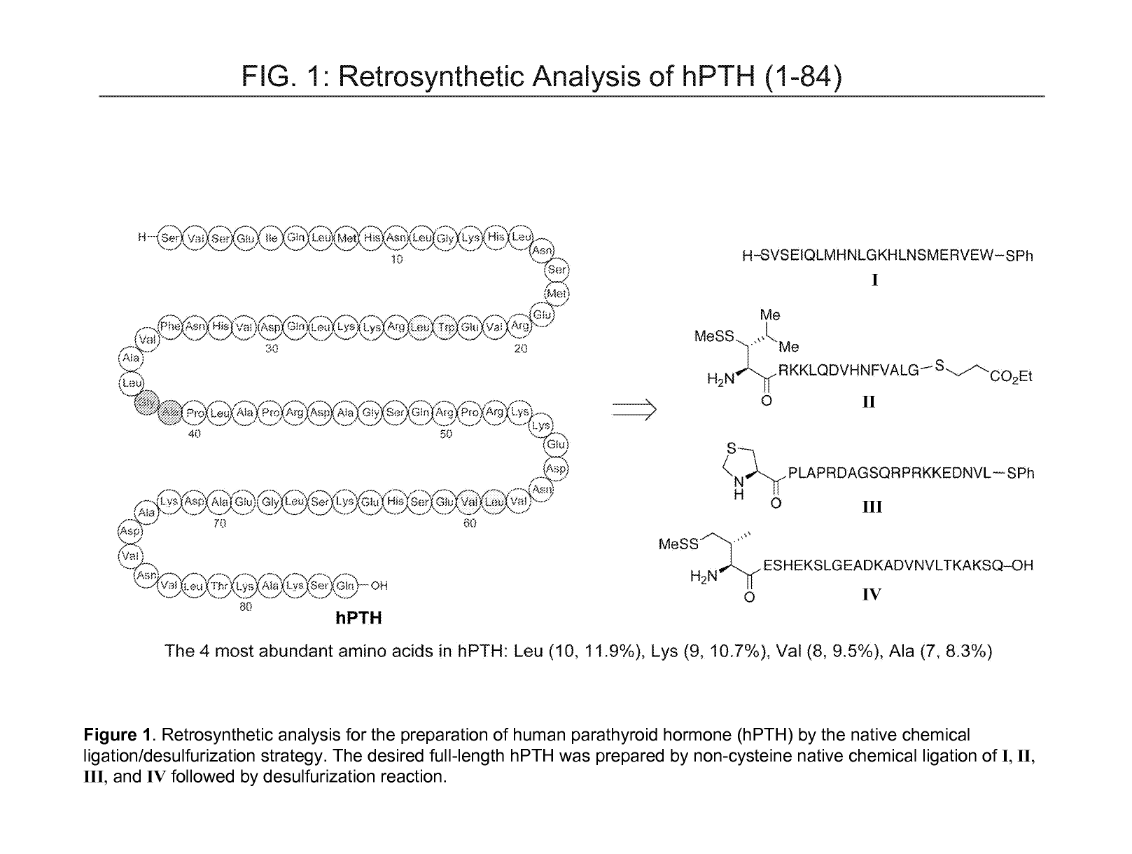

The primary structure of hPTH is shown in FIG. 1. On the basis of its amino acid sequence, the hPTH polypeptide chain can be assembled by a convergent strategy from four fragments, hPTH (1-23) I, hPTH (24-38) II, hPTH (39-59) III, and hPTH (60-84) IV. Each peptide fragment contains 23 amino acid residues, 15 residues, 21 residues, and 25 residues, respectively, and is thus readily made by solid phase peptide synthesis. The fragments are joined together through the use of three of the most abundant amino acids in hPTH, Leu24, Ala39, and Va160 (FIG. 1).

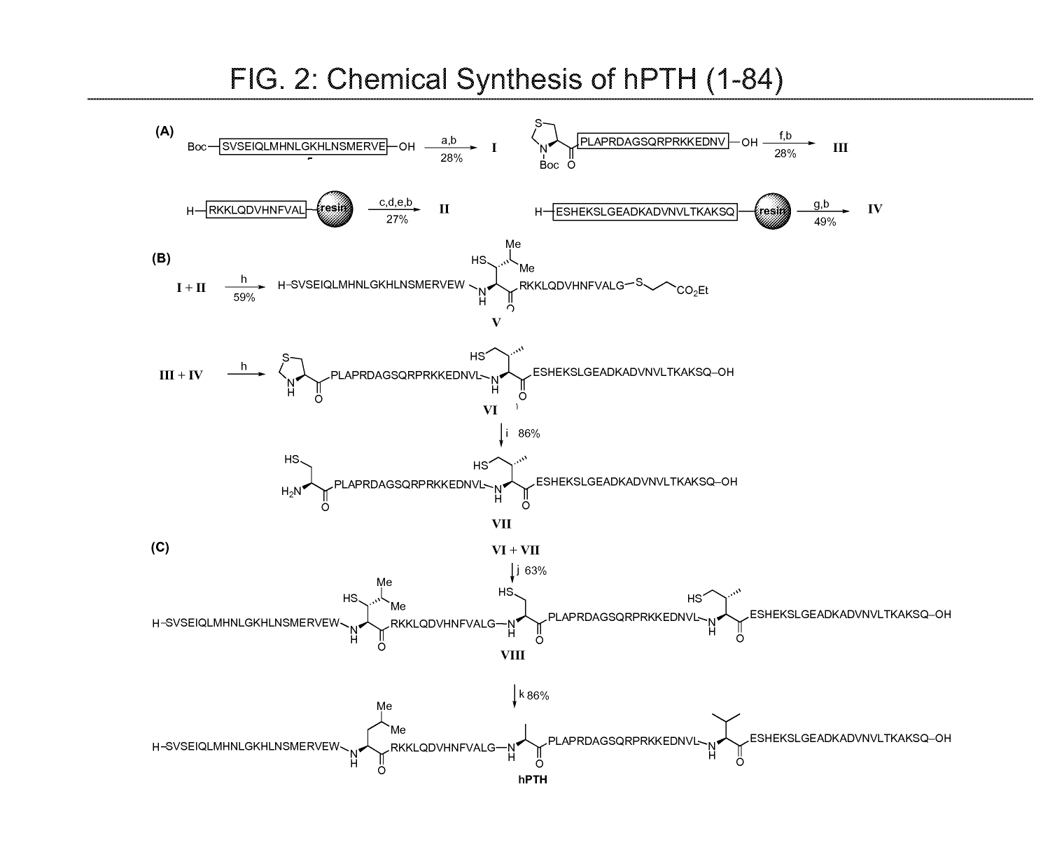

The synthesis of hPTH is shown in FIG. 2. Fully protected peptides were manually synthesized by Fmoc chemistry on a 0.05 mmol scale. The leucine and valine surrogates were attached to the N-termini of the fully protected peptides by HATU. The peptide fragments bearing C-terminal thioesters were prepared from the fully protected peptides using the EDCI-mediated amide formation reaction under the non-epimerizing conditions developed by Sakakibara and co-workers. Selective leucine ligation of fragment I thioester and fragment II was completed in 9 h to afford peptide V in 59% yield. The reaction of fragment III and fragments IV was carried out in pH 7.5 guanidine buffer for 5 h to give peptide VI. After ligation was completed, the thiazolidine in peptide VI was converted into N-terminal cysteine in one-pot by treatment with methoxylamine.HCl at pH 4.0, giving an 86% yield over two steps (FIG. 2B). After these syntheses, ligation of peptide V thioester and VII in the presence of 200 mM (4-methoxyphenyl acetic acid (MPAA) catalyst generates VIII in 63% yield. The desulfurization of VIII was completed in 2 h and yielded the final full-length product. Purification by HPLC provided pure hPTH in 86% yield.

Synthesis of Peptide Thiophenyl Ester I:

H-SVSEIQLMHNLGKHLNSMERVEW-SPh I

The fully protected peptidyl acid was prepared by SPPS using the general procedure described above. After cleavage, 156.4 mg crude peptide was obtained (68% yield).

The fully protected peptidyl acid (71.7 mg, 15.8 μM, 1.1 equiv) and HCl.H-Trp-SPh (4.8 mg, 14.4 μM, 1.0 equiv) in CHCl₃/TFE (v/v=3/1, 620 μL) was cooled to −10° C. HOOBt (2.6 mg, 15.8 μM, 1.1 equiv) and EDCI (2.8 μL, 15.8 μM, 1.1 equiv) were added. The reaction mixture was stirred at room temperature for 3 h. The solvent was then blown off under a gentle N₂ stream and TFA/H₂O/TIS (95:2.5:2.5) was added. After deprotection for 45 min, TFA was blown off and the oily residue was triturated with diethyl ether. The precipitate was pelleted and the ether was subsequently decanted. The resulting solid was purified by HPLC to give 11.5 mg fragment I, 28% yield. Chemical Formula: C₁₂₄H₁₉₃N₃₅O₃₅S₃, Expected Mass: 2828.36, [M+2H]²⁺ m/z=1415.18, [M+3H]³⁺ m/z=943.79.

Synthesis of Thioleucine-Containing Peptide Alkyl Thioester II.

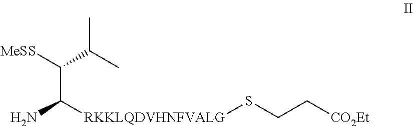

The peptide resin from the Fmoc SPPS (6.49 μmol, 1.0 equiv) was mixed with Boc-Leu(SSMe)-OH (2.0 mg, 6.49 μmol, 1.0 equiv), HATU (7.6 mg, 19.5 μmol, 3.0 equiv) and DIEA (6.8 μL, 39.0 μmol, 6.0 equiv) in DMF (200 μL) and stirred at room temperature for 10 min. The resin was washed with DMF, DCM and MeOH several times and dried under vacuum. The dried resin was cleaved by treatment with AcOH/TFE/DCM (1:1:8) for 2×1 hour to yield the fully protected peptidyl acid.

The above crude peptidyl acid (6.49 μM, 1.0 equiv) and HCl.H-Gly-3-thiopropionic acid ethyl ester (7.79 μM, 1.2 equiv) in CHCl₃/TFE (v/v=3/1, 435 μL) was cooled to −10° C. HOOBt (6.49 μM, 1.0 equiv) and EDCI (6.49 μM, 1.0 equiv) were added. The reaction mixture was stirred at room temperature for 3.5 h. The solvent was then blown off under a gentle N₂ stream and TFA/H₂O/TIS (95:2.5:2.5) was added. After deprotection for 20 min, TFA was blown off and the oily residue was triturated with diethyl ether. The precipitate was pelleted and the ether was subsequently decanted. The resulting solid was purified by HPLC to give 3.7 mg thioester II, 30% yield (calculated based on the resin weight). Chemical Formula: C₈₅H₁₄₂N₂₄O₂₁S₃, Expected Mass: 1930.99, [M+2H]²⁺ m/z=966.50.

Synthesis of Peptide Thiophenyl Ester III.

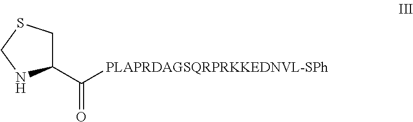

The fully protected peptidyl acid was prepared by SPPS using the general procedure described above. After cleavage, 45.5 mg crude peptide was obtained (23% yield).

The fully protected peptidyl acid (45.5 mg, 11.3 μM, 1.1 equiv) and HCl.H-Leu-SPh (2.7 mg, 10.3 μM, 1.0 equiv) in CHCl₃/TFE (v/v=3/1, 440 μL) was cooled to −10° C. HOOBt (1.8 mg, 11.3 μM, 1.1 equiv) and EDCI (2.0 μL, 11.3 μM, 1.1 equiv) were added. The reaction mixture was stirred at room temperature for 3 h. The solvent was then blown off under a gentle N₂ stream and TFA/H₂O/TIS (95:2.5:2.5) was added. After deprotection for 45 min, TFA was blown off and the oily residue was triturated with diethyl ether. The precipitate was pelleted and the ether was subsequently decanted. The resulting solid was purified by HPLC to give 7.2 mg thiophenyl ester III, 28% yield. Chemical Formula: C₁₀₅H₁₇₂N₃₄O₃₀S₂, Expected Mass: 2453.24, [M+2H]²⁺ m/z=1227.62, [M+3H]³⁺ m/z=818.75.

Synthesis of Thiovaline-containing Peptide IV.

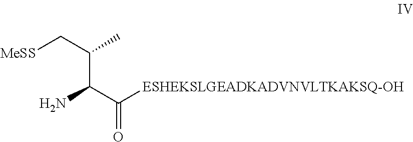

The peptide resin from the Fmoc SPPS (6.64 μmol, 1.0 equiv) was mixed with Boc-Val(SSMe)-OH (2.0 mg, 6.64 μmol, 1.0 equiv), HATU (7.6 mg, 19.9 μmol, 3.0 equiv) and DIEA (6.9 μL, 39.8 μmol, 6.0 equiv) in DMF (200 μL) and stirred at room temperature for 10 min. The resin was washed with DMF, DCM and MeOH several times and dried under vacuum. The peptide was cleaved and deprotected by treatment with TFA/H₂O/TIS (95:2.5:2.5) for 1 h 10 min. TFA was then blown off and the oily residue was triturated with diethyl ether. The precipitate was pelleted and the ether was subsequently decanted. The resulting solid was purified by HPLC to give 8.9 mg thioester IV, 49% yield (calculated based on the resin weight). Chemical Formula: C₁₁₄H₁₉₃N₃₃O₄₂S₂, Expected Mass: 2760.34, [M+2H]²⁺ m/z=1381.17, [M+3H]³⁺ m/z=921.11.

Synthesis of Peptide V.

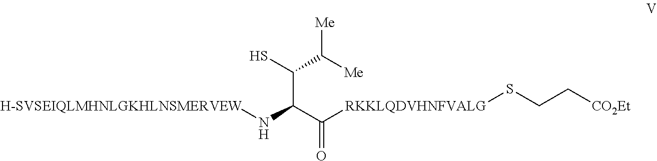

The synthesis of V was carried out under kinetically controlled ligation conditions. Peptide I (6.1 mg, 2.2 μmol, 1.1 equiv) and peptide II (3.7 mg, 1.9 μmol, 1.0 equiv) were dissolved in ligation buffer (600 μL, 6 M Gdn.HCl, 100 mM Na₂HPO₄, 50 mM TCEP, pH 7.5). The reaction mixture was stirred at room temperature for 9 h. The reactions were monitored by LC-MS and purified directly by HPLC to give 5.2 mg ligated peptide V, 59% yield. As estimated by LC-MS analysis, the ratio between the cyclization product of II and the ligation product V is 1:10. Chemical Formula: C₂₀₂H₃₂₇N₅₉O₅₆S₄, Expected Mass: 4603.34, [M+2H]²⁺ m/z=2302.67, [M+3H]³⁺ m/z=1535.45, [M+4H]⁴⁺ m/z=1151.84, [M+5H]⁵⁺ m/z=921.67.

Synthesis of Ligated Peptide VI.

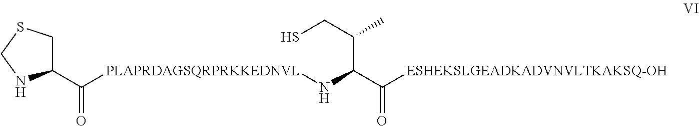

Peptide III (2.7 mg, 1.1 μmol, 1.6 equiv) and peptide IV (1.8 mg, 0.67 μmol, 1.0 equiv) were dissolved in ligation buffer (300 μL, 6 M Gdn.HCl, 100 mM Na₂HPO₄, 50 mM TCEP, pH 7.5). The reaction mixture was stirred at room temperature for 9 h. The reactions were monitored by LC-MS and the crude peptide VI was deprotected directly without further purification.

Synthesis of Peptide VII.

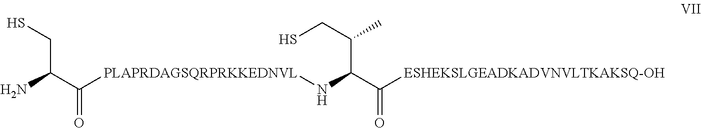

The Thz group was converted to cysteine by addition of 0.2 M methoxylamine HCl at pH 4.0. The reaction mixture was stirred at room temperature for 5 h. The reactions were monitored by LC-MS and purified directly by HPLC to give 2.9 mg deprotected peptide VII, 86% yield. Chemical Formula: C₂₁₁H₃₅₇N₆₇O₇₂S₂, Expected Mass: 5045.58, [M+3H]³⁺ m/z=1682.86, [M+4H]⁴⁺ m/z=1262.40, [M+5H]⁵⁺ m/z=1010.12, [M+6H]⁷⁺ m/z=841.93, [M+7H]⁵⁺ m/z=721.81.

Synthesis of Ligated Peptide VIII.

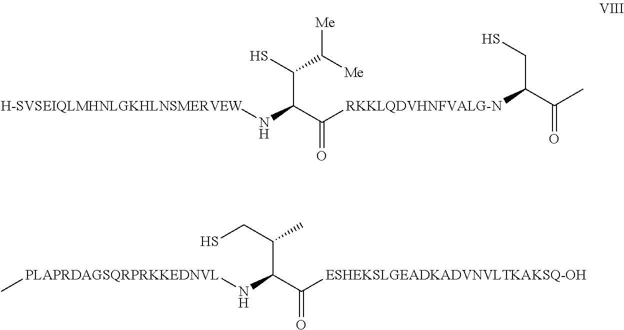

Peptide V (1.1 mg, 0.24 μmol, 1.1 equiv) and peptide VII (1.1 mg, 0.22 μmol, 1.0 equiv) were dissolved in ligation buffer (100 μL, 6 M Gdn.HCl, 300 mM Na₂HPO₄, 200 mM MPAA, 20 mM TCEP, pH 7.9). The reaction mixture was stirred at room temperature for 4 h. The reactions were monitored by LC-MS and purified directly by HPLC to give 1.3 mg ligated peptide VIII, 59% yield. Chemical Formula: C₄₀₈H₆₇₄N₁₂₆O₁₂₆S₅, Expected Mass: 9514.88, [M+5H]⁵⁺ m/z=1903.98, [M+6H]⁶⁺ m/z=1586.81, [M+7H]⁷⁺ m/z=1360.27, [M+8H]⁸⁺ m/z=1190.36, [M+9H]⁹⁺ m/z=1058.21, [M+10H]¹⁰⁺ m/z=952.49, [M+11H]¹¹⁺ m/z=865.99, [M+12H]¹²⁺ m/z=793.91, [M+13H]¹³⁺ m/z=732.91.

Synthesis of Desulfurized Peptide hPTH.

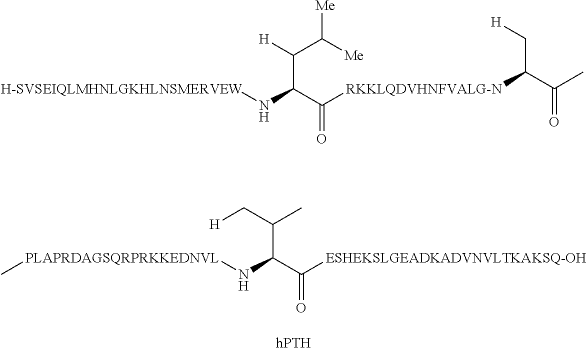

To a solution of the purified ligated peptide VIII (0.7 mg) in degassed CH₃CN/H₂O (v/v=1:1, 0.2 ml) were added 0.2 ml of 0.5 M bond-Breaker® TCEP solution (Pierce), 0.02 ml of 2-methyl-2-propanethiol and 0.2 ml of radical initiator (0.1 M in H₂O). The reaction mixture was stirred at 37° C. for 2 h. The reactions were monitored by LC-MS and purified directly by HPLC to give 0.6 mg hPTH, 86%. Chemical Formula: C₄₀₈H₆₇₄N₁₂₆O₁₂₆S₂, Expected Mass: 9418.96, [M+5H]⁵⁺ m/z=1884.79, [M+6H]⁶⁺ m/z=1570.83, [M+7H]⁷⁺ m/z=1346.57, [M+8H]⁸⁺ m/z=1178.37, [M+9H]⁹⁺ m/z=1047.55, [M+10H]¹⁰⁺ m/z=942.90, [M+11H]¹¹⁺ m/z=857.27, [M+12H]¹²⁺ m/z=785.91, [M+13H]¹³⁺ m/z=725.54.

### Example 2

### Synthesis of [Nle^{8,18}]hPTH (1-84)

Synthesis of Peptide Phenol Ester IX:

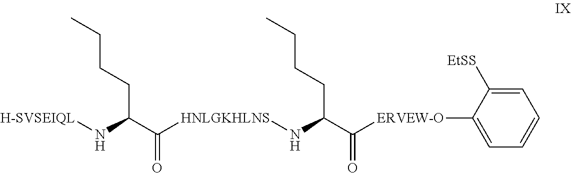

The fully protected peptidyl acid was prepared by solid-phase peptide synthesis (SPPS) using the general procedure described above. After cleavage, 151.0 mg crude peptide was obtained (66% yield).

The fully protected peptidyl acid (87.8 mg, 19.3 μM, 1.1 equiv) and HCl.H-Trp-Ar (7.2 mg, 17.5 μM, 1.0 equiv) in CHCl₃/TFE (v/v=3/1, 1 mL) was cooled to −10° C. HOOBt (3.1 mg, 19.3 μM, 1.1 equiv) and EDCI (3.4 μL, 19.3 μM, 1.1 equiv) were added. The reaction mixture was stirred at room temperature for 3 h. The solvent was then blown off under a gentle N₂ stream and 7 mL of TFA/H₂O/TIS (95:2.5:2.5) was added. After deprotection for 45 min, TFA was blown off and the oily residue was triturated with 5 mL of diethyl ether. The precipitate was pelleted and the ether was subsequently decanted. The resulting solid was purified by HPLC to give 11.0 mg phenol ester IX, 22% yield. Chemical Formula: C₁₂₈H₂₀₁N₃₅O₃₆S₂; Expected Mass: 2868.44, [M+2H]²⁺ m/z=1435.22, [M+3H]³⁺ m/z=957.15, [M+4H]⁴⁺ m/z=718.11.

Synthesis of Peptide X:

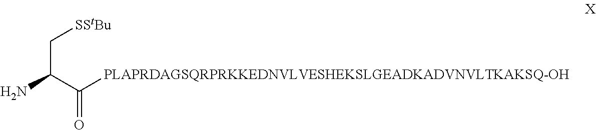

The fully deprotected peptidyl acid X was prepared by SPPS using the general procedure described above. After HPLC purification, 28.1 mg peptide was obtained (11% yield). Chemical Formula: C₂₁₅H₃₆₅N₆₇O₇₂S₂, Expected Mass: 5101.64, [M+3H]³⁺ m/z=1701.55, [M+4H]⁴⁺ m/z=1276.41, [M+5H]⁵⁺ m/z=1021.33, [M+6H]⁶⁺ m/z=851.27, [M+7H]⁷⁺ m/z=729.81, [M+8H]⁸⁺ m/z=638.70.

Ligated Peptide XI:

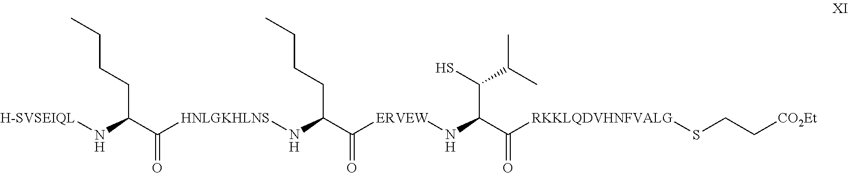

The synthesis of XI was carried out under kinetically controlled ligation conditions. Peptide IX (5.3 mg, 1.85 μmol, 1.27 equiv) and peptide II (2.8 mg, 1.45 μmol, 1.0 equiv) were dissolved in ligation buffer (600 μL, 6 M Gdn.HCl, 100 mM Na₂HPO₄, 50 mM TCEP, pH 7.5). The reaction mixture was stirred at room temperature for 2 h. The reactions were monitored by LC-MS and purified directly by HPLC to afford 1.3 mg ligated peptide XI, 20% yield. Chemical Formula: C₂₀₄H₃₃₁N₅₉O₅₆S₂, Expected Mass: 4567.43, [M+3H]³⁺ m/z=1523.48, [M+4H]⁴⁺ m/z=1142.86, [M+5H]⁵⁺ m/z=914.49, [M+6H]⁶⁺ m/z=762.24.

Ligated Peptide XII:

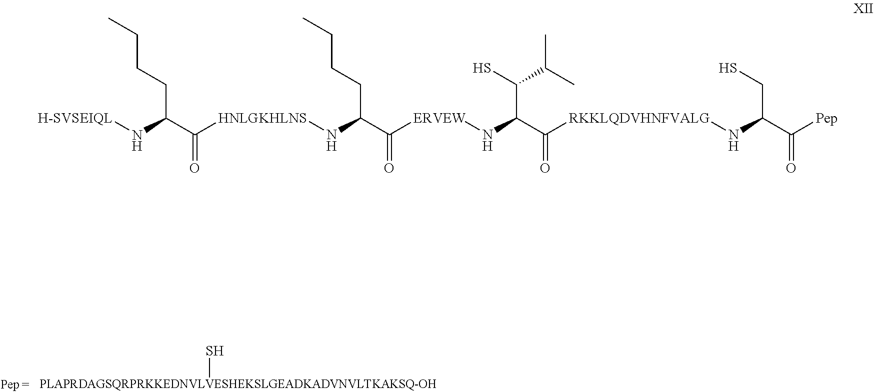

Peptide XI (2.0 mg, 0.438 μmol, 1.1 equiv) and peptide X (2.0 mg, 0.398 μmol, 1.0 equiv) were dissolved in ligation buffer (200 μL, 6 M Gdn.HCl, 300 mM Na₂HPO₄, 200 mM MPAA, 20 mM TCEP, pH 7.9). The reaction mixture was stirred at room temperature for 1 h. The reactions were monitored by LC-MS and purified directly by HPLC to give 1.6 mg ligated peptide XII, 43% yield.

Desulfurized Peptide [Nle^{8,18}]hPTH(1-84) (XIII):

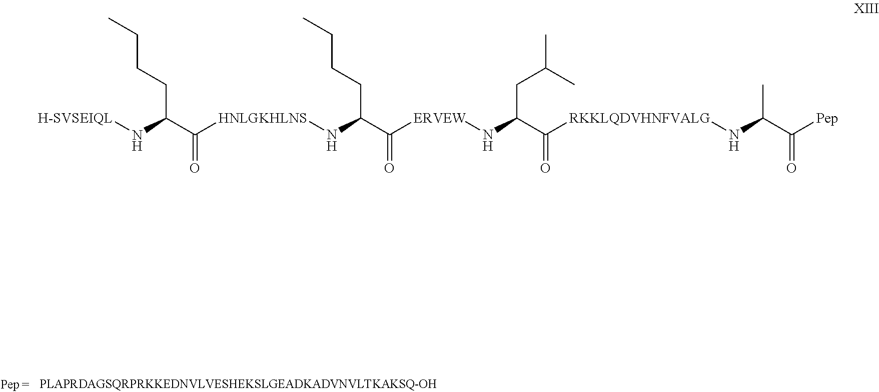

To a solution of the purified ligated peptide XII (1.6 mg) in degassed CH₃CN/H₂O (v/v=1:1, 0.2 ml) were added 0.2 ml of 0.5 M bond-Breaker® TCEP solution (Pierce), 0.02 ml of 2-methyl-2-propanethiol and 0.2 ml of radical initiator (0.1 M in H₂O). The reaction mixture was stirred at 37° C. for 2 h. The reactions were monitored by LC-MS and purified directly by HPLC to give 0.9 mg [Nle^{8,18}]hPTH(1-84) (XIII), 57% yield. Chemical Formula: C₄₁₀H₆₇₈N₁₂₆O₁₂₆, Expected Mass: 9383.05, [M+5H]⁵⁺ m/z=1877.61, [M+6H]⁶⁺ m/z=1564.84, [M+7H]⁷⁺ m/z=1341.44, [M+8H]⁸⁺ m/z=1173.88, [M+9H]⁹⁺ m/z=1043.56, [M+10H]¹⁰⁺ m/z=939.30, [M+11H]¹¹⁺ m/z=854.00, [M+12H]¹²⁺ m/z=782.92, [M+13H]ⁿ⁺ m/z=722.77, [M+14H]¹⁴⁺ m/z=626.54.

### Example 3

### Synthesis of [Nle^{8,18}]hPTH (1-37)

Synthesis of Peptide XIV:

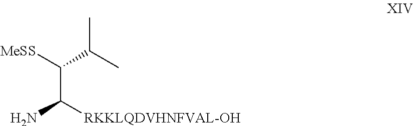

The peptide resin from the Fmoc SPPS (9.12 μmol, 1.0 equiv) was mixed with Boc-Leu(SSMe)-OH (4.8 mg, 15.50 μmol, 1.7 equiv), HATU (17.3 mg, 45.6 μmol, 5.0 equiv) and DIEA (15.9 μL, 91.2 μmol, 10.0 equiv) in DMF (500 μL) and stirred at room temperature for 10 min. The resin was washed with DMF, DCM and MeOH several times and dried under vacuum. The dried resin was treated with TFA/TIS/H₂O (95:2.5:2.5) for 40 min, TFA was blown off by N₂ and the oily residue was triturated with diethyl ether. The precipitate was pelleted and the ether was subsequently decanted. The resulting solid was purified by HPLC to give 8.2 mg peptide XIV, 51% yield (calculated based on the resin).

Synthesis of Peptide XV:

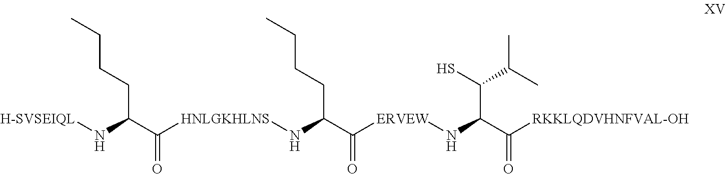

Peptide IX (1.8 mg, 0.628 μmol, 1.5 equiv) and peptide XIV (0.74 mg, 0.418 μmol, 1.0 equiv) were dissolved in ligation buffer (167 μL, 6 M Gdn.HCl, 100 mM Na₂HPO₄, 50 mM TCEP, pH 7.5). The reaction mixture was stirred at room temperature for 2.5 h. The reaction was monitored by LC-MS and quenched with 1 mL of CH₃CN/H₂O/AcOH (1:1:5%) solution. Purification using HPLC afforded 0.8 mg of peptide XV (44%). Chemical Formula: C₁₉₇H₃₂₀N₅₈O₅₄S, Expected Mass: 4394.38, [M+3H]³⁺ m/z=1465.79, [M+4H]⁴⁺ m/z=1099.59, [M+5H]⁵⁺ m/z=879.88, [M+6H]⁶⁺ m/z=733.40.

Desulfurized Peptide [Nle^{8,18}]hPTH(1-37) (XVI):

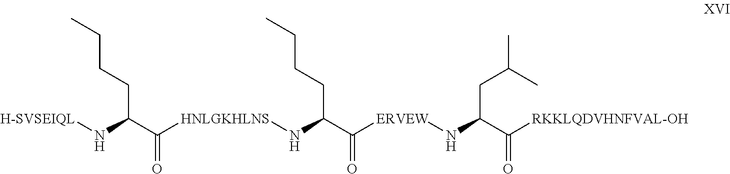

To a solution of the purified ligated peptide XV (0.8 mg) in degassed CH₃CN/H₂O (v/v=1:1, 0.2 ml) were added 0.2 ml of 0.5 M bond-Breaker® TCEP solution (Pierce), 0.02 ml of 2-methyl-2-propanethiol and 0.2 ml of radical initiator (0.1 M in H₂O). The reaction mixture was stirred at 37° C. for 4 h. The reactions were monitored by LC-MS and purified directly by HPLC to afford 0.3 mg [Nle^{8,18}]hPTH(1-37) (XVI), 38%. Chemical Formula: C₁₉₇H₃₂₀H₅₈O₅₄, Expected Mass: 4362.41, [2M+5H]⁵⁺ m/z=1745.96, [M+3H]³⁺ m/z=1455.14, [M+4H]⁴⁺ m/z=1091.60, [M+5H]⁵⁺ m/z=873.48, [M+6H]⁶⁺ m/z=728.07.

### Example 4

### Synthesis of hPTHrP (XXXVII)

Synthesis of Peptide XXX:

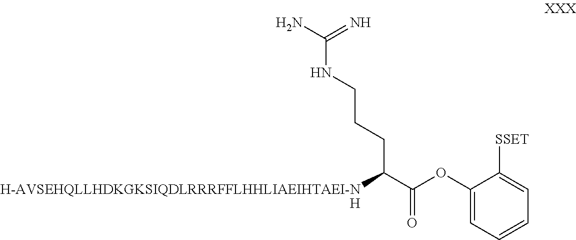

The fully protected peptide H-AVSEHQLLHDKGKSIQDLRRRFFLHHLIAEIHTAEIR-OH (510.00 mg, 67.6 μmol, 1.0 eq) was mixed with (2 S)-1-(2-(ethylsulfinothioyl)phenoxy)-1-oxo-5-(3-((2,2,4,6,7-pentamethyl-2,3-dihydrabenzofuran-5-yl)sulfonyl)guanidino)pentan-2-aminium chloride (85.37 mg, 2.0 eq) and HOOBt (22.06 mg, 2.0 eq) in the solvent (1.5 ml, CHCl₃/TFE=3:1 v/v) and then cooled down to −10° C. To the mixture was added slowly EDC (23.9 μl, 2.0 eq). The mixture was subsequently allowed to warm to 23° C. and stirred for 3 h, monitored with UPLC. The resulting mixture was treated with 5% HOAc (2.0 ml) in water and the organic layer was separated. The organic layer then was injected in a cocktail B solution (30.0 ml) and stirred for 1.5 h. After that, the solution was then concentrated under N₂ stream and the crude product was precipitated by pouring in cold diethyl ether (30.0 ml). The suspension was centrifuged and the upper ether layer was decanted. The precipitated was purged with diethyl ether (2×30.0 ml) and the precipitated was dissolved in aq. MeCN (20.0 ml) and lypholized. The resulting crude product was further purified with preparative HPLC to afford 33.12 mg of peptide XXX (11% yield). Chemical Formula: C₂₀₅H₃₂₅N₆₃O₅₃S₂, Expected Mass 4581.41, [M+4H]⁴⁺ m/z=1146.9, [M+5H]⁵⁺ m/z=917.8.

Synthesis of Peptide XXXI:

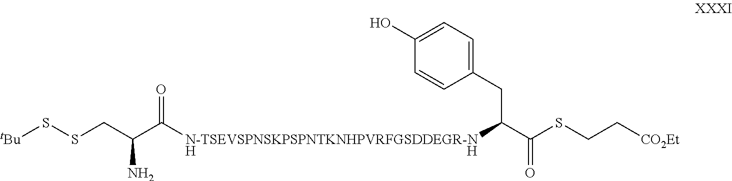

The fully protected peptide H-TSEVSPNSKPSPNTKNHPVRFGSDDEGRY-OH (147.0 mg, 25.6 μmol, 1.0 eq) was mixed with (S)-ethyl 3-((2-amino-3-(4-(tert-butoxy)phenyl)propanoyl)thio)propanoate (18.12 mg, 2.0 eq) and HOOBt (7.96 mg, 2.0 eq) in the solvent (0.25 ml, CHCl₃/TFE=3:1 v/v) and then cooled down to −10° C. To the mixture was added slowly EDC (9.1 μl, 2.0 eq). The mixture was subsequently allowed to warm to 23° C. and stirred for 3 h, monitored with UPLC. The resulting mixture was treated with 5% HOAc (0.5 ml) in water and the organic layer was separated. The organic layer then was injected in a cocktail B solution (20.0 ml) and stirred for 1.5 h. After that, the solution was then concentrated under N₂ stream and the crude product was precipitated by pouring in cold diethyl ether (20.0 ml). The suspension was centrifuged and the upper ether layer was decanted. The precipitated was purged with diethyl ether (2×20.0 ml) and the precipitated was dissolved in aq. MeCN (15.0 ml) and lypholized. The resulting crude product was further purified with preparative to afford 25.34 mg of peptide XXXI (29% yield). Chemical Formula: C₁₄₇H₂₂₉N₄₃O₅₁S₃, Expected Mass 3508.58, [M+3H]³⁺ m/z=1170.9, [M+4H]⁴⁺ m/z=878.9.

Synthesis of Peptide XXXII:

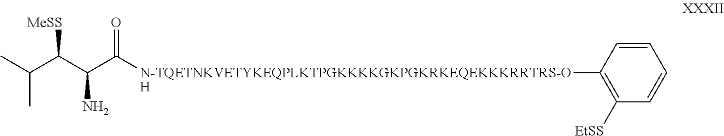

The peptide resin from the Fmoc SPPS (0.10 mmol, 1.0 eq) was mixed with Boc-Leu(SSMe)-OH (31.91 mg, 1.0 eq), HATU (114.02 mg, 3.0 eq), and DIEA (104 μl, 6.0 eq) in DMF (1.0 ml) and stirred at 23° C. for 10 min. The reasin was washed with DMF, DCM, and MeOH several times and dried under vacuum. The resin was cleaved by treatment with AcOH/TFE/DCM (1:1:8) for 2×1 hour to yield the fully protected peptidyl acid.

The fully protected peptidyl acid (266.80 mg, 29.7 μmol, 1.0 eq) and (2S)-2-(ethylsulfinothioyl)phenyl 2-amino-3-(tert-butoxy)propanoate (19.58 mg, 2.0 eq) was dissolved in solvents (594 μl, CHCl₃/TFE=3:1 v/v). To this mixture was added HOOBt (9.69 mg, 2.0 eq). The mixture was then sonicated and cooled to −10° C. To the mixture was added slowly EDC (11.0 μl, 2.0 eq) with stirring. The mixture was subsequently allowed to warm to 23° C. and stirred for 3 h, monitored with UPLC. The resulting mixture was treated with 5% HOAc in water (1.0 ml) and the organic layer was separated. The organic layer then was injected in a cocktail B solution (30.0 ml) and stirred for 1.5 h. After that, the solution was then concentrated under N₂ stream and the crude product was precipitated by pouring in cold diethyl ether (30.0 ml). The suspension was centrifuged and the upper ether layer was decanted. The precipitated was purged with diethyl ether twice (30.0 ml each) and the precipitated was dissolved in aq. MeCN (1:1 v/v, 20 ml) and lypholized. The resulting crude product was further purified with HPLC to afford 46.46 mg of peptide XXXII (9% overall yield). Chemical Formula: C₂₂₅H₃₉₁N₇₁O₆₄S₄, Expected Mass 5239.84, [M+4H]⁴⁺ m/z=1311.9, [M+5H]⁵⁺ m/z=1049.5.

Synthesis of Peptide XXXIII:

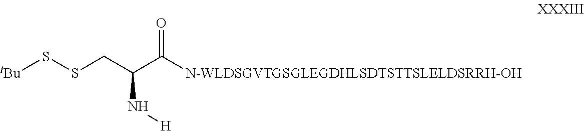

The synthesis of XXXIII was directly accomplished via Fmoc-SPPS (0.05 mmol scale). Chemical Formula: C₁₄₅H₂₃₁N₄₃O₅₅S₂, Expected Mass 3518.60, [M+3H]³⁺ m/z=1174.3, [M+4H]⁴⁺ m/z=881.2.

Synthesis of Peptide XXXIV:

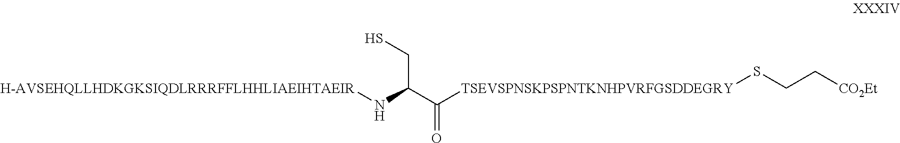

Peptide XXX (2.5 mg, 0.39 μmol, 1.00 eq) and peptide XXXI (1.54 mg, 0.44 μmol, 1.12 eq) were dissolved in aq MeCN and lyophilized. To the resulting starting materials was added ligation buffer (300 μl, 6 M Gdn.HCl, 100 mM Na₂HPO₄, 50 mM TCEP, pH 7.2). The mixture was stirred under argon at 23° C. for 3 h, monitored with UPLC and then purified with preparative HPLC to afford 1.63 mg peptide XXXIV (49% yield). Chemical Formula: C₃₅₇H₆₀₂N₁₁₄O₁₁₈S₂, Expected Mass 8438.41, [M+11H]¹¹⁺ m/z=768.32, [M+12H]¹²⁺ m/z=845.39.

Synthesis of Peptide XXXV:

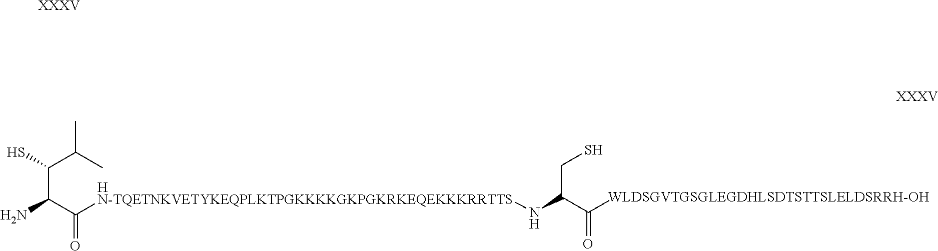

Peptide XXXII (3.53 mg, 0.77 μmol, 1.0 eq) and peptide XXXIII (2.70 mg, 0.77 μmol, 1.0 eq) were dissolved in ligation buffer (350 μl, 6 M Gdn.HCl, 100 mM Na₂HPO₄, 50 mM TCEP, pH 7.2). The mixture was stirred under argon at 23° C. for 3 h, monitored with UPLC and then purified with preparative HPLC to afford 3.35 mg peptide XXXV (56% yield). Chemical Formula: C₃₄₀H₅₃₆N₁₀₆O₁₀₃S₂, Expected Mass 7815.94, [M+5H]⁵⁺ m/z=1564.8, [M+6H]⁶⁺ m/z=1304.1.

Synthesis of Peptide XXXVI:

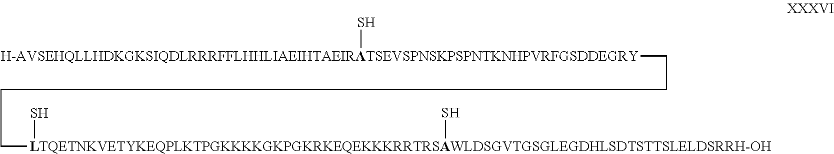

Ligation of peptide XXXIV and peptide XXXV was conducted under the kinetically controlled conditions. Peptide XXXIV (2.28 mg, 0.29 μmol, 1.0 eq) and peptide XXXV (2.95 mg, 0.35 μmol, 1.2 eq) were dissolved in ligation buffer (292 μl, 6 M Gdn.HCl, 300 mM Na₂HPO₄, 20 mM TCEP, 200 mM MPAA, pH 7.2). The mixture was stirred under argon at 23° C. for 16 h. The reaction was monitored with UPLC and then purified with preparative HPLC to afford 6.91 mg peptide XXXVI (containing TCEP for protection against oxidation). Chemical Formula: C₆₉₂H₁₁₂₈N₂₂₀O₂₁₉S₃, Expected Mass 16120.31, [M+14H]¹⁴⁺ m/z=1153.03, [M+15H]¹⁵⁺ m/z=1076.29, [M+16H]¹⁶⁺ m/z=1009.17, [M+17H]¹⁷⁺ m/z=949.88, [M+18H]¹⁸⁺ m/z=897.13.

Synthesis of Peptide XXXVII:

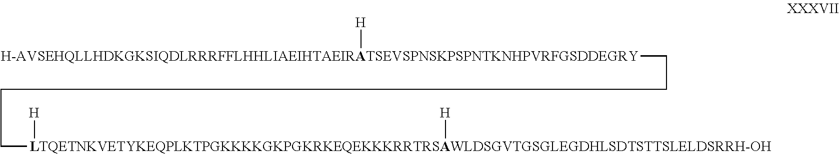

Peptide XXXVI was dissolved in buffer (1.4 ml, 6 M Gdn.HCl, 100 mM Na₂HPO₄, pH 7.2). To this buffer was added VA-044 (32.0 mg) and Bond Breaker (600 μl, 0.5 M solution of TCEP) and tBuSH (100 μl). The system was stirred under argon atmosphere at 37° C. for 2 h. Additional VA-044 (32.0 mg in 1.0 ml water) and tBuSH (100 μl) were added to the mixture and the mixture was stirred for additional 1 h. The reaction was monitored with LC-MS. The product was directly purified with preparative HPLC to afford 0.92 mg XXXVII (20% yield, over two steps). Chemical Formula: C₆₉₂H₁₁₂₈N₂₂₀O₂₁₉, Expected Mass 16024.39, [M+14H]¹⁴⁺ m/z=1147.26, [M+15H]¹⁵⁺ m/z=1071.15, [M+16H]¹⁶⁺ m/z=1003.39, [M+17H]¹⁷⁺ m/z=944.74, [M+18H]¹⁸⁺ m/z=892.76.

### Example 5

### In Vitro Assay of Parathyroid Hormone Analogs

Parathyroid hormone (PTH), via its receptor, the PTHR1 or PTHR, plays a critical role in maintaining normal blood concentrations of ionized calcium (Ca⁺⁺) and inorganic phosphate (Pi). Thus, in rapid response to a decrease in the blood Ca⁺⁺ concentration, PTH is secreted from the parathyroid glands and acts on bone to promote resorption of the mineralized matrix, and on kidney to promote reabsorption of Ca⁺⁺ from the glomerular filtrate. These coordinated actions in bone and kidney serve to maintain blood and fluid Ca⁺⁺ levels within a narrow range (˜1.2 mM±10%). The PTHR1 is a class B G protein-coupled receptor that signals mainly via the Gas/cAMP/PKA second messenger pathways.

### Analysis of PTH Receptor Binding Affinity of PTH Analogs

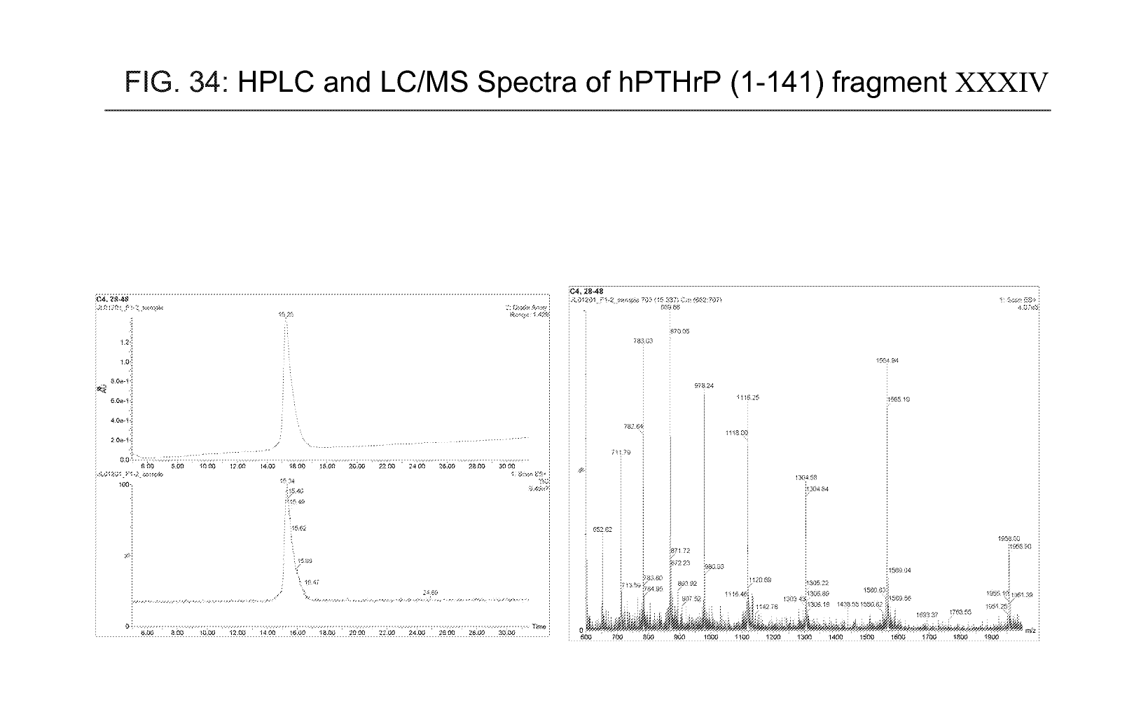

The capacities of the analogs to bind to the PTHR in a G protein-independent, conformation, R⁰, and a G protein-dependent conformation, RG, were assessed in membrane-based competition assays. Assays for R⁰ were performed using ¹²⁵I-PTH(1-34) tracer radioligand and in the presence of excess GTPγS. Under these R⁰ conditions, each analog bound with an affinity in the low- to mid-nanomolar range (IC₅₀s=4 nM to 40 nM; Log M=−8.4 to −7.4; FIG. 34A, Table 1). Assays for RG binding were performed using ¹²⁵I-M-PTH(1-15) tracer radioligand and membranes from cells expressing a high affinity, Gas mutant. Under these RG conditions, each analog bound with an affinity in the sub-nanomolar range (IC₅₀s=0.12 nM to 0.25 nM; Log M=−9.9 to −9.6; FIG. 34B, Table 1).

cAMP assays: The signaling properties of the analogs were assessed using intact HEK-293 cells transiently transfected to express with the human PTHR1. Cells were treated with ligand for 30 minutes in the presence of IBMX and the intracellular cAMP levels in the cells were measured by RIA. The analogs were also assayed using HEK-293 cells transiently co-transfected to express with the human PTHR1 and a CRE-Luc cAMP reporter plasmid containing a luciferase reporter gene under transcriptional control of a cAMP-response element-containing promoter. In these assays, the analogs exhibited potencies in the low- to mid-nanomolar range (EC₅₀s˜1 nM to 0.1 nM; Log M=−9.0 to −9.9; (FIGS. 34C and D, Table 1).

### Example 6

### In Vivo Assay of Parathyroid Hormone Analogs

Effects of PTH Analogs on Blood Ca⁺⁺ Levels in Mice.

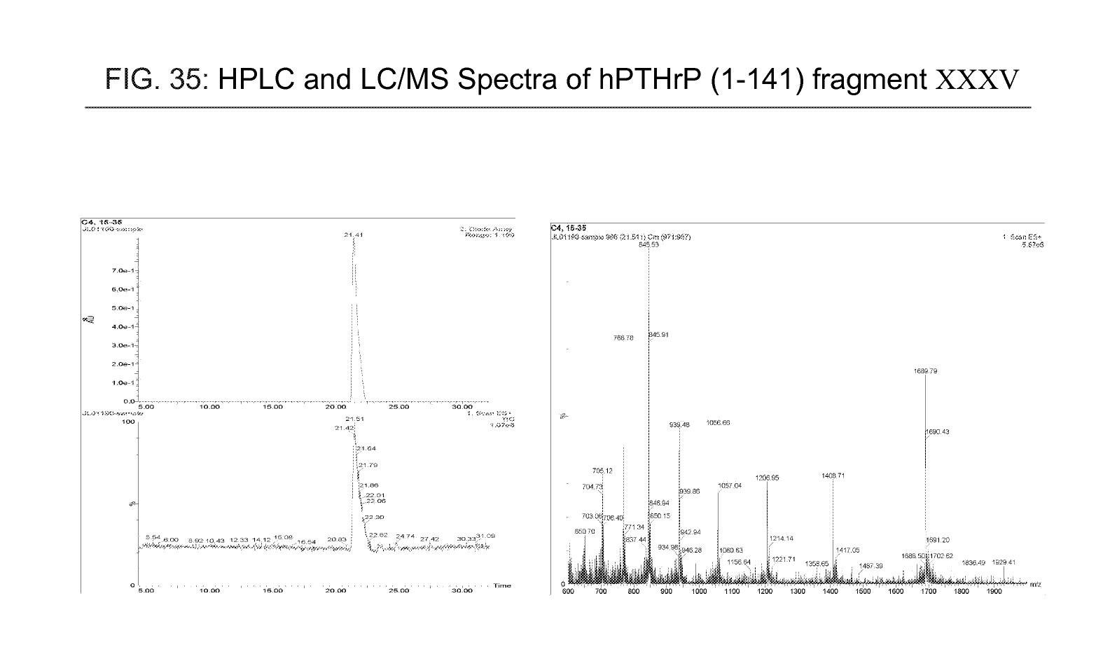

The capacities of the analogs to stimulate increases in blood Ca⁺⁺ were assessed in normal 9 week-old, male, C57BL/6 mice. Prior to injection, the blood Ca⁺⁺ concentrations in the wild-type mice were ˜1.24 mM, FIGS. 35A and 35B). Following injection of the PTH analogs, blood Ca⁺⁺ levels increased robustly and reached a peak of −1.36 mM by one hour post-injection. Blood Ca⁺⁺ levels then returned to vehicle-control levels by six hours with each analog tested.

Materials and Methods

Peptides and Reagents:

PTH derivatives used included humanPTH(1-34)NH₂, and the radioligands ¹²⁵I-PTH(1-34) ([¹²⁵I-[Nle^{8,21}, Tyr³⁴]ratPTH(1-34)NH₂) and ¹²⁵I-M-PTH(1-15) (¹²⁵I-[Aib^{1,3}, Nle⁸, Gln¹⁰, Har¹¹, Ala¹², Trp¹⁴, Tyr¹⁵]PTH(1-15)NH₂), prepared as described.

PTH Binding and Signaling Assays:

Binding to the human PTHR in two pharmacologically distinct conformations, RG and R⁰, was assessed by competition reactions performed in 96-well plates using transiently transfected COS-7 cell membranes. In brief, binding to R⁰, a G protein-independent conformation, was assessed using ¹²⁵I-PTH(1-34) as a tracer radioligand, and including GTPγS (1×10⁻⁵ M) in the reactions. Binding to RG, a G protein-dependent conformation, was assessed using membranes containing a high affinity, negative-dominant Gα_{S} subunit (Gα_{S}^{ND}) and ¹²⁵I-M-PTH(1-15) as a tracer radioligand.

Signaling via the cAMP pathway was assessed in HEK-293 cells transiently transfected to express the human PTHR. The cells in 96-well plates were treated with buffer containing the phosphodiesterase inhibitor, IBMX, and a PTH analog for 30 minutes; the cells were then lysed by replacing the buffer with 50 mM HCl and freezing the plate on dry ice; the cAMP in the lysate was then quantified by MA.

Stimulation of cAMP was also assessed using a CRE-Luc reporter assay using HEK-293 cells transiently co-transfected to express the WT hPTHR along with a cAMP-response-element/luciferase reporter gene construct (Cre-Luc). Cells were treated with ligands in media at 37° C. in a CO₂ incubator for 4-hours, following which the SteadyGlo luciferase reagent (Promega) was added, and luminescence was recorded using a PerkinElmer Envision plate reader.

Measurements of PTH Analog Effects in Mice:

Male mice aged 9 weeks, of strain C57BL/6 were obtained from Charles River laboratory, and treated in accordance with the ethical guidelines adopted by the M.G.H. Mice were injected subcutaneously with vehicle (10 mM citric acid/150 mM NaCl/0.05% Tween-80, pH5.0) or vehicle containing a PTH analog. Peptides were injected at a dose of 20 nmol/kg. Tail vein blood was collected immediately prior to, and at times after injection for analysis of Ca⁺⁺ concentration using a Siemens RapidLab 348 Ca⁺⁺/pH analyzer.

Data Calculations

Data were processed using Microsoft Excel and GraphPad Prism 4.0 software packages.

### Example 7

### Stability Studies of Parathyroid Hormone Analogs

High performance liquid chromatography-mass spectroscopy (HPLC-MS) was used to monitor the degradation of four synthetic compounds over a period of time. Under ambient conditions (room temperature, air, water solution, and neutral pH), the analytical results suggested that natural PTH(1-84) degraded significantly over the time, and after 7 days greater than 90% (estimated based on UV signal) of PTH degraded to fragments or other byproducts. In contrast, analog [Nle^{8,11}]hPTH(1-84) showed much better stability under the same conditions, where less than 10% degradation was observed after 7 days. Two other analogs, hPTH(1-37) and [Nle^{8,11}]hPTH(1-37), showed similar shelf stability, and the analytical results suggested about 70% decomposition after 7 days in both cases.

SEQ ID NO: 1
S₁V₂S₃E₄I₅Q₆L₇M₈H₉N₁₀L₁₁G₁₂K₁₃H₁₄L₁₅N₁₆S₁₇M₁₈E₁₉R₂₀V₂₁E₂₂W₂₃L₂₄R₂₅K₂₆K₂₇L₂₈Q₂₉D₃₀V₃₁
H₃₂N₃₃F₃₄V₃₅A₃₆L₃₇G₃₈A₃₉P₄₀L₄₁A₄₂P₄₃R₄₄D₄₅A₄₆G₄₇S₄₈Q₄₉R₅₀P₅₁R₅₂K₅₃K₅₄E₅₅D₅₆N₅₇V₅₈L₅₉V₆₀
E₆₁S₆₂H₆₃E₆₄K₆₅S₆₆L₆₇G₆₈E₆₉A₇₀D₇₁K₇₂A₇₃D₇₄V₇₅N₇₆V₇₇L₇₈T₇₉K₈₀A₈₁K₈₂S₈₃Q₈₄
SEQ ID NO: 2
X₁V₂S₃E₄I₅Q₆X₇X₈H₉N₁₀L₁₁G₁₂K₁₃H₁₄L₁₅X₁₆S₁₇X₁₈E₁₉R₂₀X₂₁X₂₂W₂₃L₂₄R₂₅X₂₆K₂₇L₂₈Q₂₉D₃₀V₃₁
H₃₂N₃₃F₃₄X₃₅X₃₆L₃₇G₃₈X₃₉X₄₀X₄₁X₄₂X₄₃R₄₄X₄₅X₄₆X₄₇X₄₈Q₄₉R₅₀P₅₁X₅₂K₅₃K₅₄E₅₅X₅₆N₅₇X₅₈X₅₉X₆₀
X₆₁X₆₂X₆₃X₆₄K₆₅S₆₆L₆₇G₆₈E₆₉X₇₀D₇₁K₇₂A₇₃X₇₄V₇₅X₇₆V₇₇L₇₈X₇₉K₈₀X₈₁K₈₂X₈₃Q₈₄
SEQ ID NO: 3
X₈H₉N₁₀L₁₁G₁₂K₁₃H₁₄L₁₅
SEQ ID NO: 4
W₂₃L₂₄R₂₅K₂₆K₂₇L₂₈Q₂₉D₃₀V₃₁H₃₂N₃₃F₃₄
SEQ ID NO: 5
X₈H₉N₁₀L₁₁G₁₂K₁₃H₁₄L₁₅X₁₆S₁₇X₁₈
SEQ ID NO: 6
X₁V₂S₃E₄I₅Q₆X₇M₈H₉N₁₀L₁₁G₁₂K₁₃H₁₄L₁₅X₁₆S₁₇M₁₈E₁₉R₂₀X₂₁X₂₂W₂₃L₂₄R₂₅X₂₆K₂₇L₂₈
Q₂₉D₃₀V₃₁H₃₂N₃₃F₃₄
SEQ ID NO: 7
X₁V₂S₃E₄I₅Q₆X₇X₈H₉N₁₀L₁₁G₁₂K₁₃H₁₄L₁₅X₁₆S₁₇X₁₈E₁₉R₂₀X₂₁X₂₂W₂₃L₂₄R₂₅X₂₆K₂₇L₂₈Q₂₉D₃₀V₃₁
H₃₂N₃₃F₃₄
SEQ ID NO: 8
A₁V₂S₃E₄H₅Q₆L₇L₈H₉D₁₀K₁₁G₁₂K₁₃S₁₄I₁₅Q₁₆D₁₇L₁₈R₁₉R₂₀R₂₁F₂₂F₂₃L₂₄H₂₅H₂₆L₂₇I₂₈A₂₉
E₃₀I₃₁H₃₂T₃₃A₃₄E₃₅I₃₆R₃₇A₃₈T₃₉S₄₀E₄₁V₄₂S₄₃P₄₄N₄₅S₄₆K₄₇P₄₈S₄₉P₅₀N₅₁T₅₂K₅₃N₅₄H₅₅P₅₆
V₅₇R₅₈F₅₉G₆₀S₆₁D₆₂D₆₃E₆₄G₆₅R₆₆Y₆₇L₆₈T₆₉Q₇₀E₇₁T₇₂N₇₃K₇₄V₇₅E₇₆T₇₇Y₇₈K₇₉E₈₀Q₈₁P₈₂L₈₃K₈₄T₈₅
P₈₆G₈₇K₈₈K₈₉K₉₀K₉₁G₉₂K₉₃P₉₄G₉₅K₉₆R₉₇K₉₈E₉₉Q₁₀₀E₁₀₁K₁₀₂K₁₀₃K₁₀₄R₁₀₅R₁₀₆T₁₀₇
R₁₀₈S₁₀₉A₁₁₀W₁₁₁L₁₁₂D₁₁₃S₁₁₄G₁₁₅V₁₁₆T₁₁₇G₁₁₈S₁₁₉G₁₂₀L₁₂₁E₁₂₂G₁₂₃D₁₂₄H₁₂₅L₁₂₆S₁₂₇D₁₂₈
T₁₂₉S₁₃₀T₁₃₁T₁₃₂S₁₃₃L₁₃₄E₁₃₅L₁₃₆D₁₃₇S₁₃₈R₁₃₉R₁₄₀H₁₄₁
SEQ ID NO: 9
A₁V₂S₃E₄H₅Q₆L₇L₈H₉D₁₀K₁₁G₁₂K₁₃S₁₄I₁₅Q₁₆D₁₇L₁₈R₁₉R₂₀R₂₁X₂₂F₂₃L₂₄X₂₅X₂₆L₂₇I₂₈X₂₉
X₃₀X₃₁X₃₂T₃₃A₃₄E₃₅I₃₆R₃₇A₃₈T₃₉S₄₀E₄₁V₄₂S₄₃P₄₄N₄₅X₄₆K₄₇P₄₈X₄₉X₅₀N₅₁T₅₂K₅₃N₅₄X₅₅X₅₆V₅₇R₅₈
F₅₉G₆₀S₆₁X₆₂D₆₃E₆₄G₆₅X₆₆Y₆₇L₆₈T₆₉Q₇₀E₇₁T₇₂N₇₃K₇₄X₇₅X₇₆X₇₇V₇₈K₇₉E₈₀Q₈₁P₈₂
L₈₃K₈₄X₈₅X₈₆G₈₇K₈₈K₈₉K₉₀K₉₁X₉₂K₉₃P₉₄G₉₅K₉₆R₉₇X₉₈E₉₉Q₁₀₀E₁₀₁K₁₀₂K₁₀₃K₁₀₄R₁₀₅R₁₀₆
X₁₀₇R₁₀₈S₁₀₉A₁₁₀W₁₁₁X₁₁₂X₁₁₃S₁₁₄X₁₁₅X₁₁₆X₁₁₇X₁₁₈X₁₁₉X₁₂₀X₁₂₁X₁₂₂X₁₂₃X₁₂₄X₁₂₅X₁₂₆X₁₂₇X₁₂₈
X₁₂₉S₁₃₀X₁₃₁X₁₃₂X₁₃₃X₁₃₄X₁₃₅X₁₃₆X₁₃₇X₁₃₈X₁₃₉X₁₄₀H₁₄₁
SEQ ID NO: 10
H₅Q₆L₇L₈H₉D₁₀K₁₁G₁₂K₁₃S₁₄I₁₅Q₁₆D₁₇L₁₈R₁₉R₂₀R₂₁
SEQ ID NO: 11
T₃₃A₃₄E₃₅I₃₆R₃₇A₃₈T₃₉S₄₀E₄₁V₄₂S₄₃P₄₄N₄₅
SEQ ID NO: 12
V₆₇L₆₈T₆₉Q₇₀E₇₁T₇₂N₇₃K₇₄
SEQ ID NO: 13
E₉₉Q₁₀₀E₁₀₁K₁₀₂K₁₀₃K₁₀₄R₁₀₅R₁₀₆
SEQ ID NO: 14
S₁V₂S₃E₄I₅Q₆L₇M₈H₉N₁₀L₁₁G₁₂K₁₃H₁₄L₁₅N₁₆S₁₇M₁₈E₁₉R₂₀V₂₁E₂₂W₂₃L₂₄R₂₅K₂₆K₂₇L₂₈
Q₂₉D₃₀V₃₁H₃₂N₃₃F₃₄
SEQ ID NO: 15
S₁V₂S₃E₄I₅Q₆L₇M₈H₉N₁₀L₁₁G₁₂K₁₃H₁₄L₁₅N₁₆S₁₇M₁₈E₁₉R₂₀V₂₁E₂₂W₂₃L₂₄R₂₅K₂₆K₂₇L₂₈
Q₂₉D₃₀V₃₁H₃₂N₃₃F₃₄V₃₅A₃₆L₃₇
SEQ ID NO: 16
A₁V₂S₃E₄H₅Q₆L₇L₈H₉D₁₀K₁₁G₁₂K₁₃S₁₄I₁₅Q₁₆D₁₇L₁₈R₁₉R₂₀R₂₁F₂₂F₂₃L₂₄H₂₅H₂₆L₂₇I₂₈A₂₉
E₃₀I₃₁H₃₂T₃₃A₃₄E₃₅I₃₆R₃₇A₃₈T₃₉S₄₀E₄₁V₄₂S₄₃P₄₄N₄₅S₄₆K₄₇P₄₈S₄₉P₅₀N₅₁T₅₂K₅₃N₅₄H₅₅P₅₆
V₅₇R₅₈F₅₉G₆₀S₆₁D₆₂D₆₃E₆₄G₆₅R₆₆Y₆₇L₆₈T₆₉Q₇₀E₇₁T₇₂N₇₃K₇₄V₇₅E₇₆T₇₇V₇₈K₇₉E₈₀Q₈₁P₈₂L₈₃K₈₄T₈₅
P₈₆G₈₇K₈₈K₈₉K₉₀K₉₁G₉₂K₉₃P₉₄G₉₅K₉₆R₉₇K₉₈E₉₉Q₁₀₀E₁₀₁K₁₀₂K₁₀₃K₁₀₄R₁₀₅R₁₀₆T₁₀₇
R₁₀₈S₁₀₉A₁₁₀W₁₁₁L₁₁₂D₁₁₃S₁₁₄G₁₁₅V₁₁₆T₁₁₇G₁₁₈S₁₁₉G₁₂₀L₁₂₁E₁₂₂G₁₂₃D₁₂₄H₁₂₅L₁₂₆S₁₂₇D₁₂₈
T₁₂₉S₁₃₀T₁₃₁T₁₃₂S₁₃₃L₁₃₄E₁₃₅L₁₃₆D₁₃₇S₁₃₈R₁₃₉
SEQ ID NO: 17
A₁V₂S₃E₄H₅Q₆L₇L₈H₉D₁₀K₁₁G₁₂K₁₃S₁₄I₁₅Q₁₆D₁₇L₁₈R₁₉R₂₀R₂₁F₂₂F₂₃L₂₄H₂₅H₂₆L₂₇I₂₈A₂₉
E₃₀I₃₁H₃₂T₃₃A₃₄E₃₅I₃₆R₃₇A₃₈T₃₉S₄₀E₄₁V₄₂S₄₃P₄₄N₄₅S₄₆K₄₇P₄₈S₄₉P₅₀N₅₁T₅₂K₅₃N₅₄H₅₅P₅₆
V₅₇R₅₈F₅₉G₆₀S₆₁D₆₂D₆₃E₆₄G₆₅R₆₆Y₆₇L₆₈T₆₉Q₇₀E₇₁T₇₂N₇₃K₇₄V₇₅E₇₆T₇₇Y₇₈K₇₉E₈₀Q₈₁P₈₂L₈₃K₈₄T₈₅
P₈₆G₈₇K₈₈K₈₉K₉₀K₉₁G₉₂K₉₃P₉₄G₉₅K₉₆R₉₇K₉₈E₉₉Q₁₀₀E₁₀₁K₁₀₂K₁₀₃K₁₀₄R₁₀₅R₁₀₆T₁₀₇
R₁₀₈S₁₀₉A₁₁₀W₁₁₁L₁₁₂D₁₁₃S₁₁₄G₁₁₅V₁₁₆T₁₁₇G₁₁₈S₁₁₉G₁₂₀L₁₂₁E₁₂₂G₁₂₃D₁₂₄H₁₂₅L₁₂₆S₁₂₇D₁₂₈
T₁₂₉S₁₃₀T₁₃₁T₁₃₂S₁₃₃L₁₃₄E₁₃₅L₁₃₆D₁₃₇S₁₃₈R₁₃₉T₁₄₀A₁₄₁L₁₄₂L₁₄₃W₁₄₄G₁₄₅L₁₄₆K₁₄₇K₁₄₈K₁₄₉
K₁₅₀E₁₅₁N₁₅₂N₁₅₃R₁₅₄R₁₅₅T₁₅₆H₁₅₇H₁₅₈M₁₅₉Q₁₆₀L₁₆₁M₁₆₂I₁₆₃S₁₆₄L₁₆₅F₁₆₆K₁₆₇S₁₆₈P₁₆₉L₁₇₀
L₁₇₁L₁₇₂L₁₇₃
SEQ ID NO: 18
S₁V₂S₃E₄I₅Q₆L₇M₈H₉N₁₀L₁₁G₁₂K₁₃H₁₄L₁₅N₁₆S₁₇M₁₈E₁₉R₂₀V₂₁E₂₂W₂₃L₂₄R₂₅K₂₆K₂₇L₂₈Q₂₉D₃₀V₃₁
H₃₂N₃₃F₃₄V₃₅A₃₆L₃₇G₃₈A₃₉

We claim:

1. A parathyroid hormone peptide 1-84 amino acids in length having an amino acid sequence ≧80% identical to SEQ ID NO: 2, wherein the parathyroid hormone peptide includes a non-natural amino acid at one or more positions corresponding to residues X₁, X₇, X₈, X₁₆, X₁₈, X₂₁, X₂₂, X₂₆, X₃₅, X₃₆, X₃₉, X₄₀, X₄₁, X₄₂, X₄₃, X₄₅, X₄₆, X₄₇, X₄₈, X₅₂, X₅₆, X₅₈, X₅₉, X₆₀, X₆₁, X₆₂, X₆₃, X₆₄, X₇₀, X₇₄, X₇₆, X₇₉, X₈₁ or X₈₃.

2. The parathyroid hormone peptide of claim 1, wherein the parathyroid hormone includes at least one norleucine (Nle) and/or methoxinine (Mox) residue.

3. The parathyroid hormone peptide of claim 1, wherein the parathyroid hormone peptide includes a norleucine and/or methoxinine residue at a position corresponding to residue 8, residue 18 and combinations thereof.

4. The parathyroid hormone peptide of claim 1, wherein the peptide includes at least one of SEQ ID NO: 3, SEQ ID NO: 4 and SEQ ID NO: 5.

5. The parathyroid hormone peptide of claim 3, wherein at least one of the following is true:
X₁ is S, A, Nle or Mox;
X₇ is F, L, Nle or Mox;
X₁₆ is N, S, A, Nle or Mox;
X₁₈ is M, L, V, Nle or Mox;
X₂₁ is V, M, Nle or Mox; and
X₂₂ is E, Q, Nle or Mox.

6. The parathyroid hormone peptide of claim 4, wherein at least one of the following is true:
X₃₆ is A, Nle or Mox;
X₃₉ is A, Nle or MOX;
X₄₅ is D, Nle or Mox;
X₄₈ is S, Nle or Mox;
X₅₆ is D, Nle or Mox;
X₅₈ is V, Nle or Mox;
X₆₀ is V, Nle or Mox;
X₆₁ is E, Nle or Mox;
X₆₂ is E, Nle or Mox;
X₇₀ is A, Nle or Mox;
X₇₄ is D, Nle or Mox; and
X₈₁ is A, Nle or Mox.

7. The parathyroid hormone peptide of claim 1, wherein the peptide is glycosylated with at least one glycan group.

8. The parathyroid hormone peptide of claim 7, wherein the peptide is glycosylated at a serine or threonine residue.

9. The peptide of claim 8, wherein the at least one glycan group is selected from

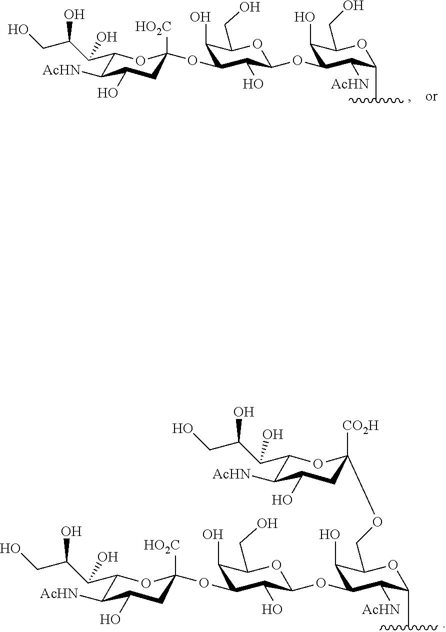

10. The peptide of claim 8, wherein the at least one glycan group is

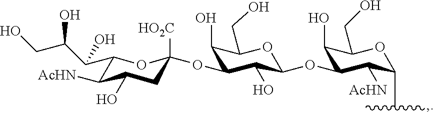

11. The peptide of claim 8, wherein the at least one glycan group is

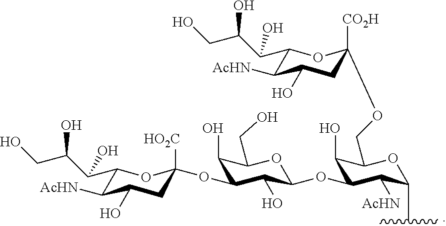

12. The peptide of claim 7, wherein the peptide is glycosylated at a asparagine or glutamine residue.

13. The peptide of claim 12, wherein the at least one glycan group is selected from:

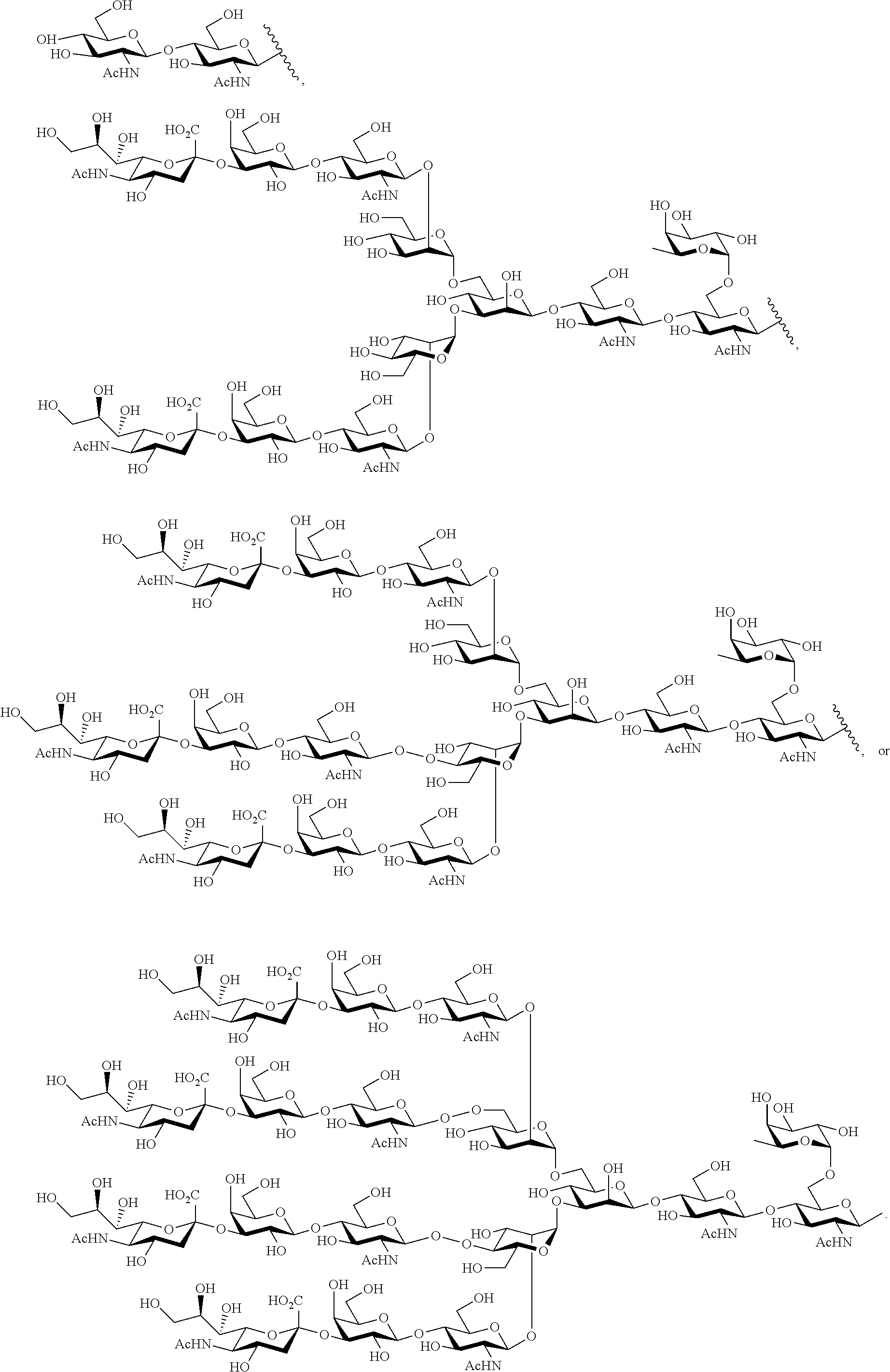

14. The peptide of claim 13, wherein the at least one glycan group is

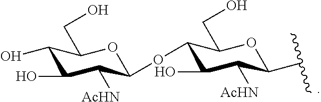

15. The peptide of claim 13, wherein the at least one glycan group is

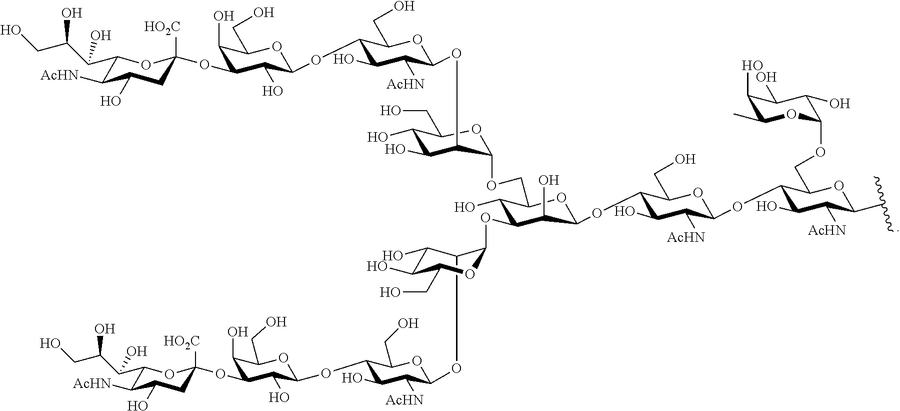

16. The peptide of claim 13, wherein the at least one glycan group is

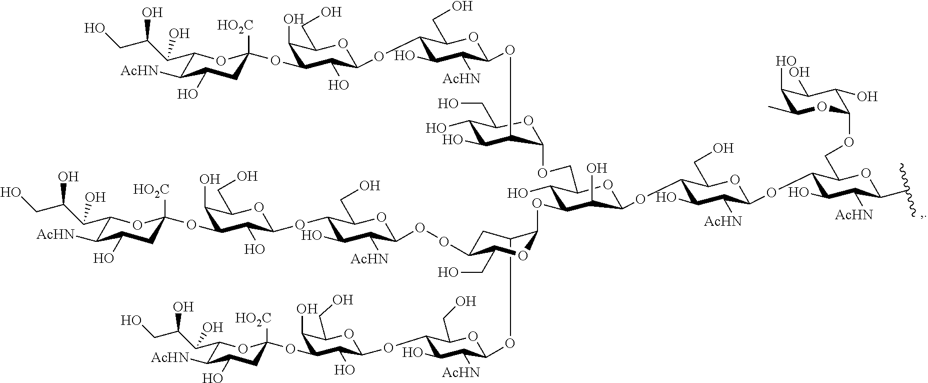

17. The peptide of claim 13, wherein the at least one glycan group is

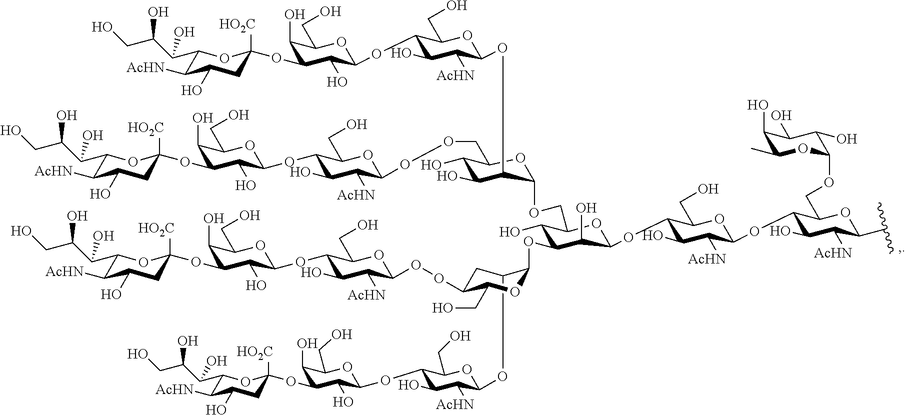

18. A parathyroid hormone peptide 1-37 amino acids in length having an amino acid sequence ≧80% identical to SEQ ID NO: 15, wherein the parathyroid hormone peptide includes a norleucine and/or methoxinine residue at a position corresponding to residue 8, residue 18 and combinations thereof.

19. A parathyroid hormone peptide having an amino acid sequence which includes an element ≧80% identical to SEQ ID NO: 14, wherein the parathyroid hormone peptide includes a norleucine and/or methoxinine residue at a position corresponding to residue 8, residue 18 and combinations thereof.

20. A parathyroid hormone-related peptide 1-141 amino acids in length having an amino acid sequence ≧80% identical to SEQ ID NO: 8.

21. A pharmaceutical composition comprising the parathyroid hormone peptide of claim 2, the glycosylated parathyroid hormone fragment of claim 7, or the parathyroid hormone-related protein of claim 18 and a pharmaceutically acceptable excipient.

22. A method of preparing a biologically active hormone or glycopeptide comprising at least one native chemical ligation coupling at an amino acid residue other than cysteine or methionine.

23. The method of claim 22, wherein the native chemical ligation coupling occurs at a residue selected from alanine, valine, threonine, leucine and proline.

24. The method of claim 22, wherein the biologically active hormone is selected from parathyroid hormone (1-34), parathyroid hormone (1-37), parathyroid hormone (1-39), parathyroid hormone (1-84), N-glycosylated parathyroid hormone, O-glycosylated parathyroid hormone, parathyroid hormone-related protein (1-139), parathyroid hormone-related protein (1-141), parathyroid hormone-related protein (1-173).

25. The method of claim 24, wherein the biologically active hormone is parathyroid hormone (1-34).

26. The method of claim 25 comprising the steps of:
(i) preparing fragment V via the native chemical ligation of fragments I and II: 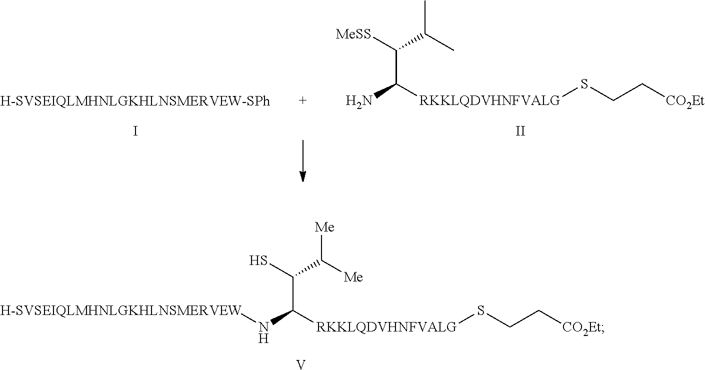
(ii) preparing fragment VI via the native chemical ligation of fragments III and IV: 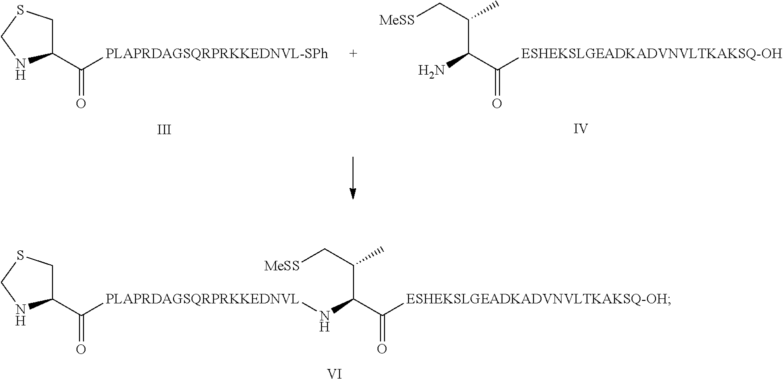
(iii) deprotecting fragment VI to produce fragment VII: 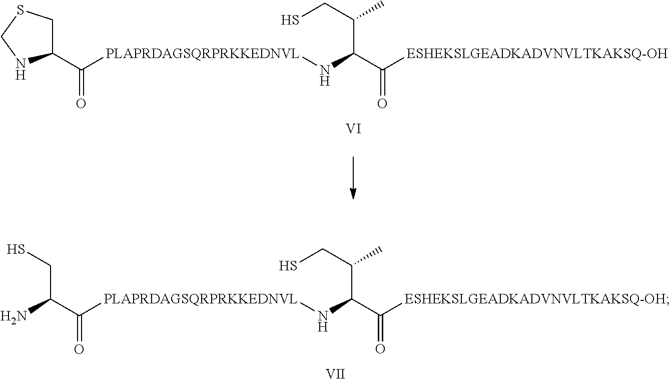
(iv) coupling of fragments V and VII via native chemical ligation to produce fragment VIII: 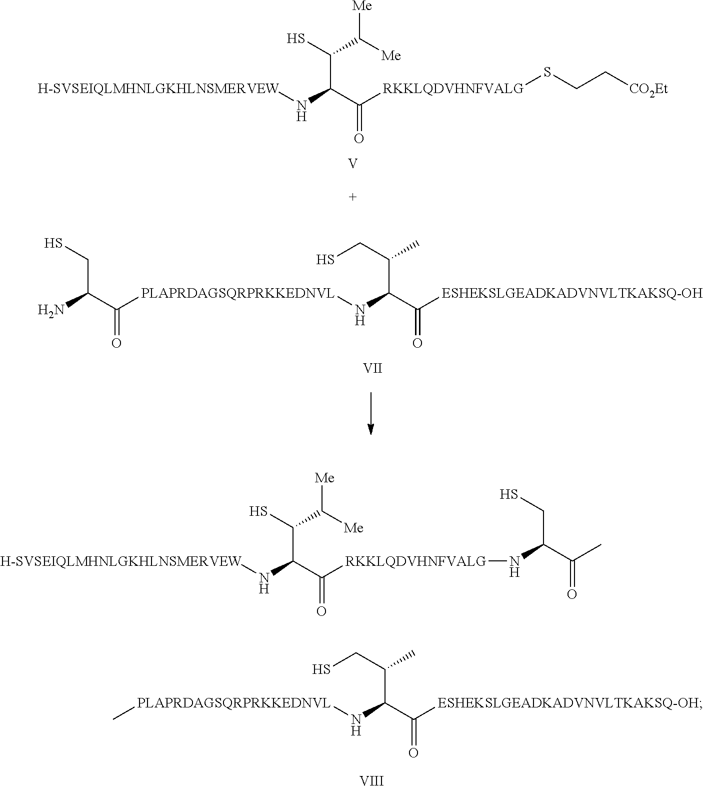
and (v) reducing fragment VIII to produce an hPTH peptide:

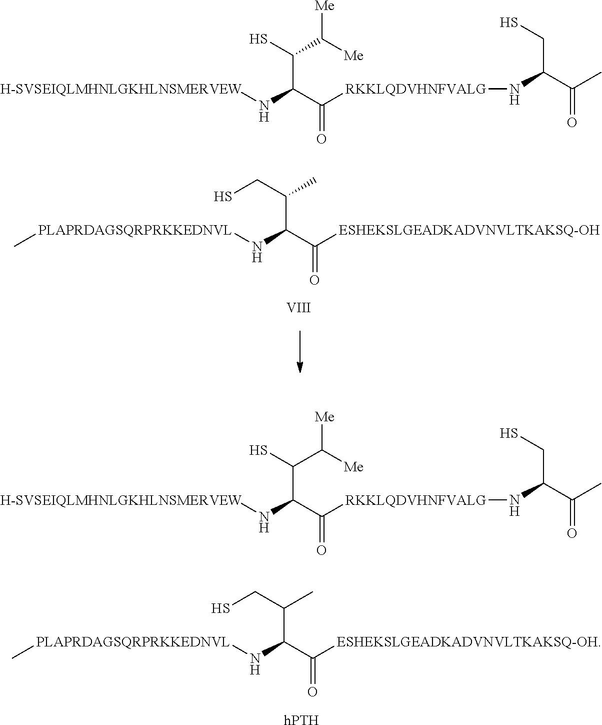

27. The method of claim 24, wherein the biologically active hormone is parathyroid hormone-related protein (1-141).

28. The method of claim 27 comprising the native chemical ligation coupling of fragments XXX, XXXI, XXXII and XXXIII.

29. A native chemical ligation fragment selected from the group consisting of: I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI or XXXVII.

30. A method of treating a disease, disorder and/or symptom associated with hypoparathyroidism comprising administering a therapeutically effective amount of a hPTH or hPTHrP peptide and/or analog.

31. The method of claim 30, wherein the disease, disorder and/or symptom is selected from osteoporosis, hypocalcemia and hypocalciuria.


SEQUENCE LISTING (18 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 2 - protein, 84 aa, Artificial Sequence
  synthetic
XVSEIQXXHNLGKHLXSXERXXWLRXKLQDVHNFXXLGXXXXXRXXXXQRPXKKEXNXXXXXXXKSLGEXDKAXVXVLXKXKXQ

SEQ ID NO: 3 - protein, 8 aa, Artificial Sequence
  synthetic
XHNLGKHL

SEQ ID NO: 4 - protein, 12 aa, Artificial Sequence
  synthetic
WLRKKLQDVHNF

SEQ ID NO: 5 - protein, 11 aa, Artificial Sequence
  synthetic
XHNLGKHLXSX

SEQ ID NO: 6 - protein, 34 aa, Artificial Sequence
  synthetic
XVSEIQXMHNLGKHLXSMERXXWLRXKLQDVHNF

SEQ ID NO: 7 - protein, 34 aa, Artificial Sequence
  synthetic
XVSEIQXXHNLGKHLXSXERXXWLRXKLQDVHNF

SEQ ID NO: 8 - protein, 141 aa, Homo sapiens
AVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 9 - protein, 141 aa, Artificial Sequence
  synthetic
AVSEHQLLHDKGKSIQDLRRRXFLXXLIXXXXTAEIRATSEVSPNXKPXXNTKNXXVRFGSXDEGXYLTQETNKXXXYKEQPLKXXGKKKKXKPGKRXEQEKKKRRXRSAWXXSXXXXXXXXXXXXXXXSXXXXXXXXXXH

SEQ ID NO: 10 - protein, 17 aa, Artificial Sequence
  synthetic
HQLLHDKGKSIQDLRRR

SEQ ID NO: 11 - protein, 13 aa, Artificial Sequence
  synthetic
TAEIRATSEVSPN

SEQ ID NO: 12 - protein, 8 aa, Artificial Sequence
  synthetic
YLTQETNK

SEQ ID NO: 13 - protein, 8 aa, Artificial Sequence
  synthetic
EQEKKKRR

SEQ ID NO: 14 - protein, 34 aa, Artificial Sequence
  synthetic
SVSEIQLMHNLGKHLNSMERVEWLRKKLQDVHNF

SEQ ID NO: 15 - protein, 37 aa, Artificial Sequence
  synthetic
SVSEIQLMHNLGKHLNSMERVEWLRKKLQDVHNFVAL

SEQ ID NO: 16 - protein, 139 aa, Homo sapiens
AVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSR

SEQ ID NO: 17 - protein, 173 aa, Homo sapiens
AVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRTALLWGLKKKKENNRRTHHMQLMISLFKSPLLLL

SEQ ID NO: 18 - protein, 39 aa, Artificial Sequence
  synthetic
SVSEIQLMHNLGKHLNSMERVEWLRKKLQDVHNFVALGA